(19) United States
(12) Patent Application Publication
(10) Pub. No.: US 2008/0234639 A1
(43) Pub. Date: Sep. 25, 2008

(54) Valves, Valved Fluid Transfer Devices and Ambulatory Infusion Devices Including The Same

(76) Inventors: David Christopher Antonio, Pasadena, CA (US); Stephen Roy Hessel, Valencia, CA (US); Scott R. Gibson, Granada Hills, CA (US); Christian Michael Merot, Santa Paula, CA (US); Lawrence Scott Ring, Valencia, CA (US); Norbert William Frenz, Williamsville, NY (US); Stephen D. Das, The Woodlands, TX (US)

Correspondence Address: HENRICKS SLAVIN AND HOLMES, LLP;ADVANCED BIONICS CORPORATION, 840 APOLLO STREET, SUITE 200, EL SEGUNDO, CA, 90245, US

(21) Appl. No.: 12/054,261

(22) Filed: Mar. 24, 2008

Related U.S. Application Data
(60) Provisional application No. 60/896,910, filed on Mar. 24, 2007.
(60) Provisional application No. 60/896,911, filed on Mar. 24, 2007.

(51) Int. Cl. A61M 5/00

(52) U.S. Cl. 604/249

(57) ABSTRACT

Valves, valved fluid transfer devices and ambulatory infusion devices including the same.

### CROSS-REFERENCE TO RELATED APPLICATIONS

This application claims the benefit of U.S. Provisional Application Ser. No. 60/896,910, filed Mar. 24, 2007 and entitled “Valves, Valved Fluid Transfer Devices and Ambulatory Infusion Devices Including The Same” and U.S. Provisional Application Ser. No. 60/896,911, filed Mar. 24, 2007 and entitled “Valves, Valved Fluid Transfer Devices and Ambulatory Infusion Devices Including The Same,” both of which is incorporated herein by reference in their entirety.

### BACKGROUND OF THE INVENTIONS

1. Field of Inventions

The present inventions relate generally to valves, valved fluid transfer devices and ambulatory infusion devices including the same.

2. Description of the Related Art

Ambulatory infusion devices, such as implantable infusion devices and externally carried infusion devices, have been used to provide a patient with a medication or other substance (collectively “infusible substance”) and frequently include a reservoir and a fluid transfer device. The reservoir is used to store the infusible substance and is coupled to the fluid transfer device which is, in turn, connected to an outlet port. A catheter, which has one or more outlets at the target body region, may be connected to the outlet port. As such, infusible the reservoir may be transferred from the reservoir to the target body region by way of the fluid transfer device and catheter.

The fluid transfer devices in ambulatory infusion devices frequently include a pump, such as an electromagnet pump, and one or more valves. The present inventors have determined that the valves employed in such fluid transfer devices are susceptible to improvement. For example, the present inventors have determined that the main check valves are susceptible to improvement.

### BRIEF DESCRIPTION OF THE DRAWINGS

Detailed descriptions of exemplary embodiments will be made with reference to the accompanying drawings.

FIG. 1 is a side, partial section view of a fluid transfer device in accordance with various embodiments of some of the present invention.

FIGS. 2-5 are section views showing the fluid transfer device illustrated in FIG. 1 in various states.

FIG. 6 is a partial section view of a valve in accordance with one embodiment of a present invention.

FIG. 7 is a section view of a portion of the valve illustrated in FIG. 6.

FIG. 8 is a section view of a portion of the valve illustrated in FIG. 6 in an open state.

FIG. 9 is a section view of a portion of the valve illustrated in FIG. 6 in a closed state.

FIG. 10 is a partial section view of a portion of a fluid transfer device including the valve illustrated in FIG. 6.

FIG. 11 is a partial section view of a valve in accordance with one embodiment of a present invention.

FIG. 12 is a section view of a portion of the valve illustrated in FIG. 11 in an open state.

FIG. 13 is a section view of a portion of the valve illustrated in FIG. 11 in a closed state.

FIG. 14 is a partial section view of a portion of a fluid transfer device including the valve illustrated in FIG. 11.

FIG. 15 is a partial section view of a valve in accordance with one embodiment of a present invention.

FIG. 15A is a section view of a portion of a valve element in accordance with one embodiment of a present invention.

FIG. 16 is a partial section view of a valve in accordance with one embodiment of a present invention.

FIG. 17 is a partial section view of a portion of a fluid transfer device including the valve illustrated in FIG. 16.

FIG. 18 is a partial section view of a valve in accordance with one embodiment of a present invention.

FIG. 19 is a partial section view of a portion of a fluid transfer device including the valve illustrated in FIG. 18.

FIG. 20 is a partial section view of a valve in accordance with one embodiment of a present invention.

FIG. 21 is a partial section view of a portion of a fluid transfer device including the valve illustrated in FIG. 20.

FIG. 22 is a partial section view of a valve in accordance with one embodiment of a present invention.

FIG. 23 is a section view of the valve seat illustrated in FIG. 22.

FIG. 24 is a partial section view of a portion of a fluid transfer device including the valve illustrated in FIG. 22.

FIG. 25 is a partial section view of a valve in accordance with one embodiment of a present invention.

FIG. 26 is an enlarged view of a portion of FIG. 25.

FIG. 27 is a partial section view of a portion of a fluid transfer device including the valve illustrated in FIG. 25.

FIG. 28 is a partial section view of a valve in accordance with one embodiment of a present invention.

FIG. 29 is a partial section view of a portion of a fluid transfer device including the valve illustrated in FIG. 28.

FIG. 30 is a partial section view of a valve in accordance with one embodiment of a present invention.

FIG. 31 is a plan view of the valve seat illustrated in FIG. 30.

FIG. 32 is a plan view of another valve seat that may be used in the valve illustrated in FIG. 30.

FIG. 33 is a partial section view of a portion of a fluid transfer device including the valve illustrated in FIG. 30.

FIG. 34 is a partial section view of a valve in accordance with one embodiment of a present invention.

FIG. 35 is a partial section view of a portion of a fluid transfer device including the valve illustrated in FIG. 34.

FIG. 36 is a partial section view of a valve in accordance with one embodiment of a present invention.

FIG. 37 is an enlarged view of a portion of FIG. 36.

FIG. 38 is a partial section view of a portion of a fluid transfer device including the valve illustrated in FIG. 36.

FIG. 39 is a partial section view of a valve in accordance with one embodiment of a present invention.

FIG. 40 is a partial section view of a portion of a fluid transfer device including the valve illustrated in FIG. 39.

FIG. 41 is a partial section view of a valve in accordance with one embodiment of a present invention.

FIG. 42 is an enlarged view of a portion of the valve element illustrated in FIG. 41.

FIG. 43 is a partial section view of a portion of a fluid transfer device including the valve illustrated in FIG. 41.

FIG. 44 is a partial section view of a valve in accordance with one embodiment of a present invention.

FIG. 45 is an enlarged view of a portion of the valve illustrated in FIG. 44.

FIG. 46 is a partial section view of a portion of a fluid transfer device including the valve illustrated in FIG. 44.

FIG. 47 is a partial section view of a portion of a fluid transfer device illustrated in FIGS. 1-5

FIG. 48 is a partial section view of a portion of the fluid transfer device illustrated in FIG. 44.

FIG. 49 is a partial section view of a valve in accordance with one embodiment of a present invention.

FIG. 49A is an enlarged view of a portion of the gasket illustrated in FIG. 49.

FIG. 49B is a plan view of the valve seat illustrated in FIG. 49.

FIG. 49C is a section view taken along line 49C-49C in FIG. 49B.

FIG. 50 is an enlarged view of a portion of the valve element illustrated in FIG. 49.

FIG. 51 is a partial section view of a portion of a fluid transfer device including the valve illustrated in FIG. 49.

FIG. 52 is a section view showing the valve illustrated in FIG. 49 in an assembly fixture in accordance with one embodiment of a present invention.

FIG. 53 is an enlarged view of a portion of FIG. 52.

FIG. 54 is a section view of a valve in accordance with one embodiment of a present invention.

FIG. 55 is an enlarged view of a portion of the valve illustrated in FIG. 54.

FIG. 56 is a plan view of the spring illustrated in FIG. 54.

FIG. 57 is a partial section view of a portion of a fluid transfer device including the valve illustrated in FIG. 54.

FIG. 58 is a partial section view of a portion of the fluid transfer device illustrated in FIG. 54.

FIG. 59 is a section view of a valve in accordance with one embodiment of a present invention.

FIG. 60 is an enlarged view of a portion of FIG. 59.

FIG. 61 is a section view taken along line 61-61 in FIG. 59.

FIG. 62 is a partial section view of a portion of a fluid transfer device including the valve illustrated in FIG. 59.

FIG. 63 is a partial section view of a valve in accordance with one embodiment of a present invention.

FIG. 64 is a top view of a portion of the valve illustrated in FIG. 63.

FIG. 64A is an enlarged view of a portion of the gasket illustrated in FIG. 63.

FIG. 65 is an enlarged view of a portion of the valve element illustrated in FIG. 63.

FIG. 66 is a partial section view of a portion of a fluid transfer device including the valve illustrated in FIG. 63.

FIG. 67 is a block diagram of a fluid transfer device.

FIG. 68 is a plan view of an implantable infusion device in accordance with one embodiment of a present invention.

FIG. 69 is a plan view of the implantable infusion device illustrated in FIG. 68 with the cover removed.

FIG. 70 is a partial section view taken along line 70-70 in FIG. 68.

FIG. 71 is a block diagram of the implantable infusion device illustrated in FIGS. 68-70.

FIG. 72 is a plan view of an implantable infusion device in accordance with one embodiment of a present invention.

FIG. 73 is a schematic view of the implantable infusion device illustrated in FIG. 72.

FIG. 74 is a partial section view of a valve in accordance with one embodiment of a present invention.

FIG. 75 is a partial section view of a portion of a valve in accordance with one embodiment of a present invention.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. The present inventions have application in a wide variety of apparatus. One example is an electromagnet pump-based fluid transfer device that may be employed in an implantable infusion device, and the present inventions are discussed in the context of electromagnet pump-based fluid transfer devices and implantable infusion devices. The present inventions are not, however, limited to electromagnet pump-based fluid transfer devices and implantable infusion devices and are instead also applicable to other fluid transfer devices and infusion devices that currently exist, or are yet to be developed. For example, the present inventions are applicable to fluid transfer devices with solenoid pumps, piezoelectric pumps, and any other mechanical or electromechanical pulsatile pump as well as externally carried infusion devices.

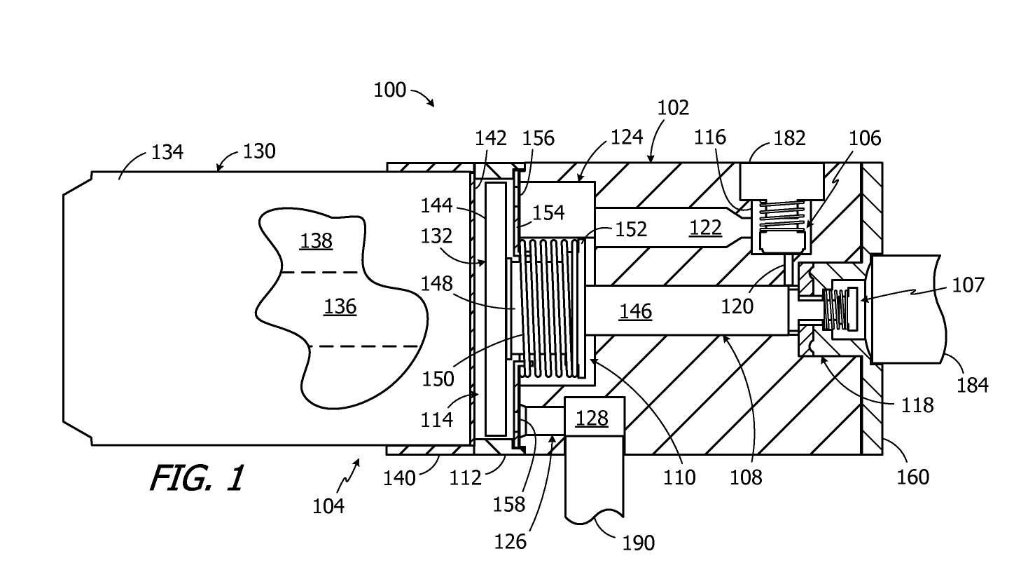

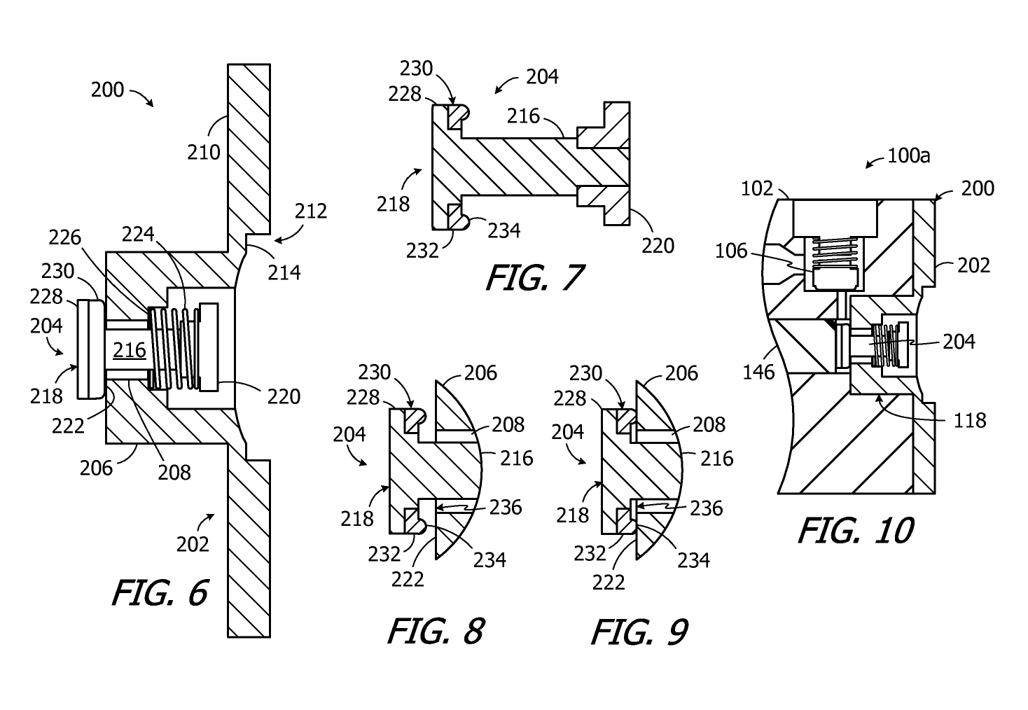

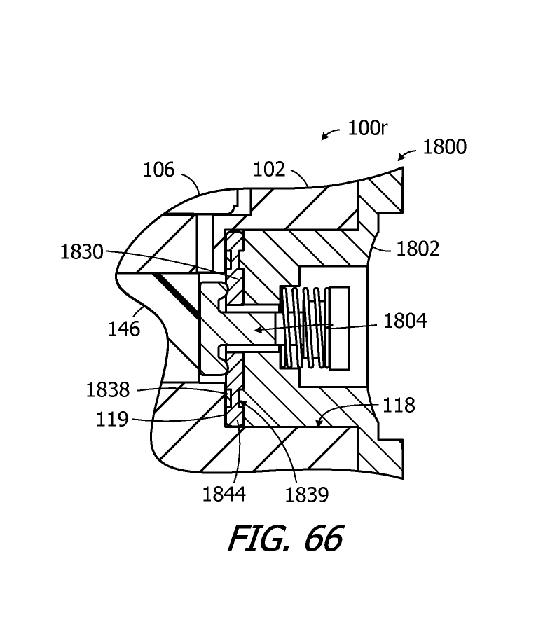

One example of a fluid transfer device is illustrated in FIGS. 1-5. The exemplary fluid transfer device, which is generally represented by reference numeral 100, includes a housing 102, an electromagnet pump 104, a bypass valve 106 and a main check valve 107. The exemplary fluid transfer device may also include any of the main check valves 200-1800 described below with reference to FIGS. 6-66 in place of the main check valve 107. The main check valve 107 illustrated in FIGS. 1-5 is a conventional valve that is merely being used for the purpose of explaining the overall operation of the fluid transfer device 100 and explaining some of the shortcomings associated with conventional valves.

The housing 102 in the exemplary fluid transfer device 100 is a generally solid, cylindrical structure with various open regions. The open regions accommodate portions of structures, such as the electromagnet pump 104, bypass valve 106, main check valve 107, and also define a fluid flow path. More specifically, the housing 102 includes a piston bore 108 and a hub recess 110 that respectively receive the electromagnet pump armature piston 146 and armature hub 148 (discussed below). A weld ring 112, which is secured to the end of the housing 102 opposite the main check valve 107, defines a pole recess 114 for the armature pole 144 (discussed below). A pair of valve recesses 116 and 118 for the bypass valve 106 and main check valve 107 are also provided. With respect to the fluid flow path, the housing 102 includes an orifice 120 that extends from the piston bore 108 to the bypass valve recess 116, a bypass fluid chamber 122, fluid passages 124 and 126, and an outlet recess 128. Additionally, and although it is not limited to any particular material(s), the exemplary housing 102 is formed from titanium.

Turning to the pump portion of the exemplary fluid transfer device 100, the electromagnet pump 104 includes an electromagnet 130 and an armature 132. The electromagnet 130, which is carried within a case 134, includes a core 136 and a coil 138. The case 134 and core 136 are made from a magnetic material. The coil 138 consists of a wire or other conductor that is wound around the core 136. The coil 138 may be insulated from the case 134 by electrically non-conductive spacers (not shown), which center the coil within the case, or through the use of potting compound or encapsulant material between the case and the coil.

The electromagnet case 134 is secured to the housing 102 in the exemplary fluid transfer device 100 through the use of the aforementioned weld ring 112 on the housing and a weld ring 140 on the case. More specifically, the outer diameters of the weld rings 112 and 140 are substantially equal to one another and the outer surfaces thereof are substantially flush. During assembly, the housing 102 and the electromagnet case 134 are positioned on opposite sides of a barrier 142 and are then secured to one another by a weld (not shown) joining the outer surfaces of the weld rings 112 and 140. The barrier separates the pole recess 114, which will ultimately be filled with fluid, from the electromagnet 130.

The armature 132 in the illustrated embodiment is positioned within a fluid containing region of the housing that is defined by the piston bore 108, the hub recess 110 and the pole recess 114. The exemplary armature 132 consists of a pole 144 formed from a magnetic material (e.g. magnetic steel), which is located within the pole recess 114 such that it will be magnetically attracted to the electromagnet 130 when the electromagnet is actuated, and a cylindrically-shaped piston 146 that extends from the pole and through the piston bore 108 to the main check valve 107. A hub 148 is located within the hub recess 110 and is used to secure the pole 144 to the piston 146. A main spring 150 biases the armature 132 to the “rest” position illustrated in FIG. 1. The main spring 150 is compressed between a spring retainer 152 on the hub 148 and a spring retainer plate 154. The spring retainer plate 154, which is held in place by the housing 102 and the weld ring 112, includes an inlet opening 156 that allows fluid to pass from the fluid passage 124 to the pole recess 114 and an outlet opening 158 that allows fluid to pass from the pole recess to the fluid passage 126.

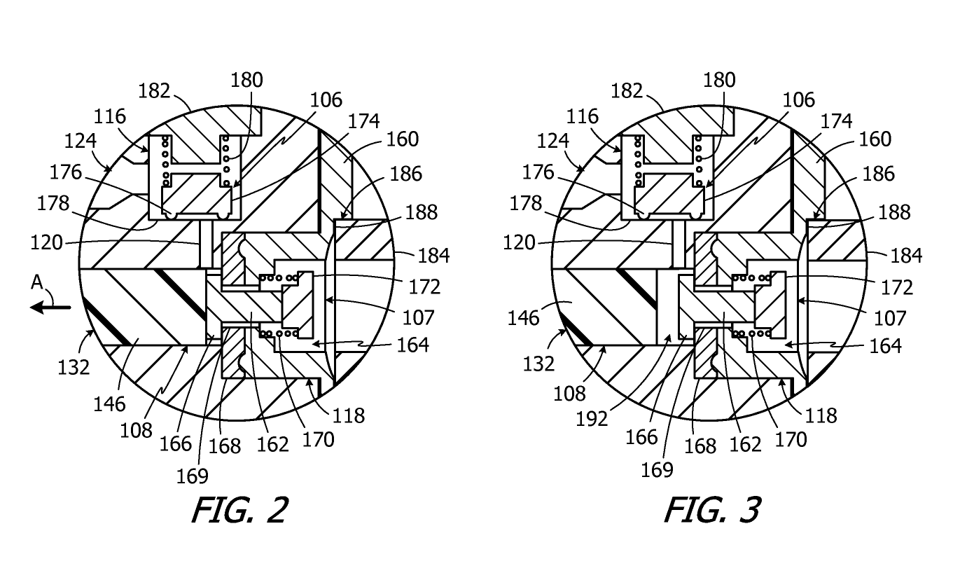

Turning to FIG. 2, the main check valve 107 includes a housing 160, which may be positioned within the valve recess 118 and secured to the housing 102, and a valve element (or “plunger”) 162 that is movable relative to the housing 160 within a fluid lumen 164. The valve element 162 includes a head 166 that abuts an elastomeric valve seat 168 when the main check valve 107 is in the closed state illustrated in FIG. 2. The shaft portion of the valve element 162 passes through an opening 169 in the valve seat 168. The valve element 162 is biased to the closed position by a spring 170. One end of the spring 170 abuts the housing 160 and the other end abuts a spring retainer 172 that is secured to the valve element 162.

The exemplary bypass valve 106 includes a valve element 174 with an integral sealing ring 176. The sealing ring 176, which has a semi-circular cross-sectional shape, engages the wall 178 that defines the end of the valve recess 116 and surrounds the orifice 120 when in the closed position illustrated in FIG. 2. Otherwise identical valve elements without the sealing ring may also be employed in the bypass valve. Suitable materials for the valve element 174 include elastomers such as, for example, silicone rubber, latex rubber, urethane, butyl rubber, and isoprene. In other implementations, the valve element 174 may be formed in whole or in part from a metal. The valve element 174 is biased to the closed position by a spring 180. One end of the spring 180 abuts the valve element 174, while the other end abuts a plug 182 that may be secured to housing 102 to maintain the bypass valve 106 within the valve recess 116. The plug 182 also forms a fluid tight seal which prevents fluid from escaping from the housing 102 by way of the valve recess 116.

Fluid may be supplied to the exemplary fluid transfer device 100 illustrated in FIG. 1 by way of an inlet tube 184. To that end, and referring to FIG. 2, the main check valve housing 160 includes a recess 186, with a shoulder 188, that receives the inlet tube 184. A filter (not shown) may be positioned within the recess 186 between the inlet tube 184 and the shoulder 188. Fluid exits the fluid transfer device 100 by way of an outlet tube 190 that is received within the outlet recess 128 in the housing 102.

The exemplary fluid transfer device 100 operates as follows. Referring first to FIGS. 1 and 2, the fluid transfer device 100 is shown here in the “rest” state. The armature 132 is in the rest position, the electromagnet 130 is not energized, and the bypass valve 106 and main check valve 107 are both closed. Under normal operating conditions, there will be no flow through the fluid transfer device 100 when the fluid transfer device is in the rest state and the valves 106 and 107 are closed. Although sufficient pressure at the inlet tube 184 could result in the flow through the fluid transfer device 100 while the fluid transfer device is in the rest state illustrated in FIG. 2, the likelihood that this could occur is greatly reduced by maintaining the fluid source at a relatively low pressure.

The exemplary fluid transfer device 100 is actuated by connecting the coil 138 in the electromagnet 130 to an energy source (e.g. one or more capacitors that are being fired). The resulting magnetic field is directed through the core 136 and into, as well as through, the armature pole 144. The armature pole 144 is attracted to the core 136 by the magnetic field. The intensity of the magnetic field grows as current continues to flow through the coil 138. When the intensity reaches a level sufficient to overcome the biasing force of the main spring 150, the armature 132 will be pulled rapidly in the direction of arrow A (FIG. 2) until the armature pole 144 reaches the barrier 142. The armature piston 146 and hub 148 will move with armature pole 144 and compress the spring main 140. This is also the time at which fluid exits the fluid transfer device 100 by way of the passage 126 and the outlet tube 190.

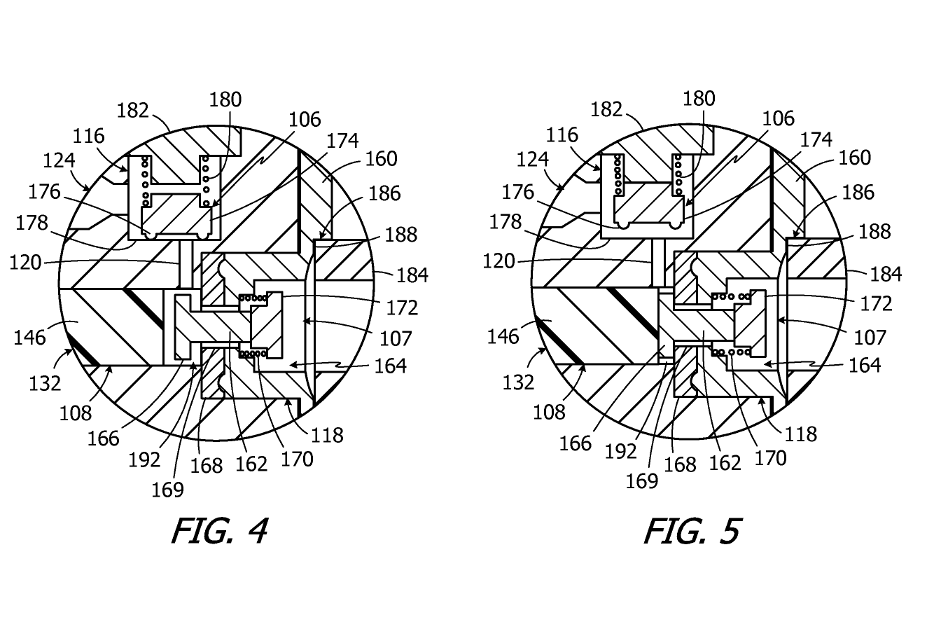

Movement of the armature piston 146 from the position illustrated in FIG. 2 to the position illustrated in FIG. 3 results in a decrease in pressure in the pump chamber 192, i.e. the volume within the piston bore 108 between the armature piston 146 and the valve seat 168. The coil will continue to be energized for a brief time (e.g. a few milliseconds) in order to hold the armature piston 146 in the location illustrated in FIG. 3. The reduction in pressure within the pump chamber 192 will open the main check valve 107 by overcoming the biasing force of the spring 170 and move valve element 162 to the position illustrated in FIG. 4. As a result, the valve head 166 will move away from the valve seat 168 and fluid will flow into the pump chamber 192. The main check valve 107 will close, due to the force exerted by spring 170 on valve element 162, once the pressure within pump chamber 192 is equal to pressure at the inlet tube 184. However, because the coil 138 continues to be energized, the armature 132 will remain in the position illustrated in FIGS. 3 and 4 as fluid flows into the pump chamber 192 and the main check valve 107 closes.

Immediately after the main check valve 107 closes, the coil 138 will be disconnected from the energy source and the magnetic field established by the electromagnet 130 will decay until it can no longer overcome the force exerted on the armature 132 by the main spring 150. The armature 132 will then move back to the position illustrated in FIGS. 2 and 5. The associated increase in pressure within the pump chamber 192 is sufficient to open the bypass valve 106 by overcoming the biasing force of the spring 180 and moving the valve element 174 to the position illustrated in FIG. 5. The increase in pressure within the pump chamber 192, coupled with movement of the valve element away from the wall 178, results in the fluid flowing through the orifice 120 to the fluid chamber 122. The flow of fluid will cause the pressure in the orifice 120 and the fluid chamber 122 to equalize. At this point, the bypass valve 106 will close, due to the force exerted by spring 180 on the valve element 174, thereby returning the exemplary fluid transfer device 110 to the rest state illustrated in FIG. 2.

Additional information concerning the exemplary fluid transfer device 100, as well as other fluid transfer devices, may be found in U.S. Pat. Nos. 6,227,818 and 6,264,439 and Patent Pub. No. 2007/0269322.

Although the exemplary fluid transfer device 100 illustrated in FIGS. 1-5 has proven to be a significant advance in the art, the present inventors have determined that the main check valve 107 is susceptible to improvement. For example, the surfaces of the valve element head 166 and valve seat 168 that come into contact with one another are flat. This results in a relatively large sealing surface area, and relatively low sealing pressure. A flat on flat arrangement may, in some instances, increase the likelihood that the valve seat will be scuffed by the sharp lateral edges of the valve element head 166. The flat on flat arrangement also creates an adhesion force that adds to the threshold force required to separate the valve element head 166 from the valve seat 168.

Another issue is related to the dimensional tolerances associated with the manufacture of the valve recess 118, the main check valve housing 160, and the valve seat 168. The tolerance stack-up may, in some instances, be sufficient to cause the valve seat 168 to be over-compressed when the main check valve 107 is inserted into the fluid transfer device housing 102. Over-compression may occur when the depth of the valve recess 118 is on the small end of the tolerance range, the length of the portion of the housing 160 that is inserted into the valve recess is on the large end of tolerance range, and the thickness of the valve seat 168 is on the large end of the tolerance range. Such over-compression can create an irregular seating surface as well as cause the valve seat 168 to bulge inwardly and impinge the valve element 162. Both of these conditions can result in leakage because the valve element head 166 will tend not to seat quickly and/or seal reliably.

The tolerance stack-up may, in some instances, also be sufficient to vary the length of the valve element spring 170 to such an extent that it will impart something other than the desired amount of force to the valve element 162.

Another issue relates to the positioning of the valve seat 168 during assembly. A valve seat that is not properly centered may interfere with movement of the valve element 162, which can result in leakage because the valve element head 166 will tend not to seat quickly and/or seal reliably.

As illustrated in FIGS. 6-9, a main check valve 200 in accordance with one embodiment of a present invention includes a housing 202 and a valve element 204 that is movable relative to the housing 202. The exemplary housing 202 has a generally cylindrical fluid flow portion 206, with a fluid lumen 208 that is opened and closed by the valve element 204, and a mounting portion 210 that is used to secure the main check valve 200 to a fluid transfer device or other structure. In the illustrated embodiment, which is configured for use with a cylindrical fluid transfer device, the mounting portion 210 is disk-shaped. In other embodiments, the mounting portion 210 may be resized, reshaped or omitted altogether. The mounting portion 210 is also configured to receive an inlet tube (note tube 184 in FIGS. 1-5). More specifically, the mounting portion 210 has a recess 212, with a shoulder 214, that is configured to receive an inlet tube. A filter (not shown) may be positioned within the recess 212 between the inlet tube and the shoulder 214. The recess 212 and shoulder 214 may, alternatively, be associated with the fluid flow portion 206, or both the fluid flow portion and the mounting portion 210, in other implementations of the main check valve 200.

Turning to the exemplary valve element 204, the valve element (or “plunger”) includes a shaft 216 and a head 218. A spring retainer 220 is secured to the valve element 204. More specifically, the spring retainer 220 is secured to the end of the shaft 218 opposite the head 216 and may, for example, be press fit onto the shaft. The valve element 204 is biased to the closed position illustrated in FIGS. 6 and 9, where the head 218 engages a seal surface 222 on the housing fluid flow portion 206, by a spring 224 (e.g. a coil spring) or other suitable biasing device. One end of the spring 224 abuts a shoulder 226 on the housing fluid flow portion 206, and the other end of the spring abuts the spring retainer 220.

The valve element head 218 in the illustrated embodiment is a two-part structure that includes a relatively rigid support portion 228 and an elastomeric seal 230. As illustrated for example in FIGS. 7 and 8, the elastomeric seal 230 includes a base 232 and a raised portion 234 that protrudes from the base. The exemplary base 232 is annularly shaped and is rectangular in cross-section, while the exemplary raised portion 234 has a circular shape and is semi-circular or otherwise curved in cross-section. The inner perimeter of the raised portion 234 is larger than the outlet end 236 (FIGS. 8 and 9) of the fluid lumen 208 and, accordingly, the raised portion engages the hard seal surface 222 on the housing 202 and surrounds the fluid lumen outlet end when the valve is closed. Referring to FIG. 9, the raised portion 234 is also compressed when the valve is closed.

With respect to manufacturing and materials, the exemplary housing 202 is a machined part and suitable materials for the housing include, but are not limited to, titanium, titanium alloys, stainless steel (e.g. 316L stainless steel), cobalt-nickel alloys, and refractory metals such as tantalum. The valve element 204 (less the seal 230) may also be machined and suitable materials for the machined valve element include, but are not limited to, those described above in the context of the housing 202. Alternatively, the valve element 204 (less the seal 230) may be molded. Suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the elastomeric seal 230 include, but are not limited to, elastomers such as silicone rubber, latex rubber, urethane, butyl rubber, and isoprene. The elastomeric seal 230 may be molded directly onto the relatively rigid support portion 228 of the valve element head 218, i.e. the portion of the valve element head that is more rigid than the seal, by way of a co-molding (or “insert molding”) process. Here, the valve element 204 (less the seal 230) is clamped into a mold that includes a cavity in the shape of the elastomeric seal 230, and the silicone rubber or other material is then injected into the cavity. A primer may be used to insure that the injected material sticks to the metal (e.g. titanium) or previously molded material. Such co-molding is advantageous because it eliminates the tolerance stack-up that is associated with the assembly of separately manufactured parts. Nevertheless, the elastomeric seal 230 may, if so desired, be separately manufactured and secured to the relatively rigid support portion 228 of the valve element head 218 with adhesive.

The exemplary main check valve 200 may be incorporated into a variety of fluid transfer devices. One such device is the fluid transfer device 100a illustrated in FIG. 10. The fluid transfer device 100a is essentially identical to the fluid transfer device 100, but for the substitution of main check valve 200 for main check valve 107, and similar elements are represented by similar reference numerals.

There are a variety of advantages associated with the main check valve 200. For example, the raised portion 234 on the elastomeric seal 230 reduces the contact area between the seal and the hard, flat seal surface 222 on the housing, which in turn increases the sealing pressure, as compared to a sealing arrangement that has two flat surfaces. Additionally, although the raised portion 234 will flatten slightly (FIG. 9) when the main check valve 200 is closed, the curved raised portion will reduce the amount of flat on flat surface area as well as the adhesion force associated therewith, as compared to the main check valve illustrated in FIGS. 1-5.

The main check valve 200 also avoids the above-described valve seat over-compression problems described by eliminating the valve seat. Additionally, and as illustrated in FIG. 10, there is no assembly-related over-compression of the elastomeric seal 230 when the exemplary valve 200 is inserted, for example, into the housing 102 of the fluid transfer device 100a. The only force applied to the elastomeric seal 230 is the compression force that is associated with the spring 224. The fact that the elastomeric seal 230 is in compression, as opposed to tension, is advantageous because silicone and other elastomeric materials are less likely to tear under compression. The aforementioned valve seat scuffing issue, which is associated with sharp edges on valve elements, is also eliminated.

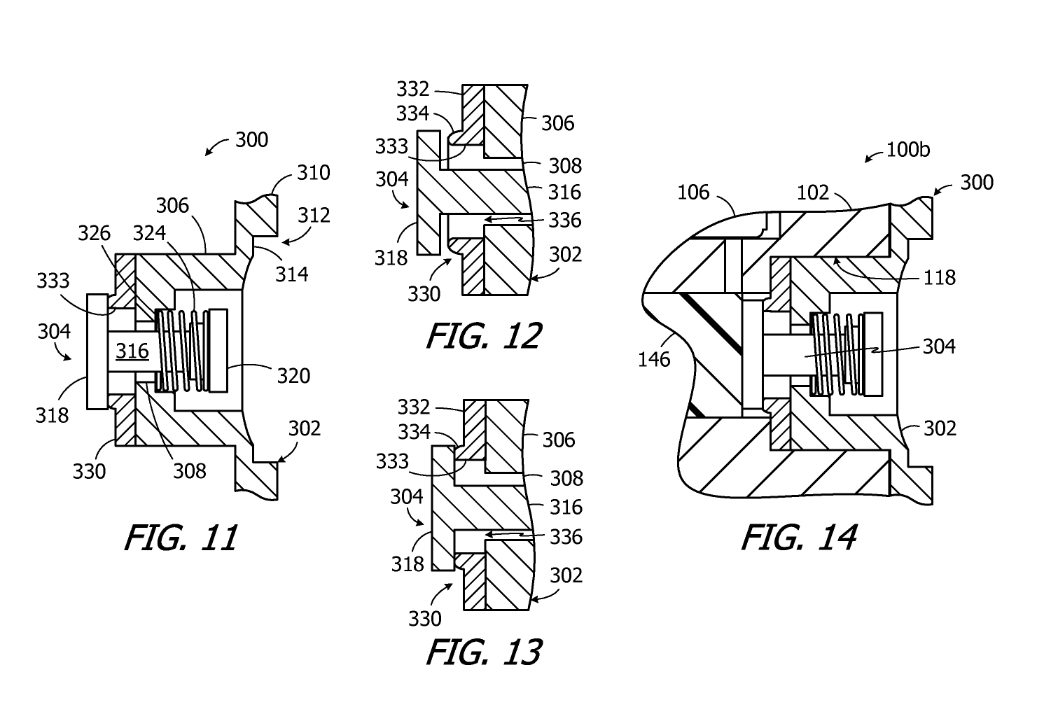

A main check valve in accordance with one embodiment of a present invention is generally represented by reference numeral 300 in FIG. 11. The exemplary main check valve 300 may be incorporated into a variety of fluid transfer devices. One such device is the fluid transfer device 100b illustrated in FIG. 14. The fluid transfer device 100b is essentially identical to the fluid transfer device 100, but for the substitution of main check valve 300 for main check valve 107, and similar elements are represented by similar reference numerals.

As illustrated in FIG. 11, the exemplary main check valve 300 includes a housing 302 and a valve element 304 that is movable relative to the housing 302. The exemplary housing 302 has a generally cylindrical fluid flow portion 306, with a fluid lumen 308 that is opened and closed by the valve element 304, and a mounting portion 310 that is used to secure the main check valve 300 to a fluid transfer device or other structure. In the illustrated embodiment, which is configured for use with a cylindrical fluid transfer device, the mounting portion 310 is disk-shaped. In other embodiments, the mounting portion 310 may be resized, reshaped or omitted altogether. The mounting portion 310 is also configured to receive an inlet tube (note tube 184 in FIGS. 1-5) and, accordingly, has a recess 312 with a shoulder 314 that is configured to receive an inlet tube. A filter (not shown) may be positioned within the recess 312 between the inlet tube and the shoulder 314. The recess 312 and shoulder 314 may, alternatively, be associated with the fluid flow portion 306, or both the fluid flow portion and the mounting portion 310, in other implementations of the main check valve 300.

The exemplary valve element 304 (or “plunger”) includes a shaft 316 and a head 318. A spring retainer 320 is secured to the valve element 304. More specifically, the spring retainer 320 is secured to the end of the shaft 316 opposite the head 318 and may, for example, be press fit onto the shaft. The valve element 304 is biased to the closed position illustrated in FIGS. 11 and 13 by a spring 324 (e.g. a coil spring) or other suitable biasing device. One end of the spring 324 abuts a shoulder 326 on the housing fluid flow portion 306, and the other end of the spring abuts the spring retainer 320.

Referring to FIGS. 12 and 13, an elastomeric valve seat 330 is positioned between the valve element head 318 and the end of the housing fluid flow portion 306. The exemplary valve seat 330 includes a base 332, which has a central opening 333 through which fluid passes, and a raised seal 334 that protrudes from the base. The exemplary base 332 is annularly shaped and is rectangular in cross-section, while the exemplary raised seal 334 has a circular shape and is semi-circular or otherwise curved in cross-section. The diameter of the base 332 is also substantially equal to that of the housing cylindrical fluid flow portion 306. The raised seal 334 extends along the inner diameter of the base 332. The valve element head 318 engages and compresses the valve seat raised seal 334 when the valve is closed.

The fluid lumen 308 may be sized based on the desired fluid flow characteristics of the valve 300 and, in the illustrated embodiment is about 0.019 inch to about 0.035 inch in diameter. The valve seat opening 333 will typically be at least the same size as fluid lumen 308, and is larger in the illustrated embodiment, in order to prevent the valve seat 330 from interfering with fluid flow. Another factor associated with the size of the valve seat opening 333 is the size of the valve element shaft 316. More specifically, the opening 333 should be sized such that, in the event that the valve seat 330 is overcompressed and/or is off-center, the valve seat will be not impinge the valve element shaft 316. To that end, the exemplary valve element shaft 316 is about 0.014 inch to about 0.026 inch in diameter, and the exemplary valve seat opening 333 will be about 0.024 inch to about 0.045 inch in diameter.

With respect to manufacturing and materials, the exemplary housing 302 is a machined part and suitable materials for the housing include, but are not limited to, titanium, titanium alloys, stainless steel (e.g. 316L stainless steel), cobalt-nickel alloys, and refractory metals such as tantalum. The valve element 304 may also be machined and suitable materials for the machined valve element include, but are not limited to, those described above in the context of the housing 302. Alternatively, and as discussed in greater detail below, the valve element 304 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 330 include, but are not limited to, elastomers such as silicone rubber, latex rubber, urethane, butyl rubber, and isoprene. The valve seat 330 may be molded directly onto the housing 302 by way of a co-molding (or “insert molding”) process. Here, the housing 302 is clamped into a mold that includes a cavity in the shape of the valve seat 330, and the silicone rubber or other material is then injected into the cavity. A primer may be used to insure that the injected material sticks to the housing 302. Such co-molding is advantageous because it eliminates the tolerance stack-up that is associated with the assembly of separately manufactured parts. Nevertheless, the valve seat 330 may, if desired, be separately manufactured and secured to the housing 302 with adhesive.

There are a variety of advantages associated with the main check valve 300. For example, the valve seat raised seal 334 reduces the contact area between valve seat and the valve element head 318, which in turn increases the sealing pressure, as compared to a sealing arrangement that has two flat surfaces. Additionally, although the raised seal 334 will flatten slightly (FIG. 13) when the main check valve 300 is closed, the curved protruding ring will reduce the amount of flat on flat surface area as well as the adhesion force associated therewith, as compared to the main check valve illustrated in FIGS. 1-5. Compressing the valve seat raised seal 334 is also advantageous because silicone and other elastomeric materials tend to tear when in tension. The scuffing issue is also obviated because the sharp lateral edges of the valve element head 318 do not come into contact with the valve seat 330.

The main check valve 300 also avoids the aforementioned valve over-compression problems because, and as is illustrated in FIG. 14, although the valve seat base 332 is compressed when the main check valve is inserted into the fluid transfer device 100b, the sealing surface (i.e. the valve seat raised seal 334) is not compressed. The only force applied to the raised seal 334 is the force associated with the spring 324. Additionally, even if there is some over-compression of the valve seat base 332, the valve seat 330 will not impinge the valve element shaft 316 because the inner diameter of the base 332 is relatively large.

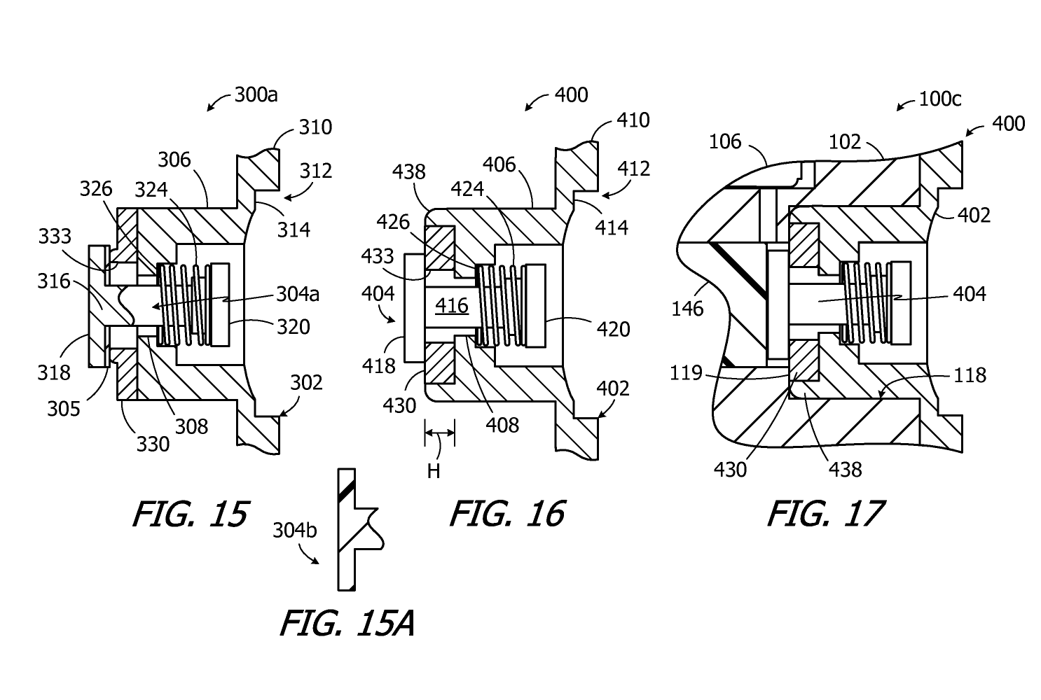

A main check valve in accordance with one embodiment of a present invention is generally represented by reference numeral 300a in FIG. 15. The exemplary main check valve 300a is essentially identical to main check valve 300 and similar elements are represented by similar reference numerals. The exemplary main check valve 300a may also be incorporated into a fluid transfer device in the same manner as the main check valve 300. Here, however, the valve element 304a is configured so as to further reduce the likelihood that the valve element will stick to the valve seat 330.

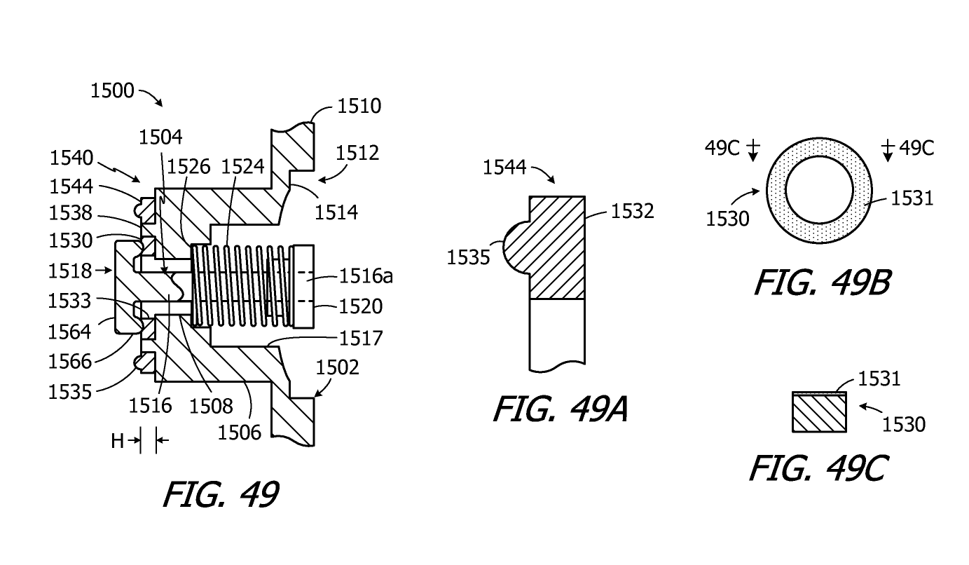

In the illustrated embodiment, the valve element 304a includes a non-stick surface 305 (or “release layer”) on the side of the head 318 that abuts the valve seat 330. The non-stick surface may, for example, be in the form of a layer of polytetrafluoroethylene (PTFE), which is commonly sold under the Teflon® trademark. Other suitable materials for the non-stick surface_include, but are not limited to, parylene and titanium nitride. The pull-off adhesion of the non-stick material should be less than about 0.5 psi in those instances where the valve seat opening is about 0.040 inch. The PTFE layer, which is about 0.0001 to 0.005 inch in the exemplary embodiment, may be molded or co-molded onto a head 318. Alternatively, the non-stick surface may be formed by simply forming the valve element head, or the entire valve element (note valve element 304b in FIG. 15A), from PTFE, acetyl or another suitable non-stick material. Still another use of non-stick material, i.e. non-stick material on the valve seat, is discussed below with reference to FIGS. 49B and 49C.

In addition to the benefits described above in the context of main check valve 300, main check valve 300a further reduces the likelihood that the valve element 304a will stick to the valve seat 330. It should also be noted here that the main check valves described above and below with reference to FIGS. 1-5 and 16-58 may also include non-stick surface that abuts the valve seat. Alternatively, the non-stick surface may be formed by simply forming the valve element heads, or the valve element heads and shafts, from a non-stick material. With respect to the main check valves illustrated in FIGS. 6-10 and 59-62, a layer of non-stick material may be formed on the hard surface of the housing that extends around the fluid lumen outlet and comes into contact with the valve element.

A main check valve in accordance with one embodiment of a present invention is generally represented by reference numeral 400 in FIG. 16. The exemplary main check valve 400 may be incorporated into a variety of fluid transfer devices. One such device is the fluid transfer device 100c illustrated in FIG. 17. The fluid transfer device 100c is essentially identical to the fluid transfer device 100, but for the substitution of main check valve 400 for main check valve 107, and similar elements are represented by similar reference numerals.

As illustrated for example in FIG. 16, the main check valve 400 includes a housing 402 and a valve element 404 that is movable relative to the housing 402. The exemplary housing 402 has a generally cylindrical fluid flow portion 406, with a fluid lumen 408 that is opened and closed by the valve element 404, and a mounting portion 410 that is used to secure the main check valve 400 to a fluid transfer device or other structure. In the illustrated embodiment, which is configured for use with a cylindrical fluid transfer device, the mounting portion 410 is disk-shaped. In other embodiments, the mounting portion 410 may be resized, reshaped or omitted altogether. The mounting portion 410 is also configured to receive an inlet tube (note tube 184 in FIGS. 1-5) and, accordingly, has a recess 412, with a shoulder 414 that is configured to receive the inlet tube. A filter (not shown) may be positioned within the recess 412 between the inlet tube and the shoulder 414. The recess 412 and shoulder 414 may, alternatively, be associated with the fluid flow portion 406, or both the fluid flow portion and the mounting portion 410, in other implementations of the main check valve 400.

The exemplary valve element 404 (or “plunger”) includes a shaft 416 and a head 418. A spring retainer 420 is secured to the valve element 404. More specifically, the spring retainer 420 is secured to the end of the shaft 416 opposite the head 418 and may, for example, be press fit onto the shaft. The valve element 404 is biased to the closed position illustrated in FIG. 16 by a spring 424 (e.g. a coil spring) or other suitable biasing device. One end of the spring 424 abuts a shoulder 426 on the housing fluid flow portion 406, and the other end of the spring abuts the spring retainer 420.

An elastomeric valve seat 430 is positioned between the valve element head 418 and the end of the housing fluid flow portion 406. The exemplary valve seat 430 has a generally annular shape and a central opening 433 through which fluid passes. The fluid lumen 408 may be sized based on the desired fluid flow characteristics of the valve 400 and, in the illustrated embodiment is about 0.019 inch to about 0.035 inch in diameter. The valve seat opening 433 will typically be at least the same size as fluid lumen 308, and is larger in the illustrated embodiment, in order to prevent the valve seat 430 from interfering with fluid flow. Another factor associated with the size of the valve seat opening 433 is the size of the valve element shaft 416. More specifically, the opening 433 should be sized such that, in the event that the valve seat 430 is overcompressed and/or is off-center, the valve seat will be not impinge the valve element shaft 416. To that end, the exemplary valve element shaft 416 is about 0.014 inch to about 0.026 inch in diameter, and the exemplary valve seat opening 433 will be about 0.024 inch to about 0.045 inch in diameter.

The exemplary main check valve 400 illustrated in FIG. 16 is also provided with apparatus that prevents over-compression of the elastomeric valve seat 430. More specifically, the housing 402 includes a stop member 438 that extends from the end of flow portion 406. The exemplary stop member 438 is a rigid, substantially annular structure that is integral with the flow portion 406 and has a height H that is equal to, or is slightly less than (e.g. about 0.003 inch less than), the thickness of the elastomeric valve seat 430. The stop member 438, which is located outwardly of the valve seat 430, limits movement of the main check valve 400 relative to the structure into which the main check valve is inserted. More specifically, in the exemplary fluid transfer device 100c illustrated in FIG. 17, the stop member 438 engages the end wall 119 of the valve recess 118. It should also be noted that the stop member 438 may define a continuous perimeter (as shown) or, alternatively, be composed of a plurality of spaced elements that together define a discontinuous perimeter. The stop member 438, whether continuous or discontinuous, may also have an overall shape other than the illustrated substantially annular shape.

With respect to manufacturing and materials, the exemplary housing 402 is a machined part and suitable materials for the housing include, but are not limited to, titanium, titanium alloys, stainless steel (e.g. 316L stainless steel), cobalt-nickel alloys, and refractory metals such as tantalum. The valve element 404 may also be machined and suitable materials for the machined valve element include, but are not limited to, those described above in the context of the housing 402. Alternatively, and as discussed in greater detail below, the valve element 404 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 430 include, but are not limited to, elastomers such as silicone rubber, latex rubber, urethane, butyl rubber, and isoprene. The valve seat 430 may be molded directly onto the housing 402 by way of a co-molding (or “insert molding”) process. Here, the housing 402 is clamped into a mold that includes a cavity in the shape of the valve seat 430, and the silicone rubber or other material is then injected into the cavity. A primer may be used to insure that the injected material sticks to the housing 402. Such co-molding is advantageous because it eliminates the tolerance stack-up that is associated with the assembly of separately manufactured parts. Nevertheless, the valve seat 430 may, if desired, be separately manufactured and secured to the housing 402 with adhesive.

There are a variety of advantages associated with the main check valve 400. For example, compression of the valve seat 430 in the exemplary main check valve 400 is a function of only two dimensions, i.e. the thickness of the valve seat and the height H of the stop member 438, as compared to the three dimensions associated with the main check valve 107 illustrated in FIGS. 1-5. It is, therefore, substantially less likely that a tolerance stack-up will result in over-compression of the valve seat 430, as compared to the main check valve 107. Additionally, even if there is some over-compression of the valve seat 430, the valve seat will not impinge the valve element shaft 416 because the diameter of the opening 433 is relatively large.

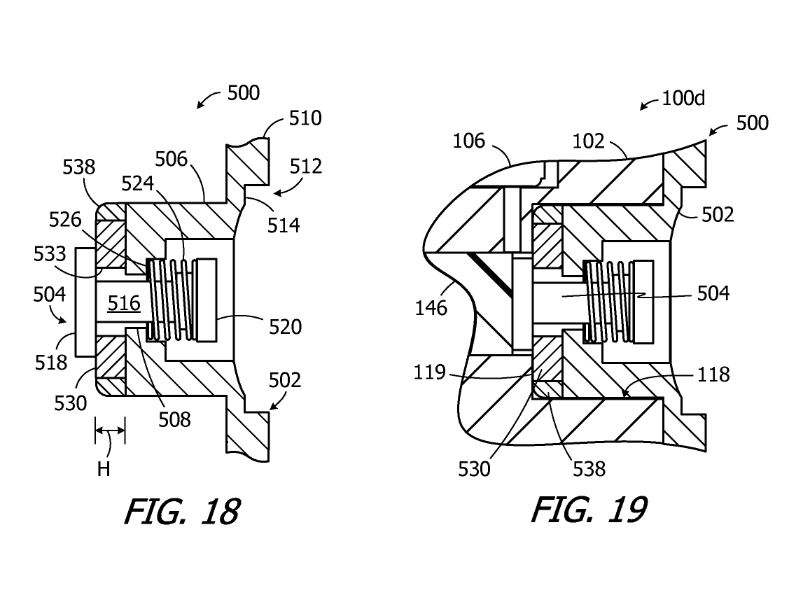

A main check valve in accordance with one embodiment of a present invention is generally represented by reference numeral 500 in FIG. 18. The exemplary main check valve 500 may be incorporated into a variety of fluid transfer devices. One such device is the fluid transfer device 100d illustrated in FIG. 19. The fluid transfer device 100d is essentially identical to the fluid transfer device 100, but for the substitution of main check valve 500 for main check valve 107, and similar elements are represented by similar reference numerals.

As illustrated for example in FIG. 18, the main check valve 500 includes a housing 502 and a valve element 504 that is movable relative to the housing 502. The exemplary housing 502 has a generally cylindrical fluid flow portion 506, with a fluid lumen 508 that is opened and closed by the valve element 504, and a mounting portion 510 that is used to secure the main check valve 500 to a fluid transfer device or other structure. In the illustrated embodiment, which is configured for use with a cylindrical fluid transfer device, the mounting portion 510 is disk-shaped. In other embodiments, the mounting portion 510 may be resized, reshaped or omitted altogether. The mounting portion 510 is also configured to receive an inlet tube (note tube 184 in FIGS. 1-5) and, accordingly, has a recess 512, with a shoulder 514 that is configured to receive the inlet tube. A filter (not shown) may be positioned within the recess 512 between the inlet tube and the shoulder 514. The recess 512 and shoulder 514 may, alternatively, be associated with the fluid flow portion 506, or both the fluid flow portion and the mounting portion 510, in other implementations of the main check valve 500.

The exemplary valve element 504 (or “plunger”) includes a shaft 516 and a head 518. A spring retainer 520 is secured to the valve element 504. More specifically, the spring retainer 520 is secured to the end of the shaft 516 opposite the head 518 and may, for example, be press fit onto the shaft. The valve element 504 is biased to the closed position illustrated in FIG. 18 by a spring 524 (e.g. a coil spring) or other suitable biasing device. One end of the spring 524 abuts a shoulder 526 on the housing fluid flow portion 506, and the other end of the spring abuts the spring retainer 520.

An elastomeric valve seat 530 is positioned between the valve element head 518 and the end of the housing fluid flow portion 506. The exemplary valve seat 530 has a generally annular shape and a central opening 533 through which fluid passes. The fluid lumen 508 may be sized based on the desired fluid flow characteristics of the valve 500 and, in the illustrated embodiment is about 0.019 inch to about 0.035 inch in diameter. The valve seat opening 533 will typically be at least the same size as fluid lumen 508, and is larger in the illustrated embodiment, in order to prevent the valve seat 530 from interfering with fluid flow. Another factor associated with the size of the valve seat opening 533 is the size of the valve element shaft 516. More specifically, the opening 533 should be sized such that, in the event that the valve seat 530 is overcompressed and/or is off-center, the valve seat will be not impinge the valve element shaft 516. To that end, the exemplary valve element shaft 516 is about 0.014 inch to about 0.026 inch in diameter, and the exemplary valve seat opening 533 will be about 0.024 inch to about 0.045 inch in diameter.

The exemplary main check valve 500 illustrated in FIG. 18 is also provided with apparatus that prevents over-compression of the elastomeric valve seat 530. More specifically, the main check valve 500 includes a stop member 538 that abuts the housing fluid flow portion 506 and extends around the valve seat 530. The exemplary stop member 538 is a rigid, substantially annular structure that has a height H which is equal to, or is slightly less than (e.g. about 0.003 inch less than), the thickness of the valve seat 530. The stop member 538, which is located outwardly of the valve seat 530, limits movement of the main check valve 500 relative to the structure into which the main check valve is inserted. More specifically, in the exemplary fluid transfer device 100d illustrated in FIG. 19, the stop member 538 engages the end wall 119 of the valve recess 118. It should also be noted that the stop member 538 may define a continuous perimeter (as shown) or, alternatively, be composed of a plurality of spaced elements that together define a discontinuous perimeter. The stop member 538, whether continuous or discontinuous, may also have an overall shape other than the illustrated substantially annular shape.

With respect to manufacturing and materials, the exemplary housing 502 and separate stop member 538 are machined parts and suitable materials for the housing and stop member include, but are not limited to, titanium, titanium alloys, stainless steel (e.g. 316L stainless steel), cobalt-nickel alloys, and refractory metals such as tantalum. The valve element 504 may also be machined and suitable materials for the machined valve element include, but are not limited to, those described above in the context of the housing 502. Alternatively, as discussed in greater detail below, the valve element 504 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 530 include, but are not limited to, elastomers such as silicone rubber, latex rubber, urethane, butyl rubber, and isoprene. The valve seat 530 may be molded directly onto the stop member 538 by way of a co-molding (or “insert molding”) process. Here, the stop member 538 is clamped into a mold that includes a cavity in the shape of the valve seat 530, and the silicone rubber or other material is then injected into the cavity. A primer may be used to insure that the injected material sticks to the stop member 538. Such co-molding is advantageous because it eliminates the tolerance stack-up that is associated with the assembly of separately manufactured parts. Nevertheless, the valve seat 530 may, alternatively, be separately manufactured and, if desired, secured to the stop member 538.

There are a variety of advantages associated with the main check valve 500. For example, compression of the valve seat 530 in the exemplary main check valve 500 is a function of only two dimensions, i.e. the thickness of the valve seat and the height H of the stop member 538, as compared to the three dimensions associated with the main check valve 107 illustrated in FIGS. 1-5. It is, therefore, substantially less likely that a tolerance stack-up will result in over-compression of the valve seat 530, as compared to the main check valve 107. Additionally, even if there is some over-compression of the valve seat annular portion 530, the valve seat 530 will not impinge the valve element shaft 516 because the diameter of the opening 533 is relatively large.

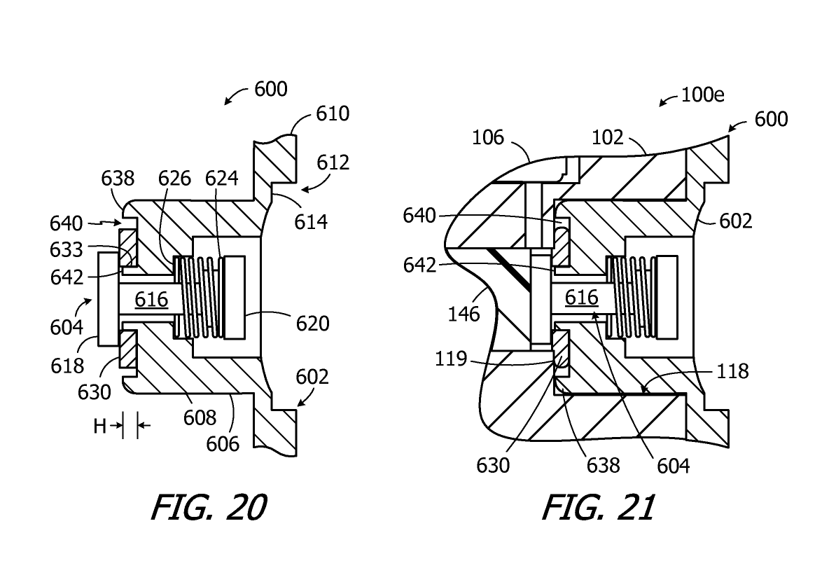

A main check valve in accordance with one embodiment of a present invention is generally represented by reference numeral 600 in FIG. 20. The exemplary main check valve 600 may be incorporated into a variety of fluid transfer devices. One such device is the fluid transfer device 100e illustrated in FIG. 21. The fluid transfer device 100e is essentially identical to the fluid transfer device 100, but for the substitution of main check valve 600 for main check valve 107, and similar elements are represented by similar reference numerals.

As illustrated for example in FIG. 20, the main check valve 600 includes a housing 602 and a valve element 604 that is movable relative to the housing 602. The exemplary housing 602 has a generally cylindrical fluid flow portion 606, with a fluid lumen 608 that is opened and closed by the valve element 604, and a mounting portion 610 that is used to secure the main check valve 600 to a fluid transfer device or other structure. In the illustrated embodiment, which is configured for use with a cylindrical fluid transfer device, the mounting portion 610 is disk-shaped. In other embodiments, the mounting portion 610 may be resized, reshaped or omitted altogether. The mounting portion 610 is also configured to receive an inlet tube (note tube 184 in FIGS. 1-5) and, accordingly, has a recess 612, with a shoulder 614 that is configured to receive the inlet tube. A filter (not shown) may be positioned within the recess 612 between the inlet tube and the shoulder 614. The recess 612 and shoulder 614 may, alternatively, be associated with the fluid flow portion 606, or both the fluid flow portion and the mounting portion 610, in other implementations of the main check valve 600.

The exemplary valve element 604 (or “plunger”) includes a shaft 616 and a head 618. A spring retainer 620 is secured to the valve element 604. More specifically, the spring retainer 620 is secured to the end of the shaft 616 opposite the head 618 and may, for example, be press fit onto the shaft. The valve element 604 is biased to the closed position illustrated in FIG. 20 by a spring 624 (e.g. a coil spring) or other suitable biasing device. One end of the spring 624 abuts a shoulder 626 on the housing fluid flow portion 606, and the other end of the spring abuts the spring retainer 620. An elastomeric valve seat 630, which has a generally annular shape, is positioned between the valve element head 618 and the end of the housing fluid flow portion 606.

The exemplary main check valve 600 illustrated in FIG. 20 is provided with apparatus that prevents over-compression of the elastomeric valve seat 630. More specifically, the housing 602 includes a stop member 638 that extends from the end of flow portion 606. The exemplary stop member 638 is a rigid, substantially annular structure that is integral with the flow portion 606 and has a height H that is equal to, or is slightly less than (e.g. about 0.003 inch less than), the thickness of the elastomeric valve seat 630. The stop member 638, which is located outwardly of the valve seat 630, limits movement of the main check valve 600 relative to the structure into which the main check valve is inserted. More specifically, in the exemplary fluid transfer device 100e illustrated in FIG. 21, the stop member 638 engages the end wall 119 of the valve recess 118. It should also be noted that the stop member 638 may define a continuous perimeter (as shown) or, alternatively, be composed of a plurality of spaced elements that together define a discontinuous perimeter. The stop member 638, whether continuous or discontinuous, may also have an overall shape other than the illustrated substantially annular shape.

The outer diameter of valve seat 630 is slightly less (e.g. about 0.005 inch to 0.020 inch in the exemplary embodiment) than the inner diameter of the stop member 638. This difference produces a gap 640 that allows the valve seat 630 to deform (or “extrude”) outwardly when compressed in the manner illustrated in FIG. 21.

The exemplary main check valve 600 illustrated in FIG. 20 is further provided with apparatus that prevents the elastomeric valve seat 630 from deforming (or “extruding”) inwardly. In the illustrated embodiment, the housing 602 includes an inner wall 642 that is positioned between the fluid lumen 608 and the valve seat 630 and separates the inner surface of the valve seat (i.e. the surface that defines the opening 633) from the fluid lumen. The exemplary inner wall 642 has inner diameter that is equal to the diameter of the lumen 608 and an outer diameter that is equal to the inner diameter of the valve seat 630. The inner surface of the inner wall 642 also defines a portion of the fluid lumen 608.

The inner wall may, alternatively, be a separate structural element from the housing. For example, the inner wall may be in the form of a relatively rigid ring onto which the valve seat is molded.

With respect to manufacturing and materials, the exemplary housing 602 is a machined part and suitable materials for the housing include, but are not limited to, titanium, titanium alloys, stainless steel (e.g. 316L stainless steel), cobalt-nickel alloys, and refractory metals such as tantalum. The valve element 604 may also be machined and suitable materials for the machined valve element include, but are not limited to, those described above in the context of the housing 502. Alternatively, as discussed in greater detail below, the valve element 604 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 630 include, but are not limited to, elastomers such as silicone rubber, latex rubber, urethane, butyl rubber, and isoprene. The valve seat 630 may be molded directly onto the housing 602 by way of a co-molding (or “insert molding”) process. Here, the housing 602 is clamped into a mold that includes a cavity in the shape of the valve seat 630, and the silicone rubber or other material is then injected into the cavity. A primer may be used to insure that the injected material sticks to the housing 602. Such co-molding is advantageous because it eliminates the tolerance stack-up that is associated with the assembly of separately manufactured parts. Nevertheless, the valve seat 630 may, alternatively, be separately manufactured, positioned around the inner wall 642 and, if desired, secured to the housing 602 with adhesive.

There are a variety of advantages associated with the main check valve 600. For example, compression of the valve seat 630 in the exemplary main check valve 600 is a function of only two dimensions, i.e. the thickness of the valve seat and the height H of the stop member 638, as compared to the three dimensions associated with the main check valve 107 illustrated in FIGS. 1-5. It is, therefore, substantially less likely that a tolerance stack-up will result in over-compression of the valve seat 630, as compared to the main check valve 107. Additionally, even if there is some compression of the valve seat 630, the inner wall 642 will prevent the valve seat from impinging the valve element shaft 616, while the gap 640 will allow the valve seat 630 to deform outwardly, thereby preventing the formation of an irregular sealing surface. The inner wall 642 also centers the valve seat 630.

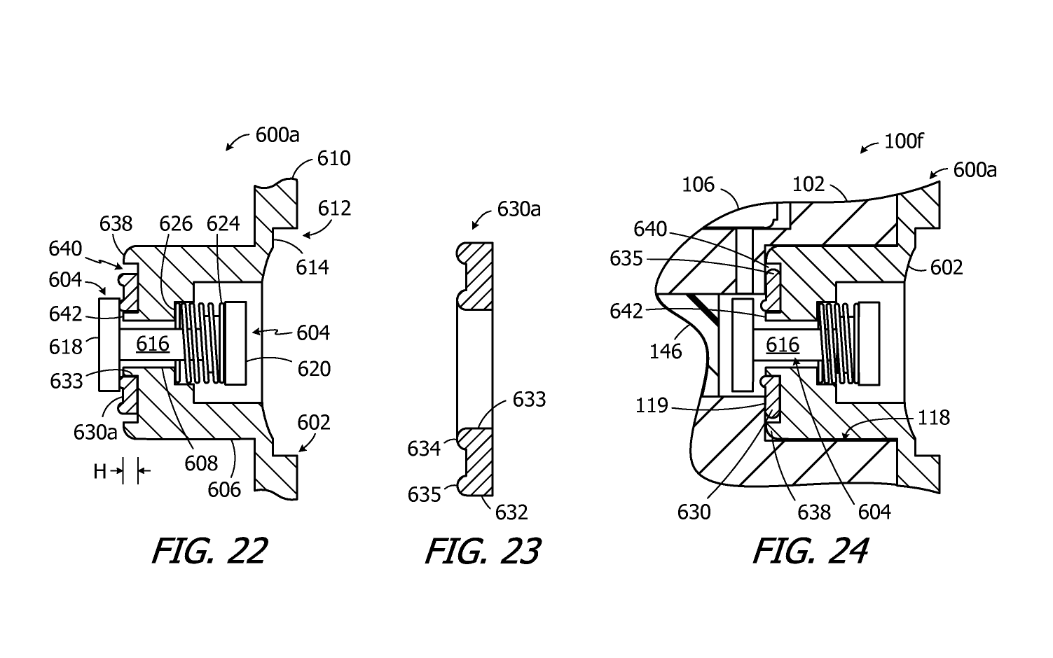

A main check valve in accordance with one embodiment of a present invention is generally represented by reference numeral 600a in FIG. 22. The exemplary main check valve 600a is essentially identical to main check valve 600 and similar elements are represented by similar reference numerals. The manufacturing and materials described above with reference to main check valve 600 are also applicable to main check valve 600a. The exemplary main check valve 600a may also be incorporated into a variety of fluid transfer devices. One such device is the fluid transfer device 100f illustrated in FIG. 24. The fluid transfer device 100f is essentially identical to the fluid transfer device 100, but for the substitution of main check valve 600a for main check valve 107, and similar elements are represented by similar reference numerals.

Here, however, the exemplary valve seat 630a has a base 632, with a central opening 633 through which fluid passes, an inner raised seal 634 that protrudes from the base, and an outer raised seal 635 that protrudes from the base, as is shown in FIG. 23. The exemplary base 632 is annularly shaped and is rectangular in cross-section, while exemplary raised seals 634 and 635 have circular shapes and are semi-circular or otherwise curved in cross-section. The raised seal 634 is aligned with the inner diameter of the base 632, and the raised seal 635 is aligned with the outer diameter of the base. The valve element head 618 engages and compresses the inner raised seal 634 when the valve is closed. Additionally, the outer diameter of valve seat 630a is slightly less (e.g. about 0.005 inch to 0.020 inch) than the inner diameter of the stop member 638. This results in a gap 640 into which the valve seat 630a can deform (or “extrude”) when compressed in the manner illustrated in FIG. 24.

In addition to the benefits described above in the context of main check valve 600, the valve seat inner raised seal 634 in the main check valve 600a reduces the contact area between valve seat and the valve element head 618, which in turn increases the sealing pressure, as compared to a sealing arrangement that has two flat surfaces. Additionally, although the raised seal 634 will flatten slightly (FIG. 22) when the main check valve 600 is closed, the curved protruding ring will reduce the amount of flat on flat surface area as well as the adhesion force associated therewith, as compared to the main check valve illustrated in FIGS. 1-5. Compressing the valve seat raised seal 634 is also advantageous because silicone and other elastomeric materials tend to tear when in tension. The scuffing issue is also obviated because the sharp lateral edges of the valve element head 618 do not come into contact with the valve seat 630.

Turning to the outer raised seal 635 of the valve seat 630a, the outer raised seal will be under compression when the main check valve 600a is inserted into the valve recess 118 of the housing 102. Compressing the raised seal 635 is advantageous because silicone and other elastomeric materials tend to tear when in tension. The raised seal 635 also increases sealing pressure without increasing the size of entire valve seat and does so in a manner that is relatively easy to manufacture.

It should also be noted that the valve seats with inner and outer raised seals, i.e. valve seats similar to exemplary valve seat 630a, may be used in combination with other valves that do not include both a stop member and a housing inner wall that is aligned with the valve seat. By way of example, but not limitation, such a valve seat may be included in the main check valves 300, 300a, 400 and 500 described above, and the main check valves 900, 1000, 1100, 1200 and 1300 described below.

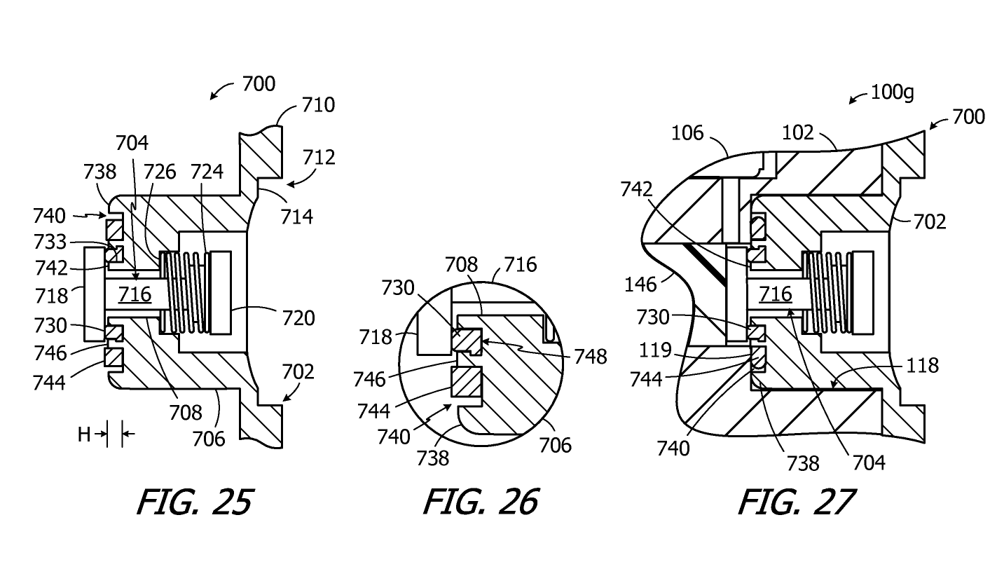

A main check valve in accordance with one embodiment of a present invention is generally represented by reference numeral 700 in FIG. 25. The exemplary main check valve 700 may be incorporated into a variety of fluid transfer devices. One such device is the fluid transfer device 100g illustrated in FIG. 27. The fluid transfer device 100g is essentially identical to the fluid transfer device 100, but for the substitution of main check valve 700 for main check valve 107, and similar elements are represented by similar reference numerals.

As illustrated for example in FIG. 25, the main check valve 700 includes a housing 702 and a valve element 704 that is movable relative to the housing 702. The exemplary housing 702 has a generally cylindrical fluid flow portion 706, with a fluid lumen 708 that is opened and closed by the valve element 704, and a mounting portion 710 that is used to secure the main check valve 700 to a fluid transfer device or other structure. In the illustrated embodiment, which is configured for use with a cylindrical fluid transfer device, the mounting portion 710 is disk-shaped. In other embodiments, the mounting portion 710 may be resized, reshaped or omitted altogether. The mounting portion 710 is also configured to receive an inlet tube (note tube 184 in FIGS. 1-5) and, accordingly, has a recess 712, with a shoulder 714 that is configured to receive the inlet tube. A filter (not shown) may be positioned within the recess 712 between the inlet tube and the shoulder 714. The recess 712 and shoulder 714 may, alternatively, be associated with the fluid flow portion 706, or both the fluid flow portion and the mounting portion 710, in other implementations of the main check valve 700.

The exemplary valve element 704 (or “plunger”) includes a shaft 716 and a head 718. A spring retainer 720 is secured to the valve element 704. More specifically, the spring retainer 720 is secured to the end of the shaft 716 opposite the head 718 and may, for example, be press fit onto the shaft. The spring retainer 720 may, for example, be press fit onto the shaft 716. The valve element 704 is biased to the closed position illustrated in FIG. 25 by a spring 724 (e.g. a coil spring) or other suitable biasing device. One end of the spring 724 abuts a shoulder 726 on the housing fluid flow portion 706, and the other end of the spring abuts the spring retainer 720.

An elastomeric valve seat 730 is positioned between the valve element head 718 and the end of the housing fluid flow portion 706. The valve seat 730 is sized and shaped such that it will be engaged by the valve element head 718, but not by the housing of the associated fluid transfer device, e.g. the fluid transfer device 100g. The valve seat 730 is generally annular in shape and the portion that engages the valve element heat 718 is semi-circular or otherwise curved in cross-section. Referring to FIG. 26, the exemplary housing fluid flow portion 706 includes an L-shaped valve seat retainer 746 that, together with the inner wall 742 (discussed below), forms a valve seat slot 748. The respective shapes of the valve seat 730 and the valve seat slot 748 create a mechanical interlock that secures the valve seat to the housing fluid flow portion 706.

An elastomeric gasket 744, which is a separate structural element from the valve seat 730, may be used to form a seal between the housing fluid flow portion 706 and the housing 102 or other structure into which the main check valve 700 is inserted. The outer diameter of the exemplary gasket 744 is slightly less (e.g. about 0.005 inch to about 0.020 inch in the exemplary embodiment) than the inner diameter of the stop member 738 (discussed below). This difference produces a gap 740 which allows the gasket 744 to deform (or “extrude”) outwardly when compressed in the manner illustrated in FIG. 27.

The exemplary main check valve 700 illustrated in FIG. 25 is provided with apparatus that prevents the elastomeric valve seat 730 from deforming (or “extruding”) inwardly. In the illustrated embodiment, the housing 702 includes an inner wall 742 that is positioned between the fluid lumen 708 and the valve seat 730 and separates the inner surface of the valve seat (i.e. the surface that defines the opening 733) from the fluid lumen. The exemplary inner wall 742 has inner diameter that is equal to the diameter of the fluid lumen 708 and an outer diameter that is equal to the inner diameter of the valve seat 730. The inner surface of the inner wall 742 also defines a portion of the fluid lumen 708.

The inner wall may, alternatively, be a separate structural element from the housing. For example, the inner wall may be in the form of a relatively rigid ring onto which the valve seat is molded.

The exemplary main check valve 700 illustrated in FIG. 25 is also provided with apparatus that controls the compression of the gasket 744. More specifically, the housing 702 includes a stop member 738 that extends from the end of flow portion 706. The exemplary stop member 738 is a rigid, substantially annular structure that is integral with the flow portion 706 and has a height H that is equal to, or is slightly less than (e.g. about 0.003 inch less than), the thickness of the gasket. The stop member 738, which is located outwardly of the valve seat 730 and gasket 744, limits movement of the main check valve 700 relative to the structure into which the main check valve is inserted. More specifically, in the exemplary fluid transfer device 100g illustrated in FIG. 27, the stop member 738 engages the end wall 119 of the valve recess 118. It should also be noted that the stop member 738 may define a continuous perimeter (as shown) or, alternatively, be composed of a plurality of spaced elements that together define a discontinuous perimeter. The stop member 738, whether continuous or discontinuous, may also have an overall shape other than the illustrated substantially annular shape.

With respect to manufacturing and materials, the exemplary housing 702 is a machined or fabricated part and suitable materials for the housing include, but are not limited to, titanium, titanium alloys, stainless steel (e.g. 316L stainless steel), cobalt-nickel alloys, and refractory metals such as tantalum. The valve element 704 may also be machined and suitable materials for the machined valve element include, but are not limited to, those described above in the context of the housing 702. Alternatively, as discussed in greater detail below, the valve element 704 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 730 and gasket 744 include, but are not limited to, elastomers such as silicone rubber, latex rubber, urethane, butyl rubber, and isoprene. The valve seat 730 and gasket 744 may be molded directly onto the housing 702 by way of a co-molding (or “insert molding”) process. Here, the housing 702 is clamped into a mold that includes cavities in the shape of the valve seat 730 and gasket 744, and the silicone rubber or other material is then injected into the cavities. A primer may be used to insure that the injected material sticks to the housing 702. Such co-molding is advantageous because it eliminates the tolerance stack-up that is associated with the assembly of separately manufactured parts. Nevertheless, the valve seat 730 may, alternatively, be separately manufactured and snap-fit into the L-shaped slot defined by the valve seat retainer 746. The gasket 744 may, alternatively, be separately manufactured, positioned around the valve seat retainer 746 and, if desired, secured to the housing 702 with adhesive.

There are a variety of advantages associated with the main check valve 700. For example, the main check valve 700 avoids the above-described valve seat over-compression problems by configuring the valve seat such that it does not engage the housing of the fluid transfer device into which the valve is inserted. More specifically, as illustrated in FIG. 27, there is no assembly-related compression of the valve seat 730 when the exemplary valve 700 is inserted into the fluid transfer device housing 102. The only force applied to the valve seat 730 is that associated with the spring 724. Even if there is some over-compression of the valve seat 730, the inner wall 742 will prevent the valve seat from impinging the valve element shaft 716.

Additionally, because the portion of the valve seat 730 that engages the valve element heat 718 is semi-circular or otherwise curved in cross-section, the contact area between valve seat and the valve element head 718 is decreased, which in turn increases the sealing pressure, as compared to a sealing arrangement that has two flat surfaces. Although the valve seat 730 will flatten slightly (FIG. 25) when the main check valve 700 is closed, the curved portion will reduce the amount of flat on flat surface area as well as the adhesion force associated therewith, as compared to the main check valve illustrated in FIGS. 1-5. Compressing the valve seat 730 is also advantageous because silicone and other elastomeric materials tend to tear when in tension. The scuffing issue is also obviated because the sharp lateral edges of the valve element head 718 do not come into contact with the valve seat 730. Moreover, molding the valve seat 730 into housing 702 insures that the valve seat will be properly centered.

Turning to the gasket 744, compression of the gasket is a function of only two dimensions, i.e. the thickness of the gasket and the height H of the stop member 738, as compared to the three dimensions associated with the main check valve 107 illustrated in FIGS. 1-5. It is, therefore, substantially less likely that a tolerance stack-up will result in over-compression of the gasket 744, as compared to the valve seat in the main check valve 107 which also functions as a gasket. The gap 740 also allows the gasket 744 to deform outwardly when compressed, thereby reducing the likelihood of an irregular sealing surface. Moreover, even in those instances where the gasket 744 is over-compressed during assembly, the over-compression will not reduce the effectiveness of the valve seat 730 because the valve seat is not connected to the gasket.

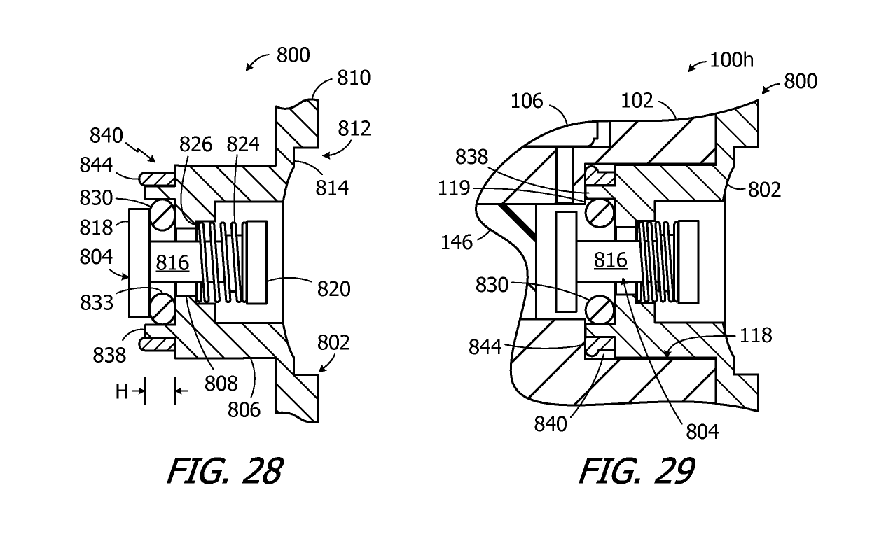

A main check valve in accordance with one embodiment of a present invention is generally represented by reference numeral 800 in FIG. 28. The exemplary main check valve 800 may be incorporated into a variety of fluid transfer devices. One such device is the fluid transfer device 100h illustrated in FIG. 29. The fluid transfer device 100h is essentially identical to the fluid transfer device 100, but for the substitution of main check valve 800 for main check valve 107, and similar elements are represented by similar reference numerals.

As illustrated for example in FIG. 28, the main check valve 800 includes a housing 802 and a valve element 804 that is movable relative to the housing 802. The exemplary housing 802 has a generally cylindrical fluid flow portion 806, with a fluid lumen 808 that is opened and closed by the valve element 804, and a mounting portion 810 that is used to secure the main check valve 800 to a fluid transfer device or other structure. In the illustrated embodiment, which is configured for use with a cylindrical fluid transfer device, the mounting portion 810 is disk-shaped. In other embodiments, the mounting portion 810 may be resized, reshaped or omitted altogether. The mounting portion 810 is also configured to receive an inlet tube (note tube 184 in FIGS. 1-5) and, accordingly, has a recess 812, with a shoulder 814 that is configured to receive the inlet tube. A filter (not shown) may be positioned within the recess 812 between the inlet tube and the shoulder 814. The recess 812 and shoulder 814 may, alternatively, be associated with the fluid flow portion 806, or both the fluid flow portion and the mounting portion 810, in other implementations of the main check valve 800.

The exemplary valve element 804 (or “plunger”) includes a shaft 816 and a head 818. A spring retainer 820 is secured to the valve element 804. More specifically, the spring retainer 820 is secured to the end of the shaft 816 opposite the head 818 and may, for example, be press fit onto the shaft. The valve element 804 is biased to the closed position illustrated in FIG. 28 by a spring 824 (e.g. a coil spring) or other suitable biasing device. One end of the spring 824 abuts a shoulder 826 on the housing fluid flow portion 806, and the other end of the spring abuts the spring retainer 820.

An elastomeric valve seat 830 is positioned between the valve element head 818 and the end of the housing fluid flow portion 806. The valve seat 830 is sized and shaped such that it will be engaged by the valve element head 818, but not by the housing of the associated fluid transfer device, e.g. the fluid transfer device 100h. The valve seat 830 is an o-ring, which may be circular, semi-circular or otherwise curved in cross-section when in its uncompressed state, that has a generally annular shape and a central opening 833 through which fluid passes.

The fluid lumen 808 may be sized based on the desired fluid flow characteristics of the valve 800 and, in the illustrated embodiment is about 0.019 inch to about 0.035 inch in diameter. The valve seat opening 833 will typically be at least the same size as fluid lumen 808, and is larger in the illustrated embodiment, in order to prevent the valve seat 830 from interfering with fluid flow. Another factor associated with the size of the valve seat opening 833 is the size of the valve element shaft 816. More specifically, the opening 833 should be sized such that, in the event that the valve seat 830 is overcompressed and/or is off-center, the valve seat will be not impinge the valve element shaft 816. To that end, the exemplary valve element shaft 816 is about 0.014 inch to about 0.026 inch in diameter, and the exemplary valve seat opening 833 will be about 0.024 inch to about 0.045 inch in diameter.

An elastomeric gasket 844, which is a separate structural element from the valve seat 830, may be used to form a seal between the housing fluid flow portion 806 and the housing 102 or other structure into which the main check valve 800 is inserted. The outer diameter of the exemplary gasket 844 is slightly less (e.g. about 0.005 inch to about 0.020 inch in the exemplary embodiment) than the outer diameter of the housing fluid flow portion 806. This difference produces a gap 840 which allows the gasket 844 to deform (or “extrude”) outwardly when compressed in the manner illustrated in FIG. 29.

The exemplary main check valve 800 illustrated in FIG. 28 is provided with apparatus that controls the compression of the gasket 844. More specifically, the housing 802 includes a stop member 838 that extends from the end of the housing flow portion 806. The exemplary stop member 838 is a rigid, substantially annular structure that is integral with the housing flow portion 806, has an outer diameter that is less than that of the flow portion, and has a height H that is equal to, or is slightly less than (e.g. about 0.003 inch less than), the thickness of the gasket 844. The stop member 838, which is located outwardly of the valve seat 830, limits movement of the main check valve 800 relative to the structure into which the main check valve is inserted. More specifically, in the exemplary fluid transfer device 100g illustrated in FIG. 29, the stop member 838 engages the end wall 119 of the valve recess 118. It should also be noted that the stop member 838 may define a continuous perimeter (as shown) or, alternatively, be composed of a plurality of spaced elements that together define a discontinuous perimeter. The stop member 838, whether continuous or discontinuous, may also have an overall shape other than the illustrated substantially annular shape.

With respect to manufacturing and materials, the exemplary housing 802 is a machined part and suitable materials for the housing include, but are not limited to, titanium, titanium alloys, stainless steel (e.g. 316L stainless steel), cobalt-nickel alloys, and refractory metals such as tantalum. The valve element 804 may also be machined and suitable materials for the machined valve element include, but are not limited to, those described above in the context of the housing 802. Alternatively, as discussed in greater detail below, the valve element 804 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 830 and gasket 844 include, but are not limited to, elastomers such as silicone rubber, latex rubber, urethane, butyl rubber, and isoprene. The valve seat 830 will typically be separately manufactured structure that is positioned inwardly of the stop member 838. The gasket 844 may be molded directly onto the housing 802 by way of a co-molding (or “insert molding”) process. Here, the housing 802 is clamped into a mold that includes a cavity in the shape of the gasket 844, and the silicone rubber or other material is then injected into the cavity. A primer may be used to insure that the injected material sticks to the housing 802. Such co-molding is advantageous because it eliminates the tolerance stack-up that is associated with the assembly of separately manufactured parts. The gasket 844 may, alternatively, be separately manufactured, positioned around the stop member 838 and, if desired, secured to the housing 802 with adhesive.

There are a variety of advantages associated with the main check valve 800. For example, the main check valve 800 avoids the above-described valve seat over-compression problems by configuring the valve seat such that it does not engage the housing of the fluid transfer device into which the valve is inserted. More specifically, as illustrated in FIG. 29, there is no assembly-related compression of the valve seat 830 when the exemplary valve 800 is inserted into the fluid transfer device housing 102. The only force applied to the valve seat 830 is that associated with the spring 824. Moreover, even if there is some over-compression of the valve seat 830, the valve seat 830 will not impinge the valve element shaft 816 because the diameter of the opening 833 is relatively large.

Because the portion of the valve seat 830 that engages the valve element heat 818 is circular in cross-section, the contact area between valve seat and the valve element head 818 is decreased, which in turn increases the sealing pressure, as compared to a sealing arrangement that has two flat surfaces. Additionally, although the valve seat 830 will flatten slightly (FIG. 24) when the main check valve 800 is closed, thereby reducing the amount of flat on flat surface area as well as the adhesion force associated therewith, as compared to the main check valve illustrated in FIGS. 1-5. Compressing the valve seat raised seal 830 is also advantageous because silicone and other elastomeric materials tend to tear when in tension. The scuffing issue is also obviated because the sharp lateral edges of the valve element head 818 do not come into contact with the valve seat 830.

Turning to the gasket 844, compression of the gasket is a function of only two dimensions, i.e. the thickness of the gasket and the height H of the stop member 838, as compared to the three dimensions associated with the main check valve 107 illustrated in FIGS. 1-5. It is, therefore, substantially less likely that a tolerance stack-up will result in over-compression of the gasket 844, as compared to the valve seat in the main check valve 107 which also functions as a gasket. Compressing the gasket 844 is advantageous because silicone and other elastomeric materials tend to tear when in tension. Also, in those instances where the height of the gasket is greater than that of the stop member 838 increases sealing pressure in a manner that is relatively easy to manufacture. The gap 840 also allows the gasket 844 to deform outwardly when compressed. Additionally, even in those instances where the gasket 844 is over-compressed during assembly, the over-compression will not reduce the effectiveness of the valve seat 830 because the valve seat is not connected to the gasket.

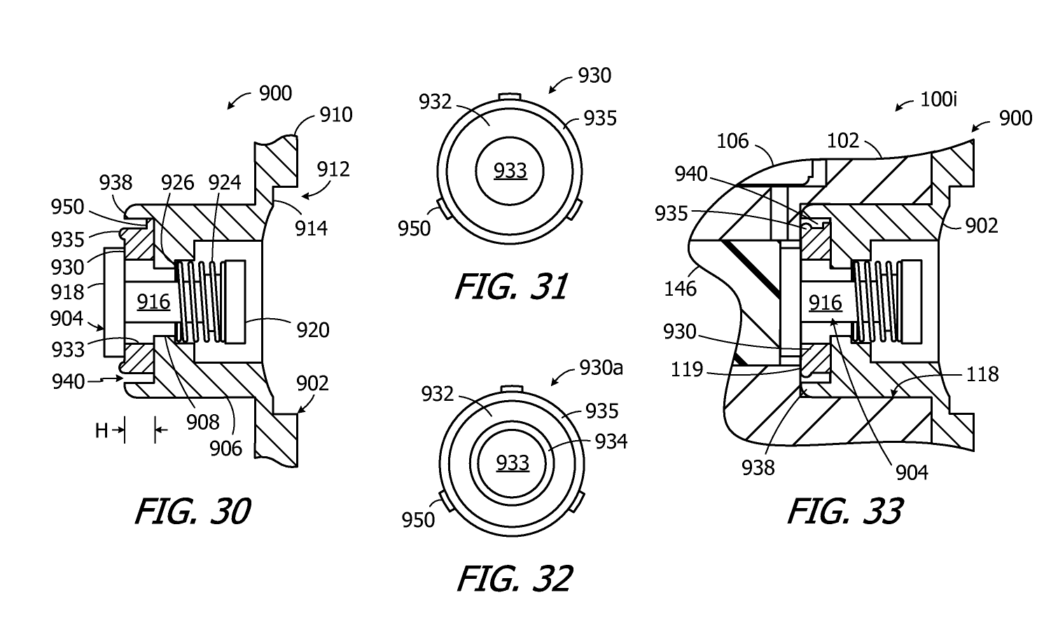

A main check valve in accordance with one embodiment of a present invention is generally represented by reference numeral 900 in FIG. 30. The exemplary main check valve 900 may be incorporated into a variety of fluid transfer devices. One such device is the fluid transfer device 100i illustrated in FIG. 33. The fluid transfer device 100i is essentially identical to the fluid transfer device 100, but for the substitution of main check valve 900 for main check valve 107, and similar elements are represented by similar reference numerals.

As illustrated for example in FIG. 30, the main check valve 900 includes a housing 902 and a valve element 904 that is movable relative to the housing 902. The exemplary housing 902 has a generally cylindrical fluid flow portion 906, with a fluid lumen 908 that is opened and closed by the valve element 904, and a mounting portion 910 that is used to secure the main check valve 900 to a fluid transfer device or other structure. In the illustrated embodiment, which is configured for use with a cylindrical fluid transfer device, the mounting portion 910 is disk-shaped. In other embodiments, the mounting portion 910 may be resized, reshaped or omitted altogether. The mounting portion 910 is also configured to receive an inlet tube (note tube 184 in FIGS. 1-5) and, accordingly, has a recess 912, with a shoulder 914 that is configured to receive the inlet tube. A filter (not shown) may be positioned within the recess 912 between the inlet tube and the shoulder 914. The recess 912 and shoulder 914 may, alternatively, be associated with the fluid flow portion 906, or both the fluid flow portion and the mounting portion 910, in other implementations of the main check valve 900.

The exemplary valve element 904 (or “plunger”) includes a shaft 916 and a head 918. A spring retainer 920 is secured to the valve element 904. More specifically, the spring retainer 920 is secured to the end of the shaft 916 opposite the head 918 and may, for example, be press fit onto the shaft. The valve element 904 is biased to the closed position illustrated in FIG. 20 by a spring 924 (e.g. a coil spring) or other suitable biasing device. One end of the spring 924 abuts a shoulder 926 on the housing fluid flow portion 906, and the other end of the spring abuts the spring retainer 920.

An elastomeric valve seat 930 is positioned between the valve element head 918 and the end of the housing fluid flow portion 906. The exemplary valve seat 930 has a base 932, with a central opening 933 through which fluid passes, an outer raised seal 935 that protrudes from the base. The exemplary base 932 is annularly shaped and is rectangular in cross-section, while exemplary raised seal 935 has a circular shape and is semi-circular or otherwise curved in cross-section. The raised seal 935 is aligned with the outer diameter of the base. Alternatively, the main check valve 900 may include the valve seat 930a illustrated in FIG. 32, which also has an inner raised seal 934 that is also semi-circular or otherwise curved in cross-section and protrudes from the base 932. The valve element head 918 will engage and compress the inner raised seal 934 when the valve is closed.

The fluid lumen 908 may be sized based on the desired fluid flow characteristics of the valve 900 and, in the illustrated embodiment is about 0.019 inch to about 0.035 inch in diameter. The valve seat opening 933 will (in both instances) typically be at least the same size as fluid lumen 908, and is larger in the illustrated embodiment, in order to prevent the valve seat 930 from interfering with fluid flow. Another factor associated with the size of the valve seat opening 933 is the size of the valve element shaft 916. More specifically, the opening 933 should be sized such that, in the event that the valve seat 930 is overcompressed and/or is off-center, the valve seat will be not impinge the valve element shaft 916. To that end, the exemplary valve element shaft 916 is about 0.014 inch to about 0.026 inch in diameter, and the exemplary valve seat opening 933 will be about 0.024 inch to about 0.045 inch in diameter.

Valve seats 930 and 930a are both provided with alignment tabs 950 that perform the function of centering the valve seats generally, and the opening 933 in particular, relative to the fluid lumen 908 and valve element shaft 916. So centered, the fluid lumen 908, valve element shaft 916 and opening 933 define a common longitudinal axis (i.e. are coaxial). The alignment tabs 950 extend radially outward from the base 932 and are sized such that they define an outer diameter that is slightly greater than (for an interference fit), or is equal to, the inner diameter of the stop member 938 (discussed below). Additionally, although three (3) alignment tabs are provided in the illustrated embodiments, the number of tabs may be increased or decreased as desired.

The exemplary main check valve 900 illustrated in FIG. 30 is provided with apparatus that prevents over-compression of the elastomeric valve seat 930. More specifically, the housing 902 includes a stop member 938 that extends from the end of flow portion 906. The exemplary stop member 938 is a rigid, substantially annular structure that is integral with the flow portion 906 and has a height H that is equal to, or is slightly less than (e.g. about 0.003 inch less than), the thickness of the elastomeric valve seat 930. The stop member 938, which is located outwardly of the valve seat 930, limits movement of the main check valve 900 relative to the structure into which the main check valve is inserted. More specifically, in the exemplary fluid transfer device 100i illustrated in FIG. 33, the stop member 938 engages the end wall 119 of the valve recess 118. The inner diameter of the stop member 938 is slightly greater (e.g. about 0.005 inch to about 0.020 inch) than the outer diameter of the valve seat base 932. This results in a gap 940 into which the valve seat 930 can deform (or “extrude”) when compressed in the manner illustrated in FIG. 33. It should also be noted that the stop member 938 may define a continuous perimeter (as shown) or, alternatively, be composed of a plurality of spaced elements that together define a discontinuous perimeter. The stop member 938, whether continuous or discontinuous, may also have an overall shape other than the illustrated substantially annular shape.

With respect to manufacturing and materials, the exemplary housing 902 is a machined part and suitable materials for the housing include, but are not limited to, titanium, titanium alloys, stainless steel (e.g. 316L stainless steel), cobalt-nickel alloys, and refractory metals such as tantalum. The valve element 904 may also be machined and suitable materials for the machined valve element include, but are not limited to, those described above in the context of the housing 902. Alternatively, as discussed in greater detail below, the valve element 904 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seats 930 and 930a include, but are not limited to, elastomers such as silicone rubber, latex rubber, urethane, butyl rubber, and isoprene. The valve seats 930 and 930a may be molded and positioned on a housing 902 in the manner illustrated in FIG. 30. As noted above, the alignment tabs 950 center the valve seats relative to the fluid lumen 908 and valve element shaft 916. If desired, adhesive may be used to secure the valve seat 930 (or 930a) to the housing 902.

There are a variety of advantages associated with the main check valve 900. For example, compression of the valve seat 930 (or 930a) in the exemplary main check valve 900 is a function of only two dimensions, i.e. the thickness of the valve seat and the height H of the stop member 938, as compared to the three dimensions associated with the main check valve 107 illustrated in FIGS. 1-5. It is, therefore, substantially less likely that a tolerance stack-up will result in over-compression of the valve seat 930, as compared to the main check valve 107. Additionally, even if there is some over-compression of the valve seat 930 (or 930a), the valve seat 930 will not impinge the valve element shaft 916 because the diameter of the opening 933 is relatively large. The likelihood of shaft impingement is further reduced by the fact that the alignment tabs 950 center the valve seat 930 (or 930a), particularly the opening 933, relative to the valve element shaft 916. The gap 940 will also allow the valve seat 930 (or 930a) to deform outwardly, thereby preventing the formation of an irregular sealing surface.

Turning to the outer raised seal 935 of the valve seat 930 (or 930a), the outer raised seal will be under compression when the main check valve 900 is inserted into the valve recess 118 of the housing 102. Compressing the raised seal 935 is advantageous because silicone and other elastomeric materials tend to tear when in tension. The raised seal 935 also increases sealing pressure without increasing the size of entire valve seat and does so in a manner that is relatively easy to manufacture.

The inner raised seal 934 in the valve seat 930a reduces the contact area between valve seat and the valve element head 918, which in turn increases the sealing pressure, as compared to a sealing arrangement that has two flat surfaces. Additionally, although the raised seal 934 will flatten slightly when the main check valve 900 is closed, the curved raised seal will reduce the amount of flat on flat surface area as well as the adhesion force associated therewith, as compared to the main check valve illustrated in FIGS. 1-5. Compressing the valve seat raised seal 934 is also advantageous because silicone and other elastomeric materials tend to tear when in tension. The scuffing issue is also obviated because the sharp lateral edges of the valve element head 918 will not come into contact with the valve seat 930.

Turning to the alignment tabs 950, the alignment tabs insure that the opening 933 is centered with respect to the valve element head 918 which, in turn, insures that the valve element head will be properly aligned with the valve seat 930 (or 930a) when the valve 900 is closed.

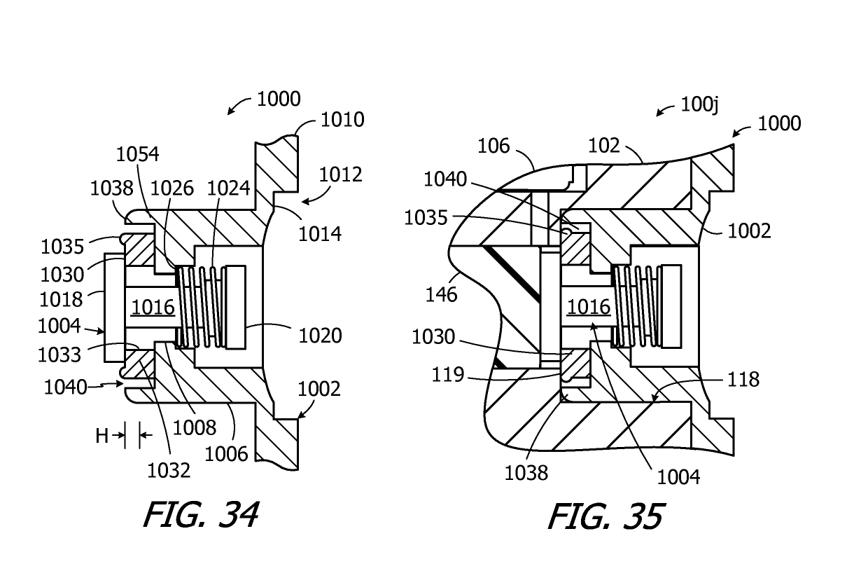

A main check valve in accordance with one embodiment of a present invention is generally represented by reference numeral 1000 in FIG. 34. The exemplary main check valve 1000 may be incorporated into a variety of fluid transfer devices. One such device is the fluid transfer device 100j illustrated in FIG. 35. The fluid transfer device 100j is essentially identical to the fluid transfer device 100, but for the substitution of main check valve 1000 for main check valve 107, and similar elements are represented by similar reference numerals.

As illustrated for example in FIG. 34, the main check valve 1000 includes a housing 1002 and a valve element 1004 that is movable relative to the housing 1002. The exemplary housing 1002 has a generally cylindrical fluid flow portion 1006, with a fluid lumen 1008 that is opened and closed by the valve element 1004, and a mounting portion 1010 that is used to secure the main check valve 1000 to a fluid transfer device or other structure. In the illustrated embodiment, which is configured for use with a cylindrical fluid transfer device, the mounting portion 1010 is disk-shaped. In other embodiments, the mounting portion 1010 may be resized, reshaped or omitted altogether. The mounting portion 1010 is also configured to receive an inlet tube (note tube 184 in FIGS. 1-5) and, accordingly, has a recess 1012, with a shoulder 1014 that is configured to receive the inlet tube. A filter (not shown) may be positioned within the recess 1012 between the inlet tube and the shoulder 1014. The recess 1012 and shoulder 1014 may, alternatively, be associated with the fluid flow portion 1006, or both the fluid flow portion and the mounting portion 1010, in other implementations of the main check valve 1000.

The exemplary valve element 1004 (or “plunger”) includes a shaft 1016 and a head 1018. A spring retainer 1020 is secured to the valve element 1004. More specifically, the spring retainer 1020 is secured to the end of the shaft 1016 opposite the head 1018 and may, for example, be press fit onto the shaft. The valve element 1004 is biased to the closed position illustrated in FIG. 20 by a spring 1024 (e.g. a coil spring) or other suitable biasing device. One end of the spring 1024 abuts a shoulder 1026 on the housing fluid flow portion 1006, and the other end of the spring abuts the spring retainer 1020.

An elastomeric valve seat 1030 is positioned between the valve element head 1018 and the end of the housing fluid flow portion 1006. The exemplary valve seat 1030 includes a base 1032, which has a central opening 1033 through which fluid passes, and a raised seal 1035 that protrudes from the base. The exemplary base 1032 is annularly shaped and is rectangular in cross-section, while the exemplary raised seal 1035 has a circular shape and is semi-circular or otherwise curved in cross-section. The raised seal 1035 extends along the outer diameter of the base 1032, and the inner diameter of the raised seal is larger than the outer diameter of the valve element head 1018. Additionally, in other implementations, the elastomeric valve seat may be provided with an inner raised seal similar to the inner raised seal 934 of the valve seat 930a (FIG. 32).

The fluid lumen 1008 may be sized based on the desired fluid flow characteristics of the valve 1000 and, in the illustrated embodiment is about 0.019 inch to about 0.035 inch in diameter. The valve seat opening 1033 will typically be at least the same size as fluid lumen 1008, and is larger in the illustrated embodiment, in order to prevent the valve seat 1030 from interfering with fluid flow. Another factor associated with the size of the valve seat opening 1033 is the size of the valve element shaft 1016. More specifically, the opening 1033 should be sized such that, in the event that the valve seat 1030 is overcompressed and/or is off-center, the valve seat will be not impinge the valve element shaft 1016. To that end, the exemplary valve element shaft 1016 is about 0.014 inch to about 0.026 inch in diameter, and the exemplary valve seat opening 1033 will be about 0.024 inch to about 0.045 inch in diameter.

The valve seat 1030 may be molded or co-molded into the housing 1002. Co-molding insures that the valve seat 1030 will be centered relative to the fluid lumen 1008 and valve element shaft 1016. So centered, the fluid lumen 1008, valve element shaft 1016 and opening 1033 define a common longitudinal axis (i.e. are coaxial). This, in turn, insures that the valve element head 1018 will be properly aligned with the valve seat 1030 when the valve 1000 is closed.

The exemplary main check valve 1000 illustrated in FIG. 34 is provided with apparatus that prevents over-compression of the elastomeric valve seat 1030. More specifically, the housing 1002 includes a stop member 1038 that extends from the end of flow portion 1006. The exemplary stop member 1038 is a rigid, substantially annular structure that is integral with the flow portion 1006 and has a height H that is equal to, or is slightly less than (e.g. about 0.003 inch less than), the thickness of the elastomeric valve seat 1030. The stop member 1038, which is located outwardly of the valve seat 1030, limits movement of the main check valve 1000 relative to the structure into which the main check valve is inserted. More specifically, in the exemplary fluid transfer device 100j illustrated in FIG. 35, the stop member 1038 engages the end wall 119 of the valve recess 118. The inner diameter of the stop member 1038 is slightly greater (e.g. about 0.005 inch to about 0.020 inch) than the outer diameter of the valve seat base 1032. This results in a gap 1040 into which the valve seat 1030 can deform (or “extrude”) when compressed in the manner illustrated in FIG. 35. It should also be noted that the stop member 1038 may define a continuous perimeter (as shown) or, alternatively, be composed of a plurality of spaced elements that together define a discontinuous perimeter. The stop member 1038, whether continuous or discontinuous, may also have an overall shape other than the illustrated substantially annular shape.

With respect to manufacturing and materials, the exemplary housing 1002 is a machined part and suitable materials for the housing include, but are not limited to, titanium, titanium alloys, stainless steel (e.g. 316L stainless steel), cobalt-nickel alloys, and refractory metals such as tantalum. The valve element 1004 may also be machined and suitable materials for the machined valve element include, but are not limited to, those described above in the context of the housing 1002. Alternatively, as discussed in greater detail below, the valve element 1004 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1030 include, but are not limited to, elastomers such as silicone rubber, latex rubber, urethane, butyl rubber, and isoprene.

As noted above, valve seat 1030 may be molded directly onto the housing 1002 by way of a co-molding (or “insert molding”) process. Here, the housing 1002 is clamped into a mold that includes a cavity in the shape of the valve seat 1030, and the silicone rubber or other material is then injected into the cavity. A primer may be used to insure that the injected material sticks to the housing 1002. Such co-molding is advantageous because it eliminates the tolerance stack-up that is associated with the assembly of separately manufactured parts.

There are a variety of advantages associated with the main check valve 1000. For example, compression of the valve seat 1030 in the exemplary main check valve 1000 is a function of only two dimensions, i.e. the thickness of the valve seat and the height H of the stop member 1038, as compared to the three dimensions associated with the main check valve 107 illustrated in FIGS. 1-5. The molding (or co-molding) further reduces tolerance stack-up. It is, therefore, substantially less likely that a tolerance stack-up will result in over-compression of the valve seat 1030, as compared to the main check valve 107. Additionally, even if there is some over-compression of the valve seat 1030, the valve seat 1030 will not impinge the valve element shaft 1016 because the diameter of the opening 1033 is relatively large. The likelihood of shaft impingement is further reduced by the fact that the alignment ring 1052 centers the valve seat 1030, particularly the opening 1033, relative to the valve element shaft 1016. The gap 1040 will also allow the valve seat 1030 to deform outwardly, thereby preventing the formation of an irregular sealing surface.

Turning to the outer raised seal 1035 of the valve seat 1030, the outer raised seal will be under compression when the main check valve 1000 is inserted into the valve recess 118 of the housing 102. Compressing the raised seal 1035 is advantageous because silicone and other elastomeric materials tend to tear when in tension. The raised seal 1035 also increases sealing pressure without increasing the size of entire valve seat and does so in a manner that is relatively easy to manufacture.

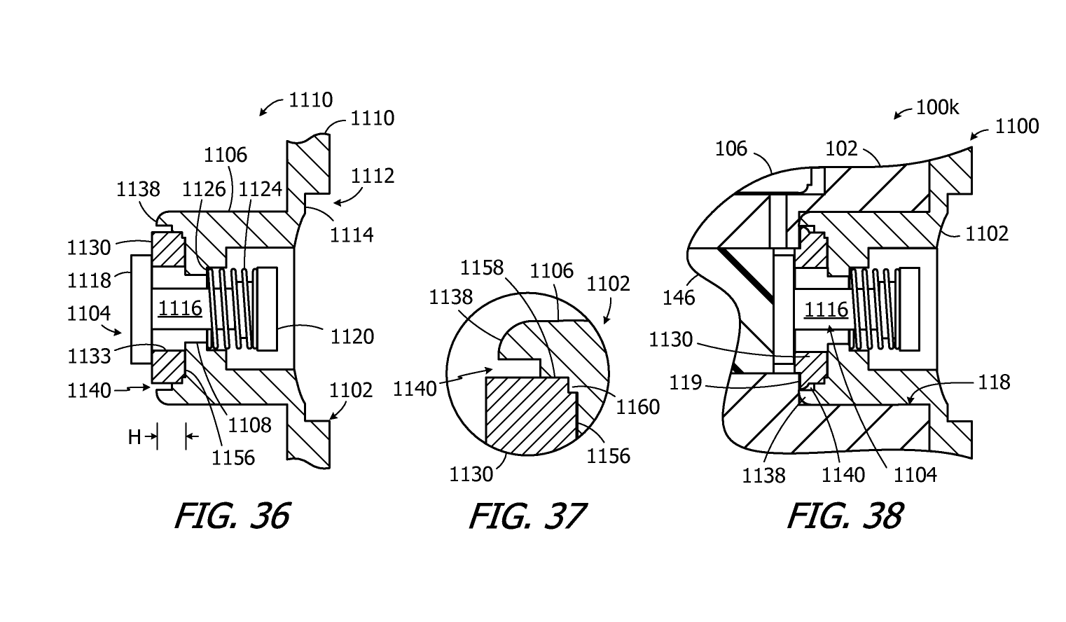

A main check valve in accordance with one embodiment of a present invention is generally represented by reference numeral 1100 in FIG. 36. The exemplary main check valve 1100 may be incorporated into a variety of fluid transfer devices. One such device is the fluid transfer device 100k illustrated in FIG. 38. The fluid transfer device 100k is essentially identical to the fluid transfer device 100, but for the substitution of main check valve 1100 for main check valve 107, and similar elements are represented by similar reference numerals.

As illustrated for example in FIG. 36, the main check valve 1100 includes a housing 1102 and a valve element 1104 that is movable relative to the housing 1102. The exemplary housing 1102 has a generally cylindrical fluid flow portion 1106, with a fluid lumen 1108 that is opened and closed by the valve element 1104, and a mounting portion 1110 that is used to secure the main check valve 1100 to a fluid transfer device or other structure. In the illustrated embodiment, which is configured for use with a cylindrical fluid transfer device, the mounting portion 1110 is disk-shaped. In other embodiments, the mounting portion 1110 may be resized, reshaped or omitted altogether. The mounting portion 1110 is also configured to receive an inlet tube (note tube 184 in FIGS. 1-5) and, accordingly, has a recess 1112, with a shoulder 1114 that is configured to receive the inlet tube. A filter (not shown) may be positioned within the recess 1112 between the inlet tube and the shoulder 1114. The recess 1112 and shoulder 1114 may, alternatively, be associated with the fluid flow portion 1106, or both the fluid flow portion and the mounting portion 1110, in other implementations of the main check valve 1100.

The exemplary valve element 1104 (or “plunger”) includes a shaft 1116 and a head 1118. A spring retainer 1120 is secured to the valve element 1104. More specifically, the spring retainer 1120 is secured to the end of the shaft 1116 opposite the head 1118 and may, for example, be press fit onto the shaft. The valve element 1104 is biased to the closed position illustrated in FIG. 36 by a spring 1124 (e.g. a coil spring) or other suitable biasing device. One end of the spring 1124 abuts a shoulder 1126 on the housing fluid flow portion 1106, and the other end of the spring abuts the spring retainer 1120.

An elastomeric valve seat 1130 is positioned between the valve element head 1118 and a valve seat support surface 1156 at the end of the housing fluid flow portion 1106. The exemplary valve seat 1130 has a generally annular shape and a central opening 1133 through which fluid passes.

The fluid lumen 1108 may be sized based on the desired fluid flow characteristics of the valve 1100 and, in the illustrated embodiment is about 0.019 inch to about 0.035 inch in diameter. The valve seat opening 1133 will typically be at least the same size as fluid lumen 1108, and is larger in the illustrated embodiment, in order to prevent the valve seat 1130 from interfering with fluid flow. Another factor associated with the size of the valve seat opening 1133 is the size of the valve element shaft 1116. More specifically, the opening 1133 should be sized such that, in the event that the valve seat 1130 is overcompressed and/or is off-center, the valve seat will be not impinge the valve element shaft 1116. To that end, the exemplary valve element shaft 1116 is about 0.014 inch to about 0.026 inch in diameter, and the exemplary valve seat opening 1133 will be about 0.024 inch to about 0.045 inch in diameter.

The exemplary main check valve 1100 illustrated in FIG. 36 is provided with apparatus that prevents over-compression of the elastomeric valve seat 1130. More specifically, the housing 1102 includes a stop member 1138 that extends from the end of flow portion 1106. The exemplary stop member 1138 is a rigid, substantially annular structure that is integral with the flow portion 1106 and has a height H that is equal to, or is slightly less than (e.g. about 0.003 inch less than), the thickness of the elastomeric valve seat 1130. The stop member 1138, which is located outwardly of the valve seat 1130, limits movement of the main check valve 1100 relative to the structure into which the main check valve is inserted. More specifically, in the exemplary fluid transfer device 100k illustrated in FIG. 38, the stop member 1138 engages the end wall 119 of the valve recess 118. It should also be noted that the stop member 1138 may define a continuous perimeter (as shown) or, alternatively, be composed of a plurality of spaced elements that together define a discontinuous perimeter. The stop member 1138, whether continuous or discontinuous, may also have an overall shape other than the illustrated substantially annular shape.

The outer diameter of valve seat 1130 is slightly less (e.g. about 0.005 inch to about 0.020 inch in the exemplary embodiment) than the inner diameter of the stop member 1138. This difference produces a gap 1140 that allows the valve seat 1130 to deform (or “extrude”) outwardly when compressed in the manner illustrated in FIG. 38.

Turning to FIG. 37, the exemplary housing 1102 includes an alignment surface 1158 which performs the function of centering the valve seat 1130 generally, and the opening 1133 in particular, relative to the fluid lumen 1108 and valve element shaft 1116. So centered, the fluid lumen 1008, valve element shaft 1016 and opening 1033 define a common longitudinal axis (i.e. are coaxial). The alignment surface 1158 is, in the illustrated embodiment, an inwardly facing continuous cylindrical surface that is centered relative to relative to the fluid lumen 1108 and valve element shaft 1116. The alignment surface 1158 also has an inner diameter that is substantially equal to the outer diameter of the valve seat 1130. As such, the alignment surface 1158 holds the valve seat 1130 in the centered position illustrated in FIG. 36.

It should be noted here that other types of alignment surfaces may be employed. By way of example, but not limitation, the housing 1102 could alternatively include a plurality of spaced alignment members that extend inwardly from the stop member 1138, engage the valve seat 1130, and define a discontinuous alignment surface.

The exemplary housing 102 also includes an annular protrusion 1160 that extends away from the housing valve seat support surface 1156 and inwardly from the alignment surface 1158. The annular protrusion 1160 performs the function of creating a stress riser in the valve seat 1130 that is aligned with the end wall 119 of the housing valve recess 118.

With respect to manufacturing and materials, the exemplary housing 1102 is a machined part and suitable materials for the housing include, but are not limited to, titanium, titanium alloys, stainless steel (e.g. 316L stainless steel), cobalt-nickel alloys, and refractory metals such as tantalum. The valve element 1104 may also be machined and suitable materials for the machined valve element include, but are not limited to, those described above in the context of the housing 1102. Alternatively, as discussed in greater detail below, the valve element 1104 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1130 include, but are not limited to, elastomers such as silicone rubber, latex rubber, urethane, butyl rubber, and isoprene. The valve seat 1130 may be molded and positioned on the housing 1102, and against the alignment surface 1158 and annular protrusion 1160, in the manner illustrated in FIG. 36. As noted above, the alignment surface 1158 centers the valve seat 1130 relative to the fluid lumen 1108 and valve element shaft 1116, while the annular protrusion 1160 creates a stress riser. If desired, adhesive may be used to secure the valve seat 1130 to the housing 1102.

There are a variety of advantages associated with the main check valve 1100. For example, compression of the valve seat 1130 in the exemplary main check valve 1100 is a function of only two dimensions, i.e. the thickness of the valve seat and the height H of the stop member 1138, as compared to the three dimensions associated with the main check valve 107 illustrated in FIGS. 1-5. It is, therefore, substantially less likely that a tolerance stack-up will result in over-compression of the valve seat 1130, as compared to the main check valve 107. Additionally, even if there is some over-compression of the valve seat 1130, the valve seat will not impinge the valve element shaft 1116 because the diameter of the opening 1133 is relatively large, while the gap 1140 will allow the valve seat to deform outwardly, thereby preventing the formation of an irregular sealing surface. The likelihood of shaft impingement is further reduced by the fact that the alignment surface 1158 centers the valve seat 1130, particularly the opening 1133, relative to the valve element shaft 1116. Turning to the annular protrusion 1160, the stress riser in the valve seat 1130 created by the annular protrusion increases the compression of the portion of the valve seat 1130 that engages, and forms a seal with, the fluid transfer device housing 102 without distorting the portion of the valve seat that is engaged by the valve element head 1118. Compressing the valve seat is also advantageous because silicone and other elastomeric materials tend to tear when in tension.

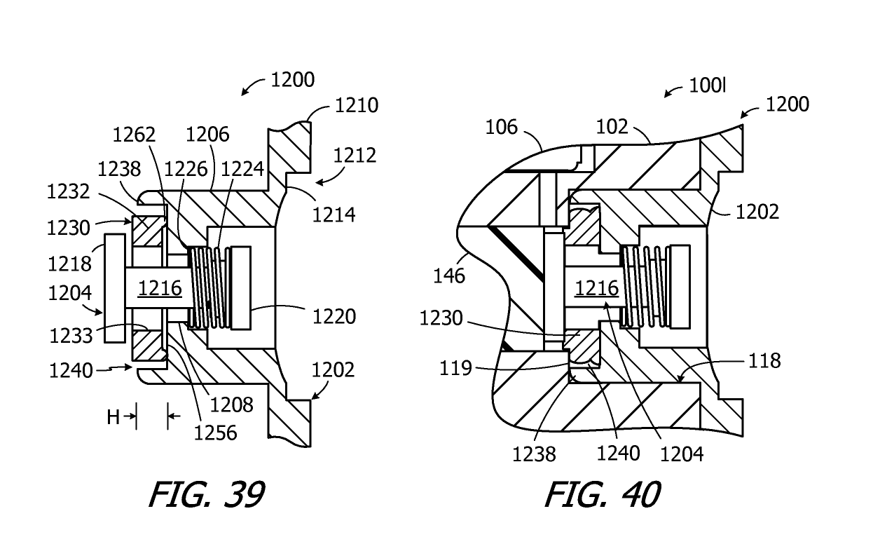

A main check valve in accordance with one embodiment of a present invention is generally represented by reference numeral 1200 in FIG. 39. The exemplary main check valve 1200 may be incorporated into a variety of fluid transfer devices. One such device is the fluid transfer device 100l illustrated in FIG. 40. The fluid transfer device 100l is essentially identical to the fluid transfer device 100, but for the substitution of main check valve 1200 for main check valve 107, and similar elements are represented by similar reference numerals.

As illustrated for example in FIG. 39, the main check valve 1200 includes a housing 1202 and a valve element 1204 that is movable relative to the housing 1202. The exemplary housing 1202 has a generally cylindrical fluid flow portion 1206, with a fluid lumen 1208 that is opened and closed by the valve element 1204, and a mounting portion 1210 that is used to secure the main check valve 1200 to a fluid transfer device or other structure. In the illustrated embodiment, which is configured for use with a cylindrical fluid transfer device, the mounting portion 1210 is disk-shaped. In other embodiments, the mounting portion 1210 may be resized, reshaped or omitted altogether. The mounting portion 1210 is also configured to receive an inlet tube (note tube 184 in FIGS. 1-5) and, accordingly, has a recess 1212, with a shoulder 1214 that is configured to receive the inlet tube. A filter (not shown) may be positioned within the recess 1212 between the inlet tube and the shoulder 1214. The recess 1212 and shoulder 1214 may, alternatively, be associated with the fluid flow portion 1206, or both the fluid flow portion and the mounting portion 1210, in other implementations of the main check valve 1200.

The exemplary valve element 1204 (or “plunger”) includes a shaft 1216 and a head 1218. A spring retainer 1220 is secured to the valve element 1204. More specifically, the spring retainer 1220 is secured to the end of the shaft 1216 opposite the head 1218 and may, for example, be press fit onto the shaft. The valve element 1204 is biased to the closed position illustrated in FIG. 40 by a spring 1224 (e.g. a coil spring) or other suitable biasing device. One end of the spring 1224 abuts a shoulder 1226 on the housing fluid flow portion 1206, and the other end of the spring abuts the spring retainer 1220.

An elastomeric valve seat 1230 is positioned between the valve element head 1218 and a valve seat support surface 1256 at the end of the housing fluid flow portion 1206. The exemplary valve seat 1230 has a base 1232, with a central opening 1233 through which fluid passes, and a protrusion 1262 that extends from the base and abuts the support surface 1256. The protrusion 1262 performs the function of creating a stress riser in the valve seat 1230 that is aligned with the end wall 119 of the housing valve recess 118. The exemplary base 1232 is annularly shaped and is rectangular in cross-section, while the exemplary protrusion 1262 has a circular shape and is semi-circular or otherwise curved in cross-section. The protrusion 1262 extends along the outer diameter of the base 1232 and may, alternatively, have other shapes such as an annular shape that is rectangular in cross-section.

The fluid lumen 1208 may be sized based on the desired fluid flow characteristics of the valve 1200 and, in the illustrated embodiment is about 0.019 inch to about 0.035 inch in diameter. The valve seat opening 1233 will typically be at least the same size as fluid lumen 1208, and is larger in the illustrated embodiment, in order to prevent the valve seat 1230 from interfering with fluid flow. Another factor associated with the size of the valve seat opening 1233 is the size of the valve element shaft 1216. More specifically, the opening 1233 should be sized such that, in the event that the valve seat 1230 is overcompressed and/or is off-center, the valve seat will be not impinge the valve element shaft 1216. To that end, the exemplary valve element shaft 1216 is about 0.014 inch to about 0.026 inch in diameter, and the exemplary valve seat opening 1233 will be about 0.024 inch to about 0.045 inch in diameter.

The exemplary main check valve 1200 illustrated in FIG. 39 is also provided with apparatus that prevents over-compression of the elastomeric valve seat 1230. More specifically, the housing 1202 includes a stop member 1238 that extends from the end of flow portion 1206. The exemplary stop member 1238 is a rigid, substantially annular structure that is integral with the flow portion 1206 and has a height H that is equal to, or is slightly less than (e.g. about 0.003 inch less than), the thickness of the elastomeric valve seat 1230. The stop member 1238, which is located outwardly of the valve seat 1230, limits movement of the main check valve 1200 relative to the structure into which the main check valve is inserted. More specifically, in the exemplary fluid transfer device 100l illustrated in FIG. 40, the stop member 1238 engages the end wall 119 of the valve recess 118. It should also be noted that the stop member 1238 may define a continuous perimeter (as shown) or, alternatively, be composed of a plurality of spaced elements that together define a discontinuous perimeter. The stop member 1238, whether continuous or discontinuous, may also have an overall shape other than the illustrated substantially annular shape.

It should also be noted that the outer diameter of valve seat 1230 is slightly less (e.g. about 0.005 inch to about 0.020 inch in the exemplary embodiment) than the inner diameter of the stop member 1238. This difference produces a gap 1240 that allows the valve seat 1230 to deform (or “extrude”) outwardly when compressed in the manner illustrated in FIG. 40.

With respect to manufacturing and materials, the exemplary housing 1202 is a machined part and suitable materials for the housing include, but are not limited to, titanium, titanium alloys, stainless steel (e.g. 316L stainless steel), cobalt-nickel alloys, and refractory metals such as tantalum. The valve element 1204 may also be machined and suitable materials for the machined valve element include, but are not limited to, those described above in the context of the housing 1202. Alternatively, as discussed in greater detail below, the valve element 1204 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1230 include, but are not limited to, elastomers such as silicone rubber, latex rubber, urethane, butyl rubber, and isoprene. The valve seat 1230 may be molded and positioned on a housing 1202 in the manner illustrated in FIG. 39. If desired, adhesive may be used to secure the valve seat 1230 to the housing 1202.

There are a variety of advantages associated with the main check valve 1200. For example, compression of the valve seat 1230 in the exemplary main check valve 1200 is a function of only two dimensions, i.e. the thickness of the valve seat and the height H of the stop member 1238, as compared to the three dimensions associated with the main check valve 107 illustrated in FIGS. 1-5. It is, therefore, substantially less likely that a tolerance stack-up will result in over-compression of the valve seat 1230, as compared to the main check valve 107. Additionally, even if there is some over-compression of the valve seat 1230, the valve seat will not impinge the valve element shaft 1216 because the diameter of the opening 1233 is relatively large, while the gap 1240 will allow the valve seat to deform outwardly, thereby preventing the formation of an irregular sealing surface. Turning to the protrusion 1262, the stress riser in the valve seat 1230 created by the protrusion increases the compression of the portion of the valve seat 1230 that engages, and forms a seal with, the fluid transfer device housing 102 without distorting the portion of the valve seat that is engaged by the valve element head 1218. Compressing the valve seat protrusion 1262 is also advantageous because silicone and other elastomeric materials tend to tear when in tension.

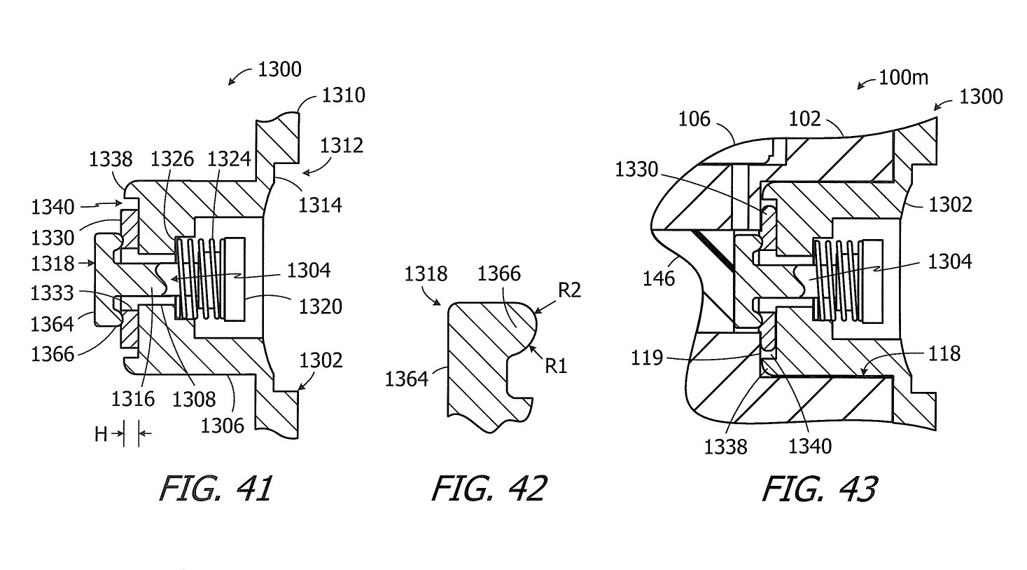

A main check valve in accordance with one embodiment of a present invention is generally represented by reference numeral 1300 in FIG. 41. The exemplary main check valve 1300 may be incorporated into a variety of fluid transfer devices. One such device is the fluid transfer device 100m illustrated in FIG. 43. The fluid transfer device 100m is essentially identical to the fluid transfer device 100, but for the substitution of main check valve 1300 for main check valve 107, and similar elements are represented by similar reference numerals.

As illustrated for example in FIG. 41, the main check valve 1300 includes a housing 1302 and a valve element 1304 that is movable relative to the housing 1302. The exemplary housing 1302 has a generally cylindrical fluid flow portion 1306, with a fluid lumen 1308 that is opened and closed by the valve element 1304, and a mounting portion 1310 that is used to secure the main check valve 1300 to a fluid transfer device or other structure. In the illustrated embodiment, which is configured for use with a cylindrical fluid transfer device, the mounting portion 1310 is disk-shaped. In other embodiments, the mounting portion 1310 may be resized, reshaped or omitted altogether. The mounting portion 1310 is also configured to receive an inlet tube (note tube 184 in FIGS. 1-5) and, accordingly, has a recess 1312, with a shoulder 1314 that is configured to receive the inlet tube. A filter (not shown) may be positioned within the recess 1312 between the inlet tube and the shoulder 1314. The recess 1312 and shoulder 1314 may, alternatively, be associated with the fluid flow portion 1306, or both the fluid flow portion and the mounting portion 1310, in other implementations of the main check valve 1300.

The exemplary valve element 1304 (or “plunger”) includes a shaft 1316 and a head 1318. A spring retainer 1320 is secured to the valve element 1304. More specifically, the spring retainer 1320 is secured to the end of the shaft 1316 opposite the head 1318 and may, for example, be press fit onto the shaft. The head 1318 has a main portion 1364 and a curved seal 1366 that is semi-circular or otherwise curved in cross-section and protrudes from the main portion. The seal 1366 is rigid and engages the elastomeric valve seat 1330 (discussed below) when the valve 1300 is closed. Additionally, in the illustrated embodiment, the curved seal 1366 has an overall circular shape that engages a circular portion of the valve seat 1330 as well as the complex radius cross-sectional shape illustrated in FIG. 42, where R1 is about 40% greater than R2.

The valve element 1304 is biased to the closed position illustrated in FIG. 41 by a spring 1324 (e.g. a coil spring) or other suitable biasing device. One end of the spring 1324 abuts a shoulder 1326 on the housing fluid flow portion 1306, and the other end of the spring abuts the spring retainer 1320.

An elastomeric valve seat 1330 is positioned between the valve element head 1318 and the end of the housing fluid flow portion 1306. The exemplary valve seat 1330 has a generally annular shape, a central opening 1333 through which fluid passes, and a sealing surface (i.e. the surface engaged by the valve element 1304) that is flat.

The fluid lumen 1308 may be sized based on the desired fluid flow characteristics of the valve 1300 and, in the illustrated embodiment is about 0.019 inch to about 0.035 inch in diameter. The valve seat opening 1333 will typically be at least the same size as fluid lumen 1308, and is larger in the illustrated embodiment, in order to prevent the valve seat 1330 from interfering with fluid flow. Another factor associated with the size of the valve seat opening 1333 is the size of the valve element shaft 1316. More specifically, the opening 1333 should be sized such that, in the event that the valve seat 1330 is overcompressed and/or is off-center, the valve seat will be not impinge the valve element shaft 1316. To that end, the exemplary valve element shaft 1316 is about 0.014 inch to about 0.026 inch in diameter, and the exemplary valve seat opening 1333 will be about 0.024 inch to about 0.045 inch in diameter.

The exemplary main check valve 1300 illustrated in FIG. 41 is also provided with apparatus that prevents over-compression of the elastomeric valve seat 1330. More specifically, the housing 1302 includes a stop member 1338 that extends from the end of flow portion 1306. The exemplary stop member 1338 is a rigid, substantially annular structure that is integral with the flow portion 1306 and has a height H that is equal to, or is slightly less than (e.g. about 0.003 inch less than), the thickness of the elastomeric valve seat 1330. The stop member 1338, which is located outwardly of the valve seat 1330, limits movement of the main check valve 1300 relative to the structure into which the main check valve is inserted. More specifically, in the exemplary fluid transfer device 100m illustrated in FIG. 43, the stop member 1338 engages the end wall 119 of the valve recess 118. It should also be noted that the stop member 1338 may define a continuous perimeter (as shown) or, alternatively, be composed of a plurality of spaced elements that together define a discontinuous perimeter. The stop member 1338, whether continuous or discontinuous, may also have an overall shape other than the illustrated substantially annular shape.

The outer diameter of valve seat 1330 is slightly less (e.g. about 0.005 inch to about 0.020 inch in the exemplary embodiment) than the inner diameter of the stop member 1338. This difference produces a gap 1340 that allows the valve seat 1330 to deform (or “extrude”) outwardly when compressed in the manner illustrated in FIG. 43.

With respect to manufacturing and materials, the exemplary housing 1302 is a machined part and suitable materials for the housing include, but are not limited to, titanium, titanium alloys, stainless steel (e.g. 316L stainless steel), cobalt-nickel alloys, and refractory metals such as tantalum. The valve element 1304 may also be machined and suitable materials for the machined valve element include, but are not limited to, those described above in the context of the housing 1302. Alternatively, as discussed in greater detail below, the valve element 1304 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1330 include, but are not limited to, elastomers such as silicone rubber, latex rubber, urethane, butyl rubber, and isoprene. The valve seat 1330 may be molded directly onto the housing 1302 by way of a co-molding (or “insert molding”) process. Here, the housing 1302 is clamped into a mold that includes a cavity in the shape of the valve seat 1330, and the silicone rubber or other material is then injected into the cavity. A primer may be used to insure that the injected material sticks to the housing 1302. Such co-molding is advantageous because it eliminates the tolerance stack-up that is associated with the assembly of separately manufactured parts. Nevertheless, the valve seat 1330 may, alternatively, be separately manufactured, positioned inwardly of the stop member 1338 and, if desired, secured to the housing 1302 with adhesive.

There are a variety of advantages associated with the main check valve 1300. For example, compression of the valve seat 1330 in the exemplary main check valve 1300 is a function of only two dimensions, i.e. the thickness of the valve seat and the height H of the stop member 1338, as compared to the three dimensions associated with the main check valve 107 illustrated in FIGS. 1-5. It is, therefore, substantially less likely that a tolerance stack-up will result in over-compression of the valve seat 1330, as compared to the main check valve 107. Additionally, even if there is some over-compression of the valve seat 1330, the valve seat will not impinge the valve element shaft 1316 because the diameter of the opening 1333 is relatively large, while the gap 1340 will allow the valve seat to deform outwardly, thereby preventing the formation of an irregular sealing surface.

Turning to the curved seal 1366, the curved seal reduces the contact area between the valve seat 1330 and the valve element head 1318, which in turn increases the sealing pressure, as compared to a sealing arrangement that has two flat surfaces. The curved seal 1366 also eliminates the adhesion force associated with flat on flat contact surfaces. The scuffing issue is also obviated because the valve element head 1318 does not have sharp lateral edges that come into contact with the valve seat 1330, which reduces stress risers.

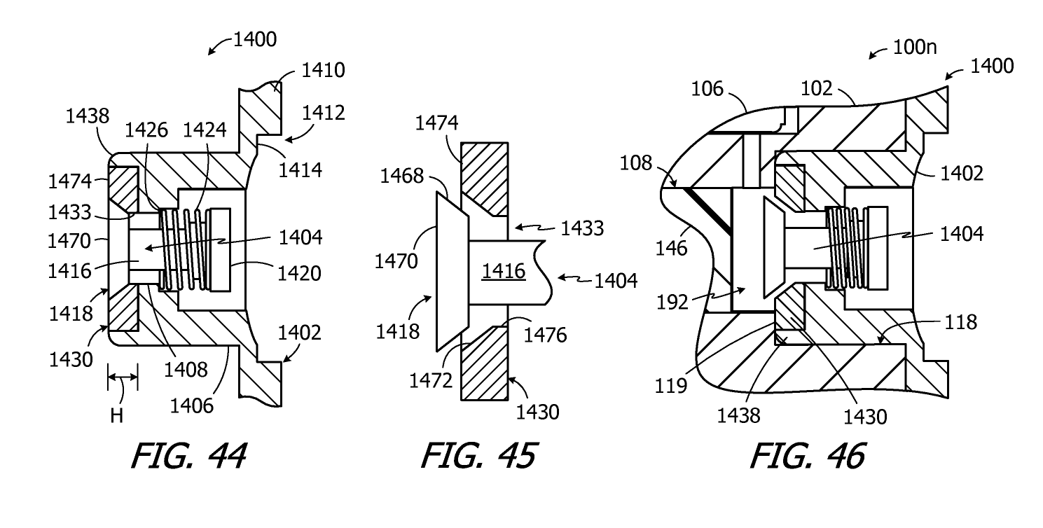

A main check valve in accordance with one embodiment of a present invention is generally represented by reference numeral 1400 in FIG. 44. The exemplary main check valve 1400 may be incorporated into a variety of fluid transfer devices. One such device is the fluid transfer device 100n illustrated in FIG. 46. The fluid transfer device 100n is essentially identical to the fluid transfer device 100, but for the substitution of main check valve 1400 for main check valve 107, and similar elements are represented by similar reference numerals.

As illustrated for example in FIG. 44, the main check valve 1400 includes a housing 1402 and a valve element 1404 that is movable relative to the housing 1402. The exemplary housing 1402 has a generally cylindrical fluid flow portion 1406, with a fluid lumen 1408 that is opened and closed by the valve element 1404, and a mounting portion 1410 that is used to secure the main check valve 1400 to a fluid transfer device or other structure. In the illustrated embodiment, which is configured for use with a cylindrical fluid transfer device, the mounting portion 1410 is disk-shaped. In other embodiments, the mounting portion 1410 may be resized, reshaped or omitted altogether. The mounting portion 1410 is also configured to receive an inlet tube (note tube 184 in FIGS. 1-5) and, accordingly, has a recess 1412, with a shoulder 1414 that is configured to receive the inlet tube. A filter (not shown) may be positioned within the recess 1412 between the inlet tube and the shoulder 1414. The recess 1412 and shoulder 1414 may, alternatively, be associated with the fluid flow portion 1406, or both the fluid flow portion and the mounting portion 1410, in other implementations of the main check valve 1400.

The exemplary valve element 1404 (or “plunger”) includes a shaft 1416 and a head 1418. A spring retainer 1420 is secured to the valve element 1404. More specifically, the spring retainer 1420 is secured to the end of the shaft 1416 opposite the head 1418 and may, for example, be press fit onto the shaft. The valve element 1404 is biased to the closed position illustrated in FIG. 44 by a spring 1424 (e.g. a coil spring) or other suitable biasing device. One end of the spring 1424 abuts a shoulder 1426 on the housing fluid flow portion 1406, and the other end of the spring abuts the spring retainer 1420. An elastomeric valve seat 1430, which has a central opening 1433 through which fluid passes, is positioned between the valve element head 1418 and the end of the housing fluid flow portion 1406.

The fluid lumen 1408 may be sized based on the desired fluid flow characteristics of the valve 1400 and, in the illustrated embodiment is about 0.019 inch to about 0.035 inch in diameter. The valve seat opening 1433 is the same size as fluid lumen 1408 in the illustrated embodiment, although it may be larger, in order to prevent the valve seat 1430 from interfering with fluid flow. Another factor associated with the size of the valve seat opening 1433 is the size of the valve element shaft 1416. More specifically, the opening 1433 should be sized such that, in the event that the valve seat 1430 is overcompressed and/or is off-center, the valve seat will be not impinge the valve element shaft 1416. To that end, the exemplary valve element shaft 1416 is about 0.014 inch to about 0.026 inch in diameter, and the exemplary valve seat opening 1433 will be about 0.024 inch to about 0.045 inch in diameter.

The exemplary main check valve 1400 is configured such that ullage (i.e. unfilled space) within the pump chamber of the associated fluid transfer device is eliminated or at least substantially eliminated. To that end, the valve element 1404 and the valve seat 1430 are configured such that the valve element head 1418 will not be located within the pump chamber of the associated fluid transfer device when the valve 1400 is closed. This may be accomplished in a variety of ways. In the exemplary embodiment, and as illustrated in FIG. 45, the valve element head 1418 is frusto-conical in shape and has a side surface 1468 that tapers inwardly from the forward surface 1470. The valve seat opening 1433 includes a frusto-conical portion 1472, which is essentially the same size and shape as the valve element head 1418 and extends downwardly from the forward surface 1474, and a small cylindrical portion 1476. So configured, the valve element head 1418 will nest within the valve seat 1430, with their forward surfaces 1470 and 1474 flush with one another, when the valve is in the closed state illustrated in FIG. 44.

The exemplary main check valve 1400 may, in some implementations, also be provided with apparatus that prevents over-compression of the elastomeric valve seat 1430. More specifically, the housing 1402 includes a stop member 1438 that extends from the end of flow portion 1406. The exemplary stop member 1438 is a rigid, substantially annular structure that is integral with the flow portion 1406 and has a height H that is equal to, or is slightly less than (e.g. about 0.003 inch less than), the thickness of the elastomeric valve seat 1430. The stop member 1438, which is located outwardly of the valve seat 1430, limits movement of the main check valve 1400 relative to the structure into which the main check valve is inserted. More specifically, in the exemplary fluid transfer device 100n illustrated in FIG. 46, the stop member 1438 engages the end wall 119 of the valve recess 118. It should also be noted that the stop member 1438 may define a continuous perimeter (as shown) or, alternatively, be composed of a plurality of spaced elements that together define a discontinuous perimeter. The stop member 1438, whether continuous or discontinuous, may also have an overall shape other than the illustrated substantially annular shape.

With respect to manufacturing and materials, the exemplary housing 1402 is a machined part and suitable materials for the housing include, but are not limited to, titanium, titanium alloys, stainless steel (e.g. 316L stainless steel), cobalt-nickel alloys, and refractory metals such as tantalum. The valve element 1404 may also be machined and suitable materials for the machined valve element include, but are not limited to, those described above in the context of the housing 1402. Alternatively, as discussed in greater detail below, the valve element 1404 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1430 include, but are not limited to, elastomers such as silicone rubber, latex rubber, urethane, butyl rubber, and isoprene. The valve seat 1430 may be molded directly onto the housing 1402 by way of a co-molding (or “insert molding”) process. Here, the housing 1402 is clamped into a mold that includes a cavity in the shape of the valve seat 1430, and the silicone rubber or other material is then injected into the cavity. A primer may be used to insure that the injected material sticks to the housing 1402. Such co-molding is advantageous because it eliminates the tolerance stack-up that is associated with the assembly of separately manufactured parts. Nevertheless, the valve seat 1430 may, alternatively, be separately manufactured, positioned inwardly of the stop member 1438 and, if desired, secured to the housing 1402 with adhesive.

There are a variety of advantages associated with the main check valve 1400. For example, compression of the valve seat 1430 in the exemplary main check valve 1400 is a function of only two dimensions, i.e. the thickness of the valve seat and the height H of the stop member 1438, as compared to the three dimensions associated with the main check valve 107 illustrated in FIGS. 1-5. It is, therefore, substantially less likely that a tolerance stack-up will result in over-compression of the valve seat 1430, as compared to the main check valve 107.

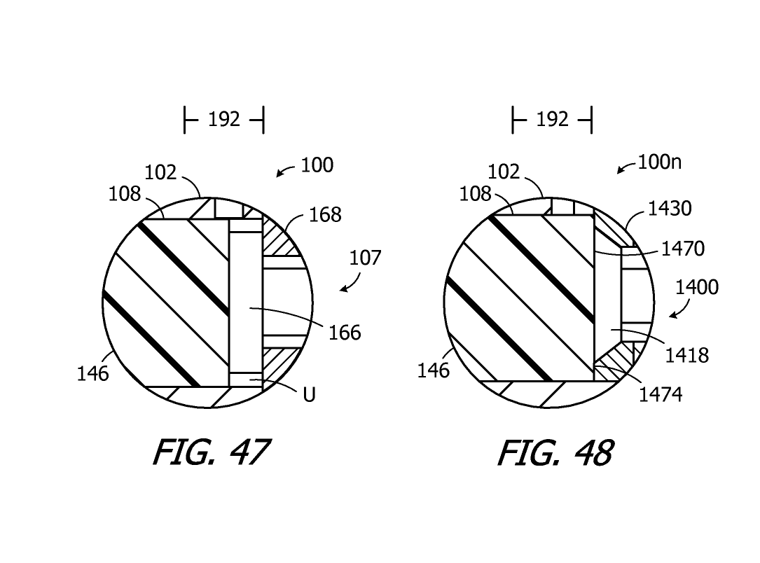

There are also a number of advantages related to the elimination of ullage. Referring first to FIG. 47, which illustrates a portion of the fluid transfer device 100 illustrated in FIGS. 1-5, the valve element head 166 is located within the pump chamber 192 at the end of the piston bore 108. This results in ullage U that extends around the perimeter of the valve element head 166 when the main check valve 107 is closed and the armature piston 146 abuts the valve element head. Should air enter the ullage U, movement of the armature piston 146 to the position illustrated in FIG. 3 may not result in a sufficient decrease in pressure within the pump chamber 192 to open the main check valve 107. Additionally, movement of the armature piston 146 from the position illustrated in FIG. 3 to the position illustrated in FIG. 5 may not result in a sufficient increase in pressure within the pump chamber 192 to open the bypass valve 106. Turning to FIG. 48, when the exemplary main check valve 1400 is closed, the valve element head 1418 is not within the pump chamber 192. The valve element head 1418 is, instead, nested within the valve seat 1430. The end portion of the armature piston 146 abuts the valve seat 1430 and occupies the entire pump chamber 192. As a result, there is no ullage associated with the valve element head 1418 and valve seat 1430.

A main check valve in accordance with one embodiment of a present invention is generally represented by reference numeral 1500 in FIG. 49. The exemplary main check valve 1500 may be incorporated into a variety of fluid transfer devices. One such device is the fluid transfer device 100o illustrated in FIG. 51. The fluid transfer device 100o is essentially identical to the fluid transfer device 100, but for the substitution of main check valve 1500 for main check valve 107, and similar elements are represented by similar reference numerals.

As illustrated for example in FIG. 49, the main check valve 1500 includes a housing 1502 and a valve element 1504 that is movable relative to the housing 1502. The exemplary housing 1502 has a generally cylindrical fluid flow portion 1506, with a fluid lumen 1508 that is opened and closed by the valve element 1504, and a mounting portion 1510 that is used to secure the main check valve 1500 to a fluid transfer device or other structure. In the illustrated embodiment, which is configured for use with a cylindrical fluid transfer device, the mounting portion 1510 is disk-shaped. In other embodiments, the mounting portion 1510 may be resized, reshaped or omitted altogether. The mounting portion 1510 is also configured to receive an inlet tube (note tube 184 in FIGS. 1-5) and, accordingly, has a recess 1512, with a shoulder 1514 that is configured to receive the inlet tube. A filter (not shown) may be positioned within the recess 1512 between the inlet tube and the shoulder 1514. The recess 1512 and shoulder 1514 may, alternatively, be associated with the fluid flow portion 1506, or both the fluid flow portion and the mounting portion 1510, in other implementations of the main check valve 1500.

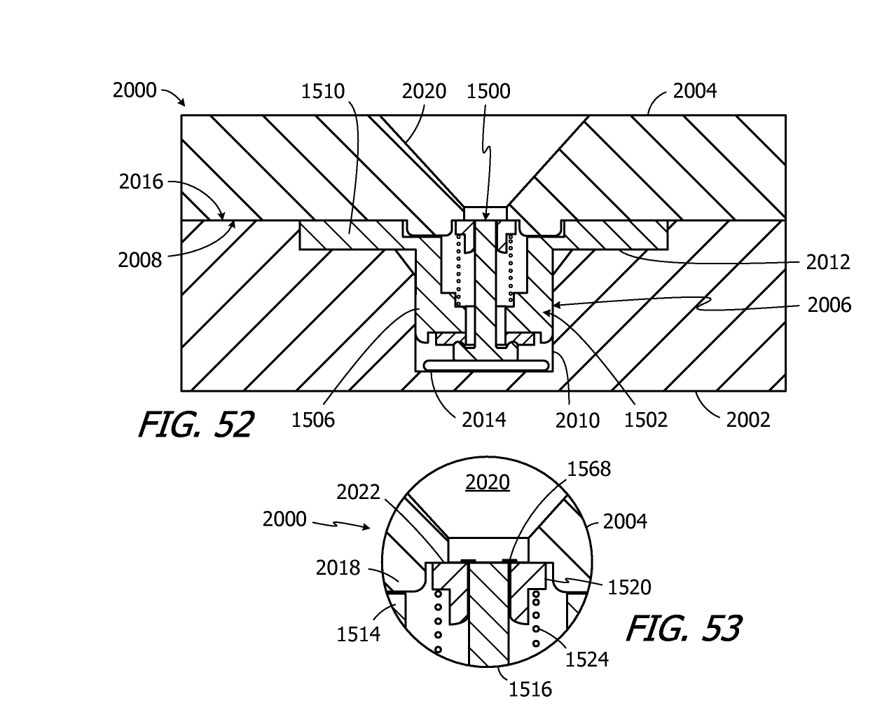

The exemplary valve element 1504 (or “plunger”) includes a shaft 1516 and a head 1518. A spring retainer 1520 is secured to the valve element 1504. More specifically, the spring retainer 1520 is secured to the end of the shaft 1516 opposite the head 1518. The shaft 1516 is relatively long and extends at least past the counter bore 1517 in the housing fluid flow portion 1506. The length of the shaft 1516 is essentially a function of the working length of the spring 1524 (discussed below). In the illustrated embodiment, the shaft 1516 also extends through inlet tube recess 1512 such that the shaft end 1516a is aligned the rearward surface of the housing 102 when the valve is in the closed state illustrated in FIG. 49. The exemplary shaft 1516 is also relatively thin, e.g. has a relatively small diameter. By way of example, the effective slenderness ratio (“ESR”) of the shaft 1516 is about 46.8, as compared to the 23.2 ESR of the valve element shaft 162 (FIGS. 1-5). As used herein, a relatively long, thin shaft is a shaft with an ESR that is at least 30.0. The spring retainer 1520 may, for example, be slip fit over and welded to the shaft 1516 in the manner described below in the context of FIGS. 52 and 53.

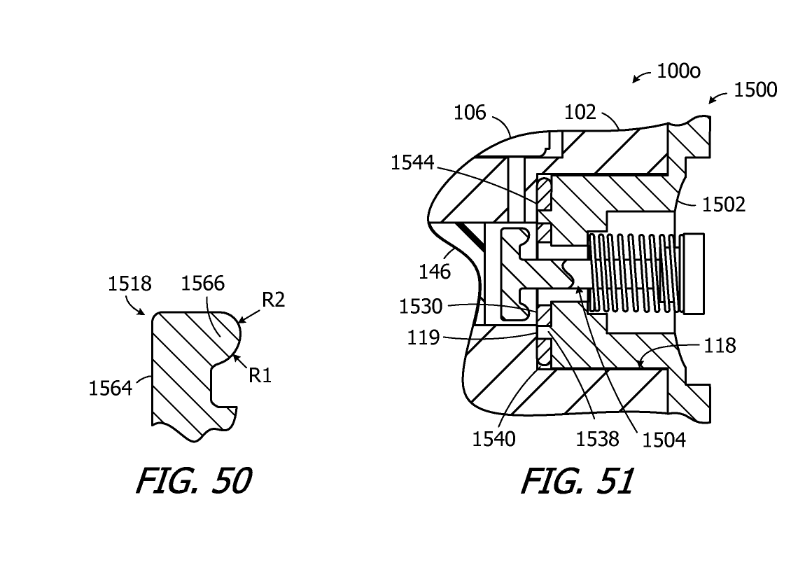

The head 1518 has a main portion 1564 and a curved seal 1566 that is semi-circular or otherwise curved in cross-section and protrudes from the main portion. The seal 1566 is rigid and engages the elastomeric valve seat 1530 (discussed below) when the valve 1500 is closed. Additionally, in the illustrated embodiment, the curved seal 1566 has an overall circular shape that engages a circular portion of the valve seat 1530 as well as the complex radius cross-sectional shape illustrated in FIG. 50, where R1 is about 40% greater than R2.

The valve element 1504 is biased to the closed position illustrated in FIG. 49 by a spring 1524 (e.g. a coil spring) or other suitable biasing device. One end of the spring 1524 abuts a shoulder 1526 on the housing fluid flow portion 1506, and the other end of the spring abuts the spring retainer 1520. The spring 1524 in the exemplary embodiment is also relatively soft, i.e. has a relatively low spring constant. Because the force (F) generated by a spring is equal to the spring constant (k) multiplied by displacement (x), or F=kx, a relatively soft spring must also be relatively long in order to generate the same spring force as a relatively stiff spring. For example, a relatively soft spring with a spring constant that is ½ of that of a relatively stiff spring will have to be compressed twice as much as the relatively stiff spring to generate the same spring force. The relevant compression in the context of the present valves is the compression from the uncompressed length (or “free length”) to the length after the valve has been assembled and is in the closed state (or “working length”) because the force associated with the free length to working length compression dictates the amount of force that is required to open the valve. Thus, in the example above, the relatively soft spring would have to be compressed twice as much as the relatively stiff spring to generate the same spring force at its working length. The benefits of the relatively long, soft spring are discussed below.

In the exemplary valve 1500, the relatively long, soft spring 1524 has a nominal spring constant of about 0.025 lbf/inch, a free length of about 0.108 inch, and a working length of about 0.050 inch. As such, the free length to working length ratio is about 2.2. By way of comparison, the spring in the main check valve 107 (FIGS. 1-5) has a nominal spring constant of about 0.17 lbf/inch and a free length to working length ratio is about 1.5.

An elastomeric valve seat 1530 is positioned between the valve element head 1518 and the end of the housing fluid flow portion 1506. The exemplary valve seat 1530 is sized and shaped such that it will be engaged by the valve element head 1518, but not by the housing of the associated fluid transfer device, e.g. the fluid transfer device 100o. The valve seat 1530 also has a generally annular shape, a central opening 1533 through which fluid passes, and a sealing surface (i.e. the surface engaged by the valve element head 1518) that is flat.

The fluid lumen 1508 may be sized based on the desired fluid flow characteristics of the valve 1500 and, in the illustrated embodiment is about 0.010 inch to about 0.019 inch in diameter. The valve seat opening 1533 will typically be at least the same size as fluid lumen 1508, and is larger in the illustrated embodiment, in order to prevent the valve seat 1530 from interfering with fluid flow. Another factor associated with the size of the valve seat opening 1533 is the size of the valve element shaft 1516. More specifically, the opening 1533 should be sized such that, in the event that the valve seat 1530 is overcompressed and/or is off-center, the valve seat will be not impinge the valve element shaft 1516. To that end, the exemplary valve element shaft 1516 is about 0.014 inch to about 0.026 inch in diameter, and the exemplary valve seat opening 1533 will be about 0.024 inch to about 0.045 inch in diameter.

As illustrated for example in FIGS. 49B and 49C, the exemplary valve seat 1530 includes a non-stick surface 1531 (or “release layer”) on the side of the valve seat that abuts the valve element head 1518. The non-stick surface 1531, which reduces the likelihood that the valve element head 1518 will stick to the valve seat 1530, may be in the form of a layer of silicon suboxide (SiOₓC_{y}, where x<2 and y<1) such as, for example, SiO_{1.7}C_{0.4}. The non-stick surface 1531 is relatively thin and plasma deposition (or other suitable techniques) may be used to deposit the silicon suboxide onto the valve seat 1530. The thickness of a relatively thin layer of silicon suboxide is about 0.1 μm to about 10 μm, and the non-stick surface 1531 in the illustrated embodiment is about 0.3 μm to about 0.8 μm thick. The non-stick surface 1531 may also cover the entire top surface of the valve seat 1530, only that part of the main portion top surface that would otherwise be engaged by the valve element head 15818, or something in between. The non-stick surface 1531 may, in other implementations, be a layer of SiO₂ that is about 0.1 μm to about 10 μm thick and formed by an oxygenated plasma treatment of the valve seat 1530. The pull-off adhesion of the non-stick material should be less than about 0.5 psi in those instances where the valve seat opening is about 0.040 inch. It should also be noted here that any of the valve seats and seals described above or below, or at least the portions thereof that come into contact with a valve element head or a rigid valve seat, may include the non-stick surface, and that the valve 1500 may be provided with an otherwise identical valve seat without the non-stick surface.

An elastomeric gasket 1544, which is a separate structural element from the valve seat 1530, may be used to form a seal between the housing fluid flow portion 1506 and the housing 102 or other structure into which the main check valve 1500 is inserted. The exemplary gasket 1544 has a base 1532 and raised seal 1535 that protrudes from the base, as is shown in FIG. 49A. The exemplary base 1532 is annularly shaped and is rectangular in cross-section, while exemplary raised seal 1535 has a circular shape and is semi-circular or otherwise curved in cross-section. The outer diameter of the exemplary gasket 1544 is slightly less (e.g. about 0.005 inch to about 0.020 inch in the exemplary embodiment) than the outer diameter of the housing fluid flow portion 1506. This difference produces a gap 1540 which allows the gasket 1544 to deform (or “extrude”) outwardly when compressed in the manner illustrated in FIG. 51.

The exemplary main check valve 1500 illustrated in FIG. 49 is also provided with apparatus that controls the compression of the gasket 1544. More specifically, the housing 1502 includes a stop member 1538 that extends from the end of the housing flow portion 1506. The exemplary stop member 1538 is a rigid, substantially annular structure that is integral with the housing flow portion 1506 and has a height H that is equal to, or is slightly less than (e.g. about 0.003 inch less than), the thickness of the gasket 1544. The stop member 1538, which is located between the valve seat 1530 and the gasket 1544, limits movement of the main check valve 1500 relative to the structure into which the main check valve is inserted. More specifically, in the exemplary fluid transfer device 100o illustrated in FIG. 51, the stop member 1538 engages the end wall 119 of the valve recess 118. It should also be noted that the stop member 1538 may define a continuous perimeter (as shown) or, alternatively, be composed of a plurality of spaced elements that together define a discontinuous perimeter. The stop member 1538, whether continuous or discontinuous, may also have an overall shape other than the illustrated substantially annular shape.

Additionally, in some embodiments, the stop member 1538 may include a plurality of small holes (not shown) that extend through the stop member in the radial direction (e.g. 4 holes with 90 degree spacing) that are used when the valve seat 1530 and gasket 1544 are simultaneously co-molded onto the housing 1502. The co-molding holes are discussed below in the context of the co-molding process.

The outer diameter of the exemplary gasket 1544 is slightly less (e.g. about 0.005 inch to about 0.020 inch in the exemplary embodiment) than the outer diameter of the housing fluid flow portion 1506. This difference produces a gap 1540 which allows the gasket 1544 to deform (or “extrude”) outwardly when compressed in the manner illustrated in FIG. 51.

With respect to manufacturing and materials, the exemplary housing 1502 is a machined part and suitable materials for the housing include, but are not limited to, titanium, titanium alloys, stainless steel (e.g. 316L stainless steel), cobalt-nickel alloys, and refractory metals such as tantalum. The valve element 1504 may also be machined and suitable materials for the machined valve element include, but are not limited to, those described above in the context of the housing 1502. Alternatively, as discussed in greater detail below, the valve element 1504 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1530 and gasket 1544 include, but are not limited to, elastomers such as silicone rubber, latex rubber, urethane, butyl rubber, and isoprene.

The valve seat 1530 and gasket 1544 may be molded directly onto the housing 1502 by way of a co-molding (or “insert molding”) process. Here, the housing 1502 is clamped into a mold that includes cavities in the shape of the valve seat 1530 and gasket 1544, and the silicone rubber or other material is then injected into the cavities. A primer may be used to insure that the injected material sticks to the housing 1502. In those instances where the co-molding holes in the stop member 1538 are present, the material need only be injected into one of the cavities because it will flow through the co-molding holes to the other. Such co-molding is advantageous because it eliminates the tolerance stack-up that is associated with the assembly of separately manufactured parts. Nevertheless, the valve seat 1530 and gasket 1544 may, alternatively, be separately manufactured, positioned inwardly and outwardly of the stop member 1538 and, if desired, secured to the housing 1502 with adhesive. Other aspects of the assembly process are discussed below in the context of FIGS. 52 and 53.

There are a variety of advantages associated with the main check valve 1500. For example, the main check valve 1500 avoids the above-described valve seat over-compression problems by configuring the valve seat 1530 such that it does not engage the housing of the fluid transfer device into which the valve is inserted. More specifically, as illustrated in FIG. 51, there is no assembly-related compression of the valve seat 1530 when the exemplary valve 1500 is inserted into the fluid transfer device housing 102. The only force applied to the valve seat 1530 is that associated with the spring 1524. Additionally, even if there is some over-compression of the valve seat 1530, the valve seat will not impinge the valve element shaft 1516 because the diameter of the opening 1533 is relatively large. The small diameter of the valve element shaft 1516 further reduces the likelihood that the shaft will be impinged by the valve seat 1530.

Turning to the gasket, compression of the gasket 1544 in the exemplary main check valve 1500 is a function of only two dimensions, i.e. the thickness of the valve seat and the height H of the stop member 1538, as compared to the three dimensions associated with the main check valve 107 illustrated in FIGS. 1-5. It is, therefore, substantially less likely that a tolerance stack-up will result in over-compression of the gasket 1544, as compared to the main check valve 107. Additionally, even if there is some over-compression of the gasket 1544, the gap 1540 will allow the gasket to deform outwardly.

With respect to the relationship between the valve seat 1530 and the gasket 1544, the valve seat and gasket are separate structural elements and are spaced from one another. Compression (and over-compression) of the gasket 1544 will not, therefore, substantially effect the elastomeric valve seat 1530. Even in those instances where the valve seat 1530 and gasket 1544 are connected to one another by a small amount of elastomeric material that remains in the aforementioned co-molding holes in the stop member 1538, this connection will not result in compression (and over-compression) of the gasket substantially effecting the elastomeric valve seat.

The raised seal 1535 will be under compression when the main check valve 1500 is inserted into the valve recess 118 of the housing 102. Compressing the raised seal 1535 is advantageous because silicone and other elastomeric materials tend to tear when in tension. The raised seal 1535 also increases sealing pressure in a manner that is relatively easy to manufacture.

Turning to the curved seal 1566, the curved seal reduces the contact area between valve seat 1530 and the valve element head 1518, which in turn increases the sealing pressure, as compared to a sealing arrangement that has two flat surfaces. The curved seal 1566 also eliminates the adhesion force associated with flat on flat contact surfaces. The scuffing issue is also obviated because the valve element head 1518 does not have sharp lateral edges that come into contact with the valve seat 1530, which reduces stress risers.

There are also a number of advantages associated with the use of the relatively soft and long spring 1524. For example, a relatively soft spring is less sensitive to variations in the working length that can be caused by tolerance stack-up. As such, it is less likely that the working length of the spring 1524 will have to be adjusted, in response to tolerance stack-up, during assembly of the valve 1500. Additionally, when the spring 1524 is compressed as the valve 1500 opens, the coils in a relatively long spring are less likely to compress to the point at which the spring blocks fluid flow.

As alluded to above, one method of assembling the valve 1500 includes slip fitting the spring retainer 1520 over the valve element shaft 1516 and then welding the spring retainer to the shaft. One example of an assembly fixture that may be used in conjunction with such a method is generally represented by reference numeral 2000 in FIGS. 52 and 53. The assembly fixture 2000 includes a base 2002 and a cover 2004. The base 2002 is configured to receive, for example, the valve housing 1502 and, to that end, has a bore 2006 that extends (in the illustrated orientation) downwardly from the top surface 2008. The bore 206 includes a relatively long, narrow portion 2010 for the housing fluid flow portion 1506 as well as a relatively short, wide portion 2012 for the housing mounting portion 1510. The depth of the bore 2006 may be adjusted, as necessary, through the use of one or more shims 2014. The cover 2004 includes a bottom surface 2016 that rests on the base top surface 2008, an annular abutment 2018 that rests on the recess shoulder 1514, and an aperture 2020, with a frusto-conical portion and a cylindrical portion, that provides access to the shaft 1516 and spring retainer 1520. There is also a shoulder 2022, which rests on the spring retainer 1520, between the annular abutment 2018 and the aperture 2020. In the context of the exemplary valve 1500, where the shaft 1516 extends to the end to the housing 1502, the cover bottom surface 2008 and shoulder 2022 are coplanar.

At the beginning of the assembly process, the fixture cover 2004 is removed from the base 2002 and the valve 1500, less the spring retainer 1520, is positioned within the fixture base in the manner illustrated in FIG. 52. At this point, the spring 1524 will be uncompressed and substantially longer than (e.g. twice as long as) the shaft 1516. The spring retainer 1520 will then be placed on the free end of the spring 1524. The fixture cover 2004 may be positioned over the base 2002, with the shoulder 2022 aligned with the spring retainer 1520, and then lowered to the position illustrated in FIG. 52. The spring 1524 will be compressed until the spring retainer is slip fit (as opposed to press fit) over the valve element shaft 1516 and the rearward surfaces of the valve element shaft and spring retainer are aligned with one another, as best seen in FIG. 53.

Next, the spring retainer 1520 will be secured to the shaft 1516, in a manner other than press fitting, by way of the fixture aperture 2020. For example, a spot welding tool may be inserted through the aperture 2020 and used to form spot welds 1568 around the perimeter of shaft 1516 at the shaft/spring retainer intersection. Other types of welding may also employed. Alternatively, adhesive or crimping may be used to secure the spring retainer 1520 to the shaft 1516.

There are a number of advantages associated with the above-described assembly fixture 2000 and assembly process. For example, as compared to assembly processes that employ a press fit, the present slip fit and secure (e.g. by welding) process facilitates the use of a valve element shaft that is longer and of smaller diameter because the amount of force required to achieve a press fit will bend such a shaft. The use of a longer and thinner shaft will, as described above, facilitate the use of a longer spring and decrease the likelihood that the valve seat will impinge the valve shaft.

It should also be noted here that any of the main check valves described above in the context of FIGS. 1-48 may be modified that the so as to employ a relatively long, thin valve element shaft and the relatively long, soft spring, and be assembled in the manner described in the manner described in the context of FIGS. 52 and 53.

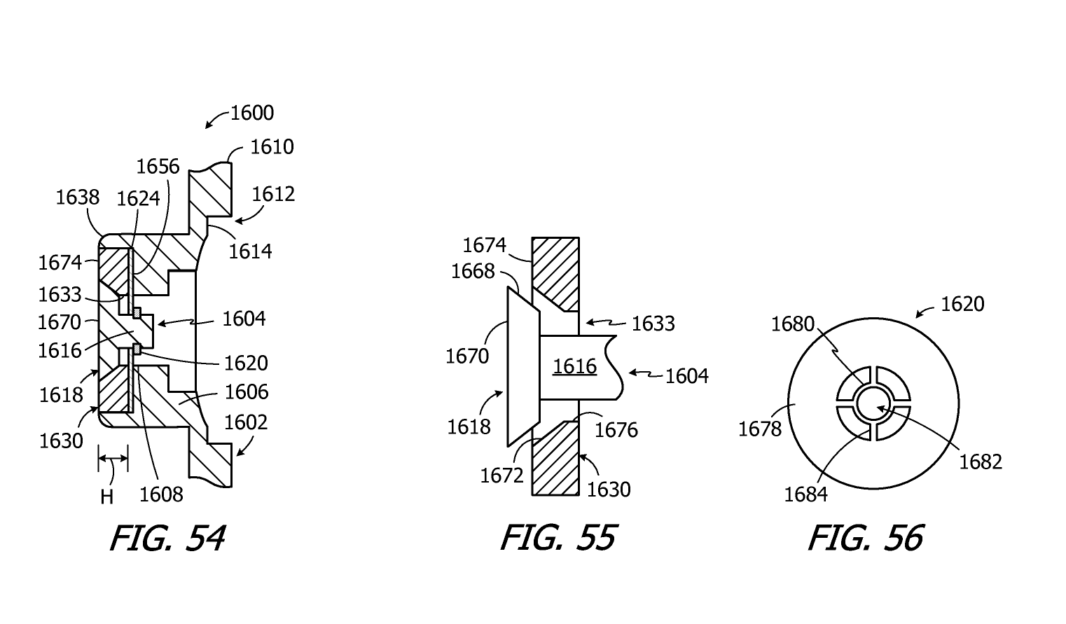

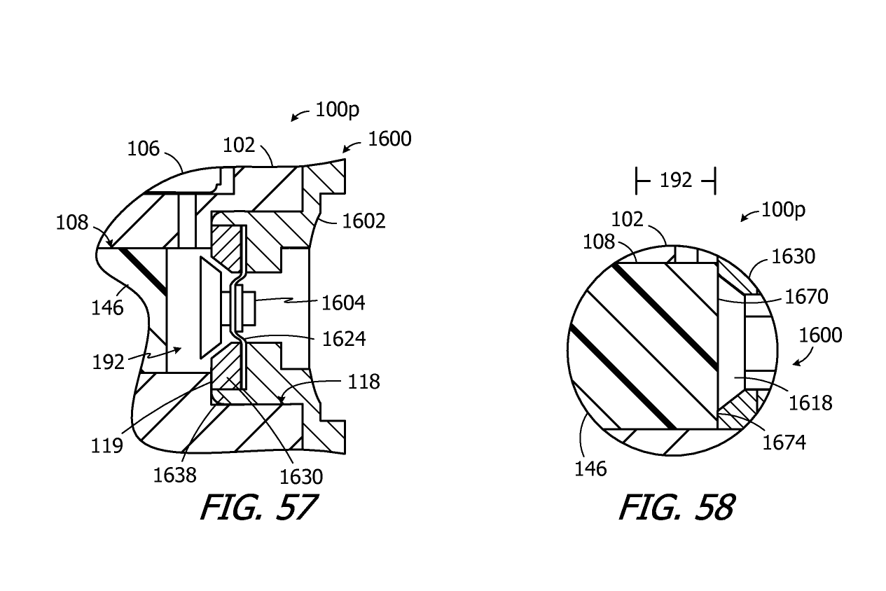

A main check valve in accordance with one embodiment of a present invention is generally represented by reference numeral 1600 in FIG. 54. The exemplary main check valve 1600 may be incorporated into a variety of fluid transfer devices. One such device is the fluid transfer device 100p illustrated in FIG. 57. The fluid transfer device 100p is essentially identical to the fluid transfer device 100, but for the substitution of main check valve 1600 for main check valve 107, and similar reference numerals are represent similar elements.

As illustrated for example in FIG. 54, the main check valve 1600 includes a housing 1602 and a valve element 1604 that is movable relative to the housing 1602. The exemplary housing 1602 has a generally cylindrical fluid flow portion 1606, with a fluid lumen 1608 that is opened and closed by the valve element 1604, and a mounting portion 1610 that is used to secure the main check valve 1600 to a fluid transfer device or other structure. In the illustrated embodiment, which is configured for use with a cylindrical fluid transfer device, the mounting portion 1610 is disk-shaped. In other embodiments, the mounting portion 1610 may be resized, reshaped or omitted altogether. The mounting portion 1610 is also configured to receive an inlet tube (note tube 184 in FIGS. 1-5) and, accordingly, has a recess 1612, with a shoulder 1614 that is configured to receive the inlet tube. A filter (not shown) may be positioned within the recess 1612 between the inlet tube and the shoulder 1614. The recess 1612 and shoulder 1614 may, alternatively, be associated with the fluid flow portion 1606, or both the fluid flow portion and the mounting portion 1610, in other implementations of the main check valve 1600.

The exemplary valve element 1604 (or “plunger”) includes a shaft 1616 and a head 1618 and a spring retainer 1620 (e.g. a snap ring) is secured to the shaft. The valve element 1604 is biased to the closed position illustrated in FIG. 54 by a flat (or “planar”) spring 1624 or other suitable biasing device. The planar spring 1624 is positioned between housing surface 1656 and an elastomeric valve seat 1630 (discussed in more detail below). The exemplary valve seat 1630, which has a central opening 1633 through which fluid passes, is itself positioned between the valve element head 1618 and the planar spring 1624.

The spring retainer 1620 may be used to impart and adjust the preload on the planar spring 1624. For example, the differently sized spring retainers may be employed, or shims may be placed between the spring retainer 1620 and the planar spring 1624.

The planar spring is not limited to any particular configuration. For example, and referring to FIG. 56, the illustrated planar spring 1624 includes an outer ring 1678 that is coextensive with the valve seat 1630, an inner ring 1680 with an opening 1682 for the valve element shaft 1616, and a plurality of (e.g. four) deflectable members 1684 that connect the inner ring to the outer ring. Another example of a suitable planar spring is a spiral spring, which includes a spiral-shaped deflectable member that extends around the inner ring and connects inner ring to the outer ring.

The fluid lumen 1608 may be sized based on the desired fluid flow characteristics of the valve 1600 and, in the illustrated embodiment is about 0.019 inch to about 0.035 inch in diameter. The valve seat opening 1633 is the same size as fluid lumen 1608 in the illustrated embodiment, although it may be larger, in order to prevent the valve seat 1630 from interfering with fluid flow. Another factor associated with the size of the valve seat opening 1633 is the size of the valve element shaft 1616. More specifically, the opening 1633 should be sized such that, in the event that the valve seat 1630 is overcompressed and/or is off-center, the valve seat will be not impinge the valve element shaft 1616. To that end, the exemplary valve element shaft 1616 is about 0.014 inch to about 0.026 inch in diameter, and the exemplary valve seat opening 1633 will be about 0.024 inch to about 0.045 inch in diameter.

The exemplary main check valve 1600 is configured such that ullage within the pump chamber of the associated fluid transfer device is eliminated or at least substantially eliminated. To that end, the valve element 1604 and the valve seat 1630 are configured such that the valve element head 1618 will not be located within the pump chamber of the associated fluid transfer device when the valve 1600 is closed. This may be accomplished in a variety of ways. In the exemplary embodiment, and as illustrated in FIG. 55, the valve element head 1618 is frusto-conical in shape and has a side surface 1668 that tapers inwardly from the forward surface 1670. The valve seat opening 1633 includes a frusto-conical portion 1672, which is essentially the same size and shape as the valve element head 1618 and extends downwardly from the forward surface 1674, and a small cylindrical portion 1676. So configured, the valve element head 1618 will nest within the valve seat 1630, with their forward surfaces 1670 and 1674 flush with one another, when the valve is in the closed state illustrated in FIG. 54.

The exemplary main check valve 1600 may, in some implementations, also be provided with apparatus that prevents over-compression of the elastomeric valve seat 1630. More specifically, the housing 1602 includes a stop member 1638 that extends from the end of flow portion 1606. The exemplary stop member 1638 is a rigid, substantially annular structure that is integral with the flow portion 1606 and has a height H that is equal to, or is slightly less than (e.g. about 0.003 inch less than), the thickness of the elastomeric valve seat 1630. The stop member 1638, which is located outwardly of the valve seat 1630, limits movement of the main check valve 1600 relative to the structure into which the main check valve is inserted. More specifically, in the exemplary fluid transfer device 100p illustrated in FIG. 57, the stop member 1638 engages the end wall 119 of the valve recess 118. It should also be noted that the stop member 1638 may define a continuous perimeter (as shown) or, alternatively, be composed of a plurality of spaced elements that together define a discontinuous perimeter. The stop member 1638, whether continuous or discontinuous, may also have an overall shape other than the illustrated substantially annular shape.

With respect to manufacturing and materials, the exemplary housing 1602 is a machined part and suitable materials for the housing include, but are not limited to, titanium, titanium alloys, stainless steel (e.g. 316L stainless steel), cobalt-nickel alloys, and refractory metals such as tantalum. The valve element 1604 may also be machined and suitable materials for the machined valve element include, but are not limited to, those described above in the context of the housing 1602. Alternatively, as discussed in greater detail below, the valve element 1604 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1630 include, but are not limited to, elastomers such as silicone rubber, latex rubber, urethane, butyl rubber, and isoprene.

During assembly, the planar spring 1624 is placed against the housing surface 1656. The valve seat 1630 may be molded directly onto the housing 1602 and planar spring 1624 by way of a co-molding (or “insert molding”) process. Here, the housing 1602 is clamped into a mold that includes a cavity in the shape of the valve seat 1630, and the silicone rubber or other material is then injected into the cavity. A primer may be used to insure that the injected material sticks to the housing 1602 and planar spring 1624. Such co-molding is advantageous because it eliminates the tolerance stack-up that is associated with the assembly of separately manufactured parts. Nevertheless, the valve seat 1630 may, if desired, be separately manufactured and secured to the housing 1602 and planar spring 1624 with adhesive. The valve element 1604 may then be secured in place by connecting the spring retainer 1620 to the shaft 1616.

There are a variety of advantages associated with the main check valve 1600. For example, compression of the valve seat 1630 in the exemplary main check valve 1600 is a function of only two dimensions, i.e. the thickness of the valve seat and the height H of the stop member 1638, as compared to the three dimensions associated with the main check valve 107 illustrated in FIGS. 1-5. It is, therefore, substantially less likely that a tolerance stack-up will result in over-compression of the valve seat 1630, as compared to the main check valve 107.

There are also a number of advantages related to the elimination of ullage. Referring first to FIG. 47, which illustrates a portion of the fluid transfer device 100 illustrated in FIGS. 1-5, the valve element head 166 is located within the pump chamber 192 at the end of the piston bore 108. This results in ullage U that extends around the perimeter of the valve element head 166 when the main check valve 107 is closed and the armature piston 146 abuts the valve element head. Should air enter the ullage U, movement of the armature piston 146 to the position illustrated in FIG. 3 may not result in a sufficient decrease in pressure within the pump chamber 192 to open the main check valve 107. Additionally, movement of the armature piston 146 from the position illustrated in FIG. 3 to the position illustrated in FIG. 5 may not result in a sufficient increase in pressure within the pump chamber 192 to open the bypass valve 106. Turning to FIG. 58, when the exemplary main check valve 1600 is closed, the valve element head 1618 is not within the pump chamber 192. The valve element head 1618 is, instead, nested within the valve seat 1630. The end portion of the armature piston 146 abuts the valve seat 1630 and occupies the entire pump chamber 192. As a result, there is no ullage.

Turning to the planar spring 1624, the planar spring reduces the overall length of the valve 1600 (as compared to an otherwise identical valve), thereby reducing the overall length of the associated fluid transfer device, which may result in a smaller implantable or otherwise ambulatory medical device. For example, the valve recess 118 in the fluid transfer device 100p (FIG. 57) is substantially shorter than the valve recess 118 in the fluid transfer device 100n (FIG. 46) because the fluid flow portion 1606 of the valve 1600 is substantially shorter than the fluid flow portion 1406 of the valve 1400. The shorter valve recess 118 results in a shorter fluid transfer device housing 102.

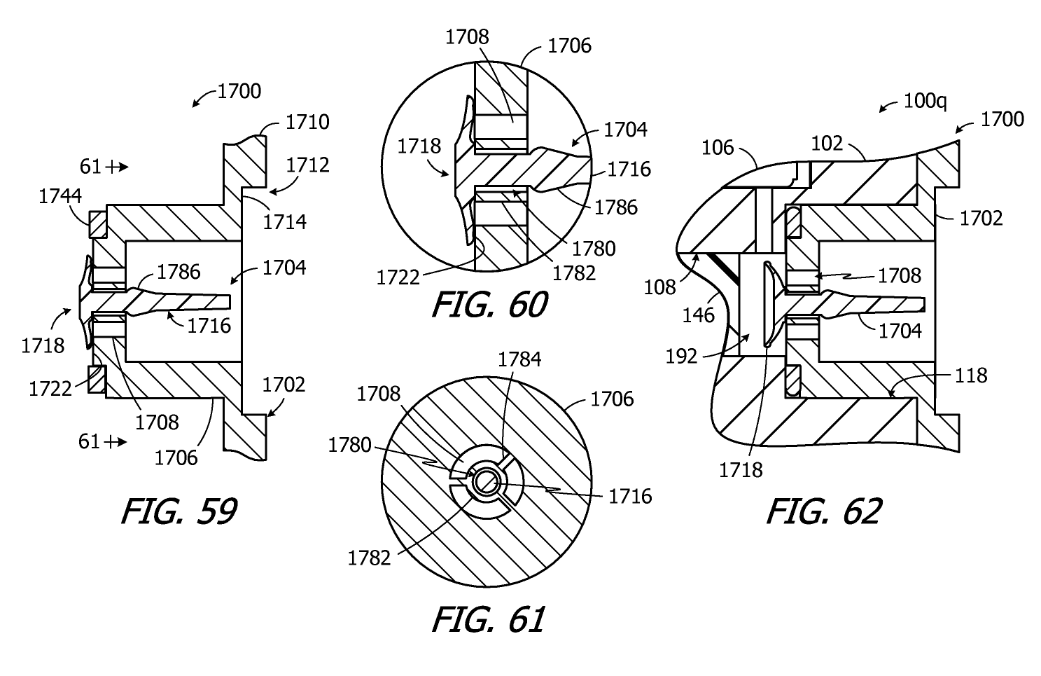

A main check valve in accordance with one embodiment of a present invention is generally represented by reference numeral 1700 in FIG. 59. The exemplary main check valve 1700 may be incorporated into a variety of fluid transfer devices. One such device is the fluid transfer device 100q illustrated in FIG. 62. The fluid transfer device 100q is essentially identical to the fluid transfer device 100, but for the substitution of main check valve 1700 for main check valve 107, and similar elements are represented by similar reference numerals.

As illustrated for example in FIGS. 59-61, the main check valve 1700 is an umbrella-type valve that includes a housing 1702 and a valve element 1704. The exemplary housing 1702 has a generally cylindrical fluid flow portion 1706, with a fluid lumen 1708 that is opened and closed by the valve element 1704 and valve element lumen 1780 that receives the valve element shaft 1716 (discussed below). The valve element lumen 1780 is defined by a tube 1782 that is held within the fluid lumen 1708 by supports 1784 (FIG. 61) that divide the fluid lumen into three portions. The exemplary housing also includes a mounting portion 1710 that is used to secure the main check valve 1700 to a fluid transfer device or other structure. In the illustrated embodiment, which is configured for use with a cylindrical fluid transfer device, the mounting portion 1710 is disk-shaped. In other embodiments, the mounting portion 1710 may be resized, reshaped or omitted altogether. The mounting portion 1710 is also configured to receive an inlet tube (note tube 184 in FIGS. 1-5) and, accordingly, has a recess 1712, with a shoulder 1714 that is configured to receive the inlet tube. A filter (not shown) may be positioned within the recess 1712 between the inlet tube and the shoulder 1714. The recess 1712 and shoulder 1714 may, alternatively, be associated with the fluid flow portion 1706, or both the fluid flow portion and the mounting portion 1710, in other implementations of the main check valve 1700.

The exemplary valve element 1704 includes a shaft 1716 that carries a head 1718, which is located at one end of the tube 1782, and an abutment 1786, which is located at the other end of the tube. As such, once the valve 1700 is assembled, the location of the shaft 1716 is essentially fixed and the orientation of the head determines whether the valve is open or closed. More specifically, the head 1718 is pivotable, due to the elastic properties of the material from which the valve element is formed, between the flat shape (or “closed orientation”) illustrated in FIGS. 59 and 60 and the cup shape (or “open orientation”) illustrated in FIG. 62. The head 1718 rests on the housing seal surface 1722 and covers the outlet of the fluid lumen 1708 when in the closed orientation. The head 1718 is also biased to the closed orientation shape is deflected to the open orientation when the pressure differential across the valve reaches the predetermined level (e.g. about 0.5 to 1.5 psid). More specifically, during operation of the fluid transfer device 100q, movement of the piston 146 from a position where it abuts the valve element head 1718 to the position illustrated in FIG. 62, where the piston is spaced from the valve element head, results in a decrease in pressure in the pump chamber 192. The reduction in pressure within the pump chamber 192 creates the pressure differential that opens the valve 1700. The head 1718 will snap back to the closed orientation, thereby closing the valve 1700, when the pressure differential drops below the predetermined level. This type of valve element may be obtained from, for example, Minivalve International, located in Oldenzaal, The Netherlands.

An elastomeric gasket 1744 may be used to form a seal between the housing fluid flow portion 706 and the housing 102 or other structure into which the main check valve 700 is inserted. The outer diameter of the exemplary gasket 1744 is slightly less (e.g. about 0.005 inch to about 0.020 inch in the exemplary embodiment) than the outer diameter of the fluid flow portion 1706. This difference produces a gap 1740 which allows the gasket 1744 to deform (or “extrude”) outwardly when compressed in the manner illustrated in FIG. 62.

With respect to manufacturing and materials, the exemplary housing 1702 is a machined part and suitable materials for the housing include, but are not limited to, titanium, titanium alloys, stainless steel (e.g. 316L stainless steel), cobalt-nickel alloys, and refractory metals such as tantalum. The valve element 1704 may be a molded, unitary structure, i.e. the shaft 1716 and head 1718 may be formed in a single mold from the same material. Suitable materials for the valve element 1704 and gasket 1744 include, but are not limited to, resilient materials such as elastomers such as silicone rubber, latex rubber, urethane, butyl rubber, and isoprene. During assembly, the valve element 1704 is simply pushed through the valve element tube 1782 until it reaches the position illustrated in FIG. 59.

There are a variety of advantages associated with the main check valve 1700. For example, as compared to the main check valve illustrated in FIGS. 1-5, the main check valve 1700 includes fewer parts, i.e. no valve seat, no spring and no spring retainer. This simplifies both manufacture and assembly, and also eliminates the many of the tolerance stack-up issues described above. Additionally, as the valve element 1704 may be formed from silicone rubber or other relative soft materials (as compared to, for example, titanium), the head 1718 will absorb any surface irregularities in the housing seal surface 1722 against which the head creates a seal. A molded valve element may also be easily reproduced from a mold, formed from materials with particular properties (e.g. hydrophobic properties), and manufactured less expensively, with fewer surface variations, than a machined valve element.

The valve element 1704 may also be reconfigured in such a manner that the overall length of the valve 1700 can be reduced (as compared to an otherwise identical valve), thereby reducing the overall length of the associated fluid transfer device. More specifically, the portion of the valve element shaft 1716 that carries is located upstream from the abutment 1786 may be removed which. The shortened valve element shaft 1716 facilitates the use of a shorter housing fluid flow portion 1706 which, in turn, facilitates the use of shorter valve recess 118 (and housing 102) in the fluid transfer device. A smaller fluid transfer device may result in a smaller implantable or otherwise ambulatory medical device.

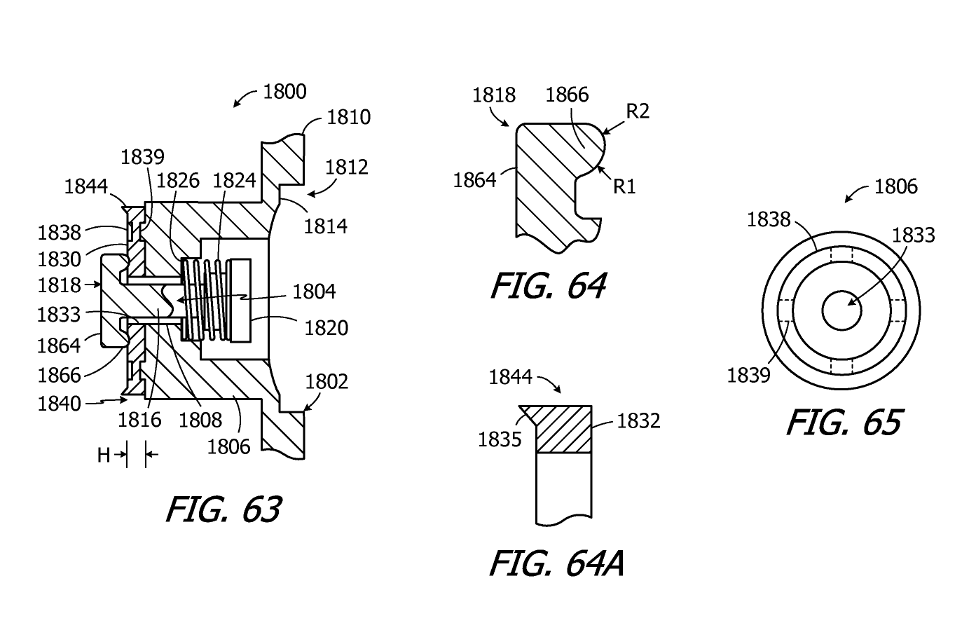

A main check valve in accordance with one embodiment of a present invention is generally represented by reference numeral 1800 in FIG. 63. The exemplary main check valve 1800 may be incorporated into a variety of fluid transfer devices. One such device is the fluid transfer device 100r illustrated in FIG. 66. The fluid transfer device 100r is essentially identical to the fluid transfer device 100, but for the substitution of main check valve 1800 for main check valve 107, and similar elements are represented by similar reference numerals.

As illustrated for example in FIG. 63, the main check valve 1800 includes a housing 1802 and a valve element 1804 that is movable relative to the housing 1802. The exemplary housing 1802 has a generally cylindrical fluid flow portion 1806, with a fluid lumen 1808 that is opened and closed by the valve element 1804, and a mounting portion 1810 that is used to secure the main check valve 1800 to a fluid transfer device or other structure. In the illustrated embodiment, which is configured for use with a cylindrical fluid transfer device, the mounting portion 1810 is disk-shaped. In other embodiments, the mounting portion 1810 may be resized, reshaped or omitted altogether. The mounting portion 1810 is also configured to receive an inlet tube (note tube 184 in FIGS. 1-5) and, accordingly, has a recess 1812, with a shoulder 1814 that is configured to receive the inlet tube. A filter (not shown) may be positioned within the recess 1812 between the inlet tube and the shoulder 1814. The recess 1812 and shoulder 1814 may, alternatively, be associated with the fluid flow portion 1806, or both the fluid flow portion and the mounting portion 1810, in other implementations of the main check valve 1800.

The exemplary valve element 1804 (or “plunger”) includes a shaft 1816 and a head 1818. A spring retainer 1820 is secured to the valve element 1804. More specifically, the spring retainer 1820 is secured to the end of the shaft 1816 opposite the head 1818 and may, for example, be press fit onto the shaft. The head 1818 has a main portion 1864 and a curved seal 1866 that is semi-circular or otherwise curved in cross-section and protrudes from the main portion. The seal 1866 may be rigid and engages the elastomeric valve seat 1830 (discussed below) when the valve 1800 is closed. Additionally, in the illustrated embodiment, the curved seal 1866 has an overall circular shape that engages a circular portion of the valve seat 1830 as well as the complex radius cross-sectional shape illustrated in FIG. 64, where R1 is about 40% greater than R2.

The valve element 1804 is biased to the closed position illustrated in FIG. 63 by a spring 1824 (e.g. a coil spring) or other suitable biasing device. One end of the spring 1824 abuts a shoulder 1826 on the housing fluid flow portion 1806, and the other end of the spring abuts the spring retainer 1820.

An exemplary elastomeric valve seat 1830 is positioned between the valve element head 1818 and the end of the housing fluid flow portion 1806. The valve seat 1830 is sized and shaped such that it will be engaged by the valve element head 1818, but will not be substantially compressed (or compressed at all) by the housing of the associated fluid transfer device, e.g. the fluid transfer device 100r. The valve seat 1830 also has a generally annular shape, a central opening 1833 through which fluid passes, and a sealing surface (i.e. the surface engaged by the valve element head 1818) that is flat.

The fluid lumen 1808 may be sized based on the desired fluid flow characteristics of the valve 1800 and, in the illustrated embodiment is about 0.019 inch to about 0.035 inch in diameter. The valve seat opening 1833 will typically be at least the same size as fluid lumen 1808 (as shown), or larger, in order to prevent the valve seat 1830 from interfering with fluid flow. Another factor associated with the size of the valve seat opening 1833 is the size of the valve element shaft 1816. More specifically, the opening 1833 should be sized such that, in the event that the valve seat 1830 is overcompressed and/or is off-center, the valve seat will be not impinge the valve element shaft 1816. To that end, the exemplary valve element shaft 1816 is about 0.014 inch to about 0.026 inch in diameter, and the exemplary valve seat opening 1833 will be about 0.024 inch to about 0.045 inch in diameter.

An elastomeric gasket 1844, which is a separate structural element from the valve seat 1830, may be used to form a seal between the housing fluid flow portion 1806 and the housing 102 or other structure into which the main check valve 1800 is inserted. The exemplary gasket 1844 has a base 1832 and raised seal 1835 that protrudes from the base, as is shown in FIG. 64A. The exemplary base 1832 is annularly shaped and is rectangular in cross-section, while exemplary raised seal 1835 has a circular shape and is triangular in cross-section. The outer diameter of the exemplary gasket 1844 is slightly less (e.g. about 0.005 inch to about 0.020 inch in the exemplary embodiment) than the outer diameter of the housing fluid flow portion 1806. This difference produces a gap 1840 which allows the gasket 1844 to deform (or “extrude”) outwardly when compressed in the manner illustrated in FIG. 66.

The exemplary main check valve 1800 illustrated in FIG. 63 is also provided with apparatus that prevents compression (or any substantial compression) of the valve seat 1830 and controls the compression of the gasket 1844. More specifically, the housing 1802 includes a stop member 1838 that extends from the end of the housing flow portion 1806. The exemplary stop member 1838 is a rigid, substantially annular structure that is integral with the housing flow portion 1806 and has a height H that is equal to, or is slightly less than (e.g. about 0.003 inch less than), the largest thickness of the valve seat 1830. The stop member 1838, which is located between the valve seat 1830 and the gasket 1844, limits movement of the main check valve 1800 relative to the structure into which the main check valve is inserted. More specifically, in the exemplary fluid transfer device 100r illustrated in FIG. 66, the stop member 1838 engages the end wall 119 of the valve recess 118. It should also be noted that the stop member 1838 may define a continuous perimeter (as shown) or, alternatively, be composed of a plurality of spaced elements that together define a discontinuous perimeter. The stop member 1838, whether continuous or discontinuous, may also have an overall shape other than the illustrated substantially annular shape.

Additionally, in the illustrated embodiment, the stop member 1838 includes a plurality of small co-molding holes 1839 (FIG. 65) that extend through the stop member in the radial direction (e.g. 4 holes with 90 degree spacing) that are used when the valve seat 1830 and gasket 1844 are simultaneously co-molded onto the housing 1802. Alternatively, the co-molding holes 1839 may be eliminated as is discussed below in the context of the co-molding process.

With respect to manufacturing and materials, the exemplary housing 1802 is a machined part and suitable materials for the housing include, but are not limited to, titanium, titanium alloys, stainless steel (e.g. 316L stainless steel), cobalt-nickel alloys, and refractory metals such as tantalum. The valve element 1804 may also be machined and suitable materials for the machined valve element include, but are not limited to, those described above in the context of the housing 1802. Alternatively, as discussed in greater detail below, the valve element 1804 may be molded and suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the valve seat 1830 and gasket 1844 include, but are not limited to, elastomers such as silicone rubber, latex rubber, urethane, butyl rubber, and isoprene.

The valve seat 1830 and gasket 1844 may be molded directly onto the housing 1802 by way of a co-molding (or “insert molding”) process. Here, the housing 1802 is clamped into a mold that includes cavities in the shape of the valve seat 1830 and gasket 1844, and the silicone rubber or other material is then injected into the cavities. A primer may be used to insure that the injected material sticks to the housing 1802. In those instances where the co-molding holes 1839 in the stop member 1838 are present, the material need only be injected into one of the cavities because it will flow through the co-molding holes to the other. Such co-molding is advantageous because it eliminates the tolerance stack-up that is associated with the assembly of separately manufactured parts. Nevertheless, the valve seat 1830 and gasket 1844 may, alternatively, be separately manufactured, positioned inwardly and outwardly of the stop member 1838 and, if desired, secured to the housing 1802 with adhesive.

There are a variety of advantages associated with the main check valve 1800. For example, compression of the valve seat 1830 in the exemplary main check valve 1800 is a function of only two dimensions, i.e. the thickness of the valve seat and the height H of the stop member 1838, as compared to the three dimensions associated with the main check valve 107 illustrated in FIGS. 1-5. It is, therefore, substantially less likely that a tolerance stack-up will result in over-compression of the valve seat 1830, as compared to the main check valve 107. Additionally, even if there is some over-compression of the valve seat 1830, the valve seat will not impinge the valve element shaft 1816 in those instance where the diameter of the opening 1833 is relatively large.

Turning to the gasket, the raised seal 1835 on the gasket 1844 in the exemplary main check valve 1800 will also be under compression when the main check valve is inserted into the valve recess 118 of the housing 102. Compressing the raised seal 1835 is advantageous because silicone and other elastomeric materials tend to tear when in tension. The raised seal 1835 also increases sealing pressure in a manner that is relatively easy to manufacture. Additionally, compression of the gasket 1844 is a function of only two dimensions, i.e. the thickness of the valve seat and the height H of the stop member 1838, as compared to the three dimensions associated with the main check valve 107 illustrated in FIGS. 1-5. It is, therefore, substantially less likely that a tolerance stack-up will result in over-compression of the gasket 1844, as compared to the main check valve 107. Additionally, even if there is some over-compression of the gasket 1844, the gap 1840 will allow the gasket to deform farther outwardly.

With respect to the relationship between the valve seat 1830 and the gasket 1844, the valve seat and gasket are separate structural elements and are spaced from one another. Compression (and over-compression) of the gasket 1844 will not, therefore, substantially effect the elastomeric valve seat 1830. Even in those instances where the valve seat 1830 and gasket 1844 are connected to one another by a small amount of elastomeric material that remains in the aforementioned co-molding holes in the stop member 1838, this connection will not result in compression (and over-compression) of the gasket substantially effecting the elastomeric valve seat.

Turning to the curved seal 1866, the curved seal reduces the contact area between the valve seat 1830 and the valve element head 1818, which in turn increases the sealing pressure, as compared to a sealing arrangement that has two flat surfaces. The curved seal 1866 also eliminates the adhesion force associated with flat on flat contact surfaces. The scuffing issue is also obviated because the valve element head 1818 does not have sharp lateral edges that come into contact with the valve seat 1830, which reduces stress risers.

It should also be noted here that, as alluded to above, the valve elements 304, 404, 504, 604, 704, 804, 904, 1004, 1104, 1204, 1304, 1404, 1504 ,1604 and 1804 (collectively valve elements “304-1604 and 1804”) may also be molded, as opposed to being machined. There are a variety of benefits associated with the molding of these valve elements. For example, molding tends to be less expensive than machining. Also, because the nature of machining processes can vary from lot to lot, there may be variations in the surface of the valve element heads and these variations may degrade the quality of the seal formed by the valve element and valve seat. A molded valve element is not subject to the same surface variation. The properties of the surfaces of the valve elements 304-1604 and 1804 may also be engineered through the use of particular materials. For example, coatings of non-stick coatings of materials such as silica, titania, PTFE, and silicone or silane based coatings from NuSil, may be used to help prevent the molded valve elements from sticking to the associated valve seats.

It should also be noted that various treatments may be employed in order to make surfaces within the valves 200-1600 and 1800 hydrophilic, thereby decreasing the likelihood that air bubbles will form on the surfaces and compromise the compression ratio. Such surfaces include, but are not limited to, the piston bore, the external surfaces of the piston, the valve elements, and the main check valve seat. With respect to titanium surfaces, a layer of hydrophilic titanium oxide may be formed by treating the titanium surface with a strong base (e.g. sodium hydroxide or potassium hydroxide) and/or a peroxide (e.g. hydrogen peroxide). With respect to elastomeric surfaces, such as the surfaces of valve seats, a hydrophilic surface may be formed by implanting titanium dioxide ions into the surface.

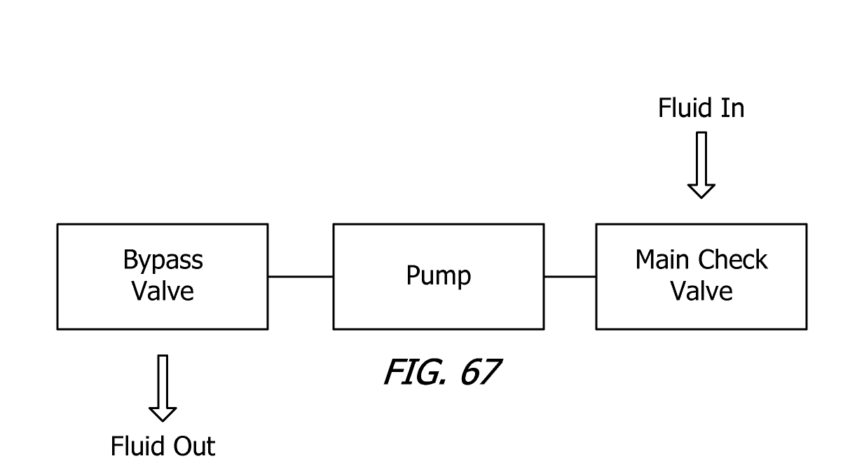

Referring to FIG. 67, and as discussed in detail above, fluid transfer devices in accordance with some of the present inventions may include a pump and a main check valve. The pump may be, by way of example but not limitation, an electromagnet pump, a solenoid pump, a piezoelectric pump, or any other mechanical or electromechanical pulsatile pump, the main check valve may be any of the main check valves 200-1800 described above and, accordingly, the present fluid transfer devices include any and all combinations of such pumps and main check valves. The present fluid transfer device may also include a bypass valve. One example of such a bypass valve is generally represented by reference numeral 106 in FIG. 1. Fluid entering such fluid transfer devices will typically pass the main check valve prior to being acted on by the pump, and will pass the bypass valve on its way to the outlet after being acted one by the pump.

The overall dimensions of the present main check valves will, of depend upon the particulars of the valve and the associated fluid transfer device. By way of example, an implementation of the main check valve 1500 which forms part of a fluid transfer device, such as that illustrated in FIGS. 1-5 that is itself sized for use in an implantable infusion device, has the following dimensions. The diameters of the housing fluid flow portion 1506 and the mounting portion 1510 are about 0.1 inch and about 0.3 inch, respectively. The length of the valve 1500, i.e. the combined lengths of the fluid flow portion 1506 and the mounting portion 1510 plus the thickness of the gasket 1544, is about 0.1 inch. The other valves will be similarly dimensioned in similar circumstances.

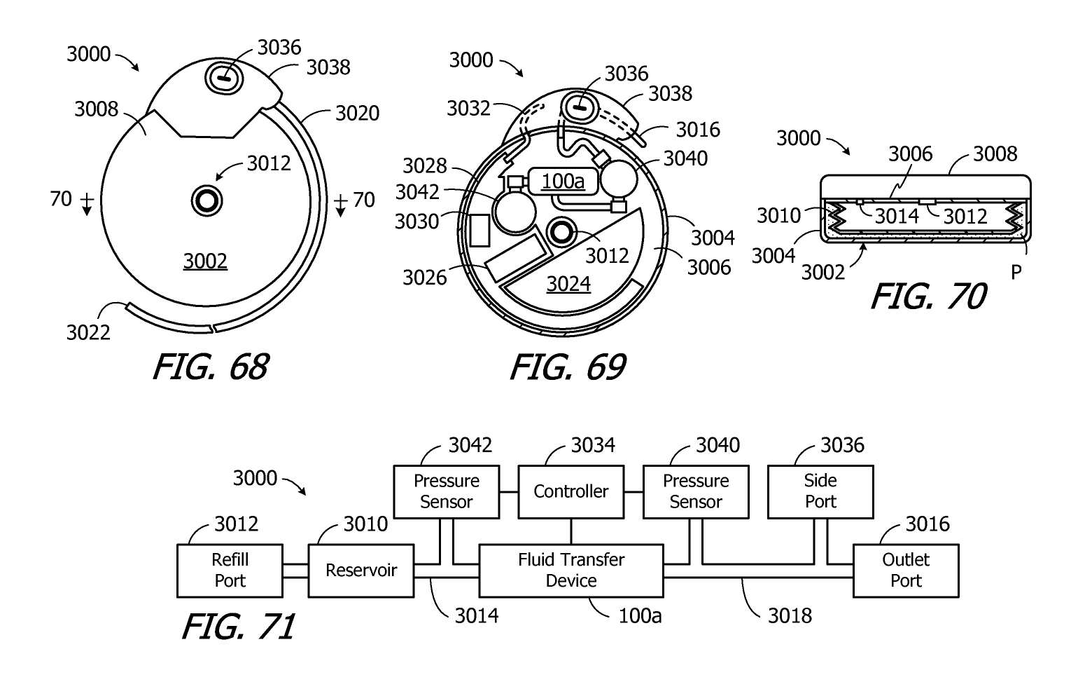

One example of an ambulatory infusion device in accordance with a present invention is the implantable infusion device generally represented by reference numeral 3000 in FIGS. 68-71. The exemplary infusion device 3000 includes a housing 3002 (e.g. a titanium housing) with a bottom portion 3004, an internal wall 3006, and a cover 3008. An infusible substance (e.g. medication) may be stored in a reservoir 3010 that is located within the housing bottom portion 3004. The reservoir 3010 may be replenished by way of a refill port 3012 that extends from the reservoir, through the internal wall 3006, to the cover 3008. A hypodermic needle (not shown), which is configured to be pushed through the refill port 3012, may be used to replenish the reservoir 3010.

The exemplary ambulatory infusion device 3000 also includes a fluid transfer device. Although the fluid transfer device 100a is shown, the ambulatory infusion device 3000 may, alternatively, include any of the fluid transfer devices 100b-100r described above. The inlet of a fluid transfer device 100a is coupled to the interior of the reservoir 3010 by a passageway 3014, while the outlet of the fluid transfer device is coupled to an outlet port 3016 by a passageway 3018. Operation of the fluid transfer device 100a causes infusible substance to move from the reservoir 3010 to the outlet port 3016. A catheter 3020 may be connected to the outlet port 3016 so that the infusible substance passing through the outlet port will be delivered to a target body region in spaced relation to the infusion device 3000 by way of the outlet 3022 at the end of the catheter.

A wide variety of reservoirs may be employed. In the illustrated embodiment, the reservoir 3010 is in the form of a titanium bellows that is positioned within a sealed volume defined by the housing bottom portion 3004 and internal wall 3006. The remainder of the sealed volume is occupied by propellant P, which may be used to exert negative pressure on the reservoir 3010. Other reservoirs that may employed in the present infusion devices include reservoirs in which propellant exerts a positive pressure. Still other exemplary reservoirs include negative pressure reservoirs that employ a movable wall that is exposed to ambient pressure and is configured to exert a force that produces an interior pressure which is always negative with respect to the ambient pressure.

Energy for the fluid transfer device 100a, as well for other aspects of the exemplary infusion device 3000, is provided by the battery 3024 illustrated in FIG. 65. In the specific case of the fluid transfer devices 100a-100r, the battery 3024 is used to charge one or more capacitors 3026, and is not directly connected to the fluid transfer device itself. The capacitor(s) 3026 are connected to the electromagnet coil in the fluid transfer device 100a, and disconnected from the battery 3024, when the electromagnet coil is being energized, and are disconnected from the electromagnet coil and connected to the battery when the capacitor(s) are being recharged and/or when the fluid transfer device is at rest. The capacitor(s) 3026 are carried on a board 3028. A communication device 3030, which is connected to an antenna 3032, is carried on the same side of the board 3028 as the capacitor(s) 3026. The exemplary communication device 3030 is an RF communication device. Other suitable communication devices include, but are not limited to, oscillating magnetic field communication devices, static magnetic field communication devices, optical communication devices, ultrasound communication devices and direct electrical communication devices.

A controller 3034 (FIG. 71), such as a microprocessor, microcontroller or other control circuitry, is carried on the other side of the board 3028. The controller controls the operations of the infusion device 3000 in accordance with instructions stored in memory (not shown) and/or provided by and external device by way of the communication device 3030. For example, the controller 3034 may be used to control the fluid transfer device 100a to supply fluid to the patient in accordance with, for example, a stored basal delivery schedule, or a bolus delivery request.

Referring to FIGS. 69 and 71, the exemplary infusion device 3000 is also provided with a side port 3036 that is connected to the passageway 3018 between the outlet of the fluid transfer device 100a and the outlet port 3016. The side port 3036 facilitates access to an implanted catheter 3020, typically by way of a hypodermic needle. For example, the side port 3036 allows clinicians to push fluid into the catheter 3020 and/or draw fluid from the catheter.

The outlet port 3016, a portion of the passageway 3018, the antenna 3032 and the side port 3036 are carried by a header assembly 3038. The header assembly 3038 is a molded, plastic structure that is secured to the housing 3002. The housing 3002 includes a small aperture through which portions of the passageway 3018 are connected to one another, and a small aperture through which the antenna 3032 is connected to the board 3028.

The exemplary infusion device 3000 illustrated in FIGS. 68-71 also includes a pressure sensor 3040 that is connected to the passageway 3018 between the outlet of the fluid transfer device 100a and the outlet port 3016. As such, the pressure sensor 3040 senses the pressure at the outlet port 3016 which, in the illustrated embodiment, is also the pressure within the catheter 3020. Another pressure sensor 3042 is connected to the passageway 3014 between the reservoir 3010 and the inlet of the fluid transfer device 100a. The pressure sensor 3042 senses the pressure at the inlet of the fluid transfer device 100a and may be used to measure the reservoir pressure. The pressure sensors 3040 and 3042, which are connected to the controller 3034, may also be used to measure the pressure differential across the fluid transfer device 100a.

The pressure measurements from the pressure sensor 3040 and/or the pressure sensor 3042 may be used to analyze a variety of aspects of the operation exemplary infusion device 3000. For example, pressure measurements may be used to determine whether or not there is a complete or partial blockage in the catheter 3020.

In the exemplary context of implantable drug delivery devices, and although the volume/stroke magnitude may be increased in certain situations, the fluid transfer devices will typically deliver about 1 milliliter/stroke or less.

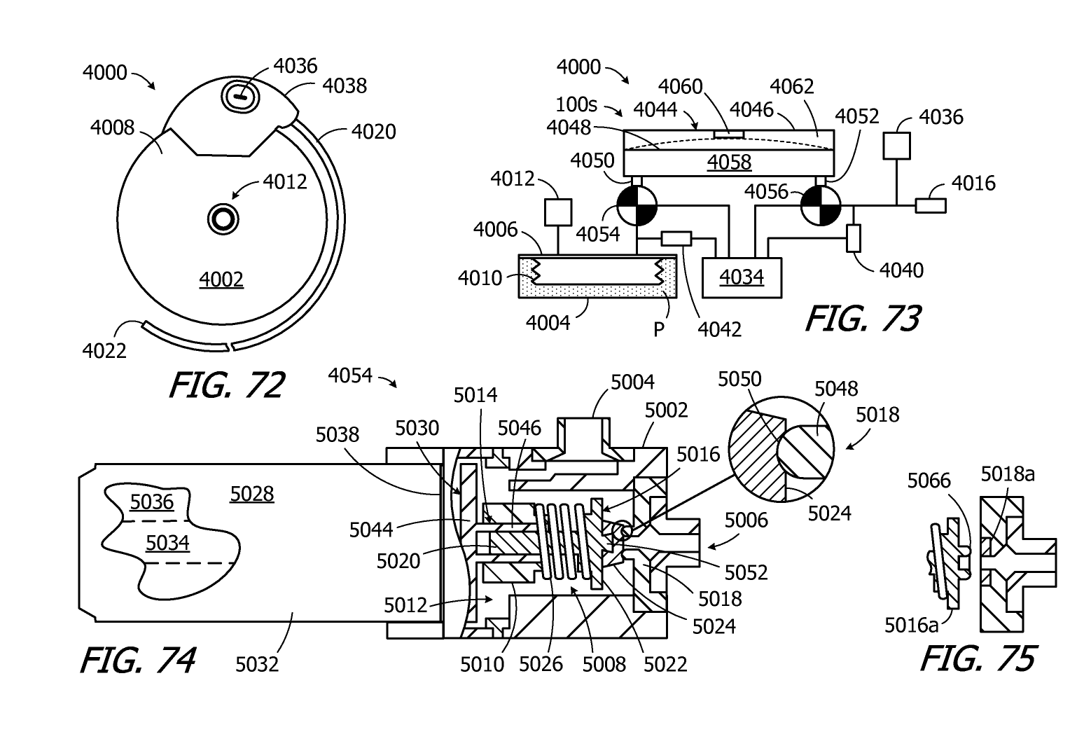

One example of an ambulatory infusion device in accordance with a present invention is the implantable infusion device generally represented by reference numeral 4000 in FIGS. 72 and 73. The implantable infusion device 4000 is similar to the implantable infusion device 3000 in many respects and similar elements are represented by similar reference numerals. To that end, the exemplary infusion device 4000 includes a housing 4002 (e.g. a titanium housing) with a bottom portion 4004, an internal wall 4006, and a cover 4008. An infusible substance (e.g. medication) may be stored in a reservoir 4010 that is located within the housing bottom portion 4004. The reservoir 4010 may be replenished by way of a refill port 4012 that extends from the reservoir, through the internal wall 4006, to the cover 4008. A hypodermic needle (not shown), which is configured to be pushed through the refill port 4012, may be used to replenish the reservoir 4010. The reservoir 4010 in the exemplary infusion device 4000 is a positive pressure reservoir and, in the illustrated embodiment, the reservoir is in the form of a titanium bellows that is positioned within a sealed volume defined by the housing bottom portion 4004 and internal wall 4006. The remainder of the sealed volume is occupied by a propellant P that exerts a positive pressure on the bellows.

The exemplary infusion device 4000 also includes a fluid transfer device 100s that is configured for use in combination with a positive pressure reservoir such as the exemplary positive pressure reservoir 4010. In the illustrated embodiment, the fluid transfer device 100s has an accumulator 4044 that includes a housing 4046, a diaphragm 4048 (e.g. a flexible sheet of titanium), an inlet 4050, and an outlet 4052. The fluid transfer device 100s also has an active inlet valve 4054, which controls the flow of fluid into the housing inlet 4050, and an active outlet valve 4056, which controls the flow of fluid out of the housing outlet 4052. The active inlet valve 4054 is also connected to the interior of the positive pressure reservoir 4010, while the active outlet valve 4056 is also connected to the outlet port 4016 which, in turn, may be connected to the catheter 4020. The exemplary active valves 4054 and 4056 are discussed in greater detail below with reference to FIGS. 74 and 75.

During operation of the fluid transfer device 100s, infusible substance will move from the positive pressure reservoir 4010 to an accumulator cavity 4058, which is defined by the housing 4046 and the diaphragm 4048, when the active inlet valve 4054 is opened. A pressure chamber 4062 is located on the other side of the diaphragm 4048. The active outlet valve 4056 will be closed while the inlet valve 4054 is opened. The diaphragm 4048 will flex due to the positive pressure from the reservoir until it reaches a stop 4060, as is shown in dashed lines in FIG. 73, thereby increasing the volume of the accumulator cavity 4058 by a predetermined amount. The active inlet valve 4054 will then be allowed to close. When the active outlet valve 4056 is opened, the pressure within the chamber 4062 will drive the diaphragm 4048 back to the solid line position, thereby driving the predetermined volume of fluid to the outlet port 4016.

Although the present fluid transfer device 100s is not so limited, the active inlet and outlet valves 4054 and 4056 in the illustrated embodiment are identical electromagnet valves that may be selectively actuated in a manner similar to the electromagnet pumps described above. Turning to FIG. 74, the exemplary active inlet valve 4054 (and outlet valve 4056) includes a generally solid, cylindrical housing 5002 with various open regions that accommodate portions of various structures and define a fluid flow path. More specifically, the housing 5002 includes an inlet 5004, an outlet 5006 and an open region 5008. The inlet 5004 may be used as an outlet, and the outlet 5006 may be used as an inlet, when the direction of fluid flow through the valve 4054 is reversed. A spring retainer 5010, with apertures 5012 to permit fluid flow and a bore 5014, is mounted within the housing 5002. A valve element 5016 is movable in to and out of engagement with a rigid valve seat 5018 that is associated with the outlet 5006. The valve element 5016 includes a shaft 5020, a spring retainer 5022, and an elastomeric seal 5024. A spring 5026 (e.g. a coil spring), which is mounted between the spring retainers 5010 and 5022, biases the valve element 5016 to the closed position illustrated in FIG. 74 such that the seal 5024 engages the valve seat 5018.

With respect to actuation, the exemplary active valve 4054 includes an electromagnet 5028 and an armature 5030. The electromagnet 5028, which is carried within a case 5032 that is secured to the housing 5002, has a core 5034 and a coil 5036. The case 5032 and core 5034 are made from a magnetic material. The coil 5036 consists of a wire or other conductor that is wound around the core 5034. The coil 5036 may be insulated from the case 5032 by electrically non-conductive spacers (not shown), which center the coil within the case, or through the use of potting compound or encapsulant material between the case and the coil. A barrier 5038 separates the open region 5008, which will ultimately be filled with fluid, from the electromagnet 5028. The armature 5030 consists of a pole 5044 formed from a magnetic material (e.g. magnetic steel), which is located within the open region 5008 such that it will be magnetically attracted to the electromagnet 5028 when the electromagnet is actuated, and a hollow cylindrically-shaped bushing 5046 that extends from the pole and into the bore 5014 and is slidable relative to the bore. The valve element shaft 5020 is fixedly secured (e.g. through a press fit) to the armature pole 5044 by way of the bushing 5046. The magnetic attraction between the actuated electromagnet 5028 and the armature pole 5044 is sufficient to overcome the biasing force of the spring 5026 and move the valve element 5016 away from the valve seat 5018 to open the active valve 4054.

The exemplary valve seat 5018 illustrated in FIG. 74, which is rigid and has an overall circular shape, includes a main portion 5048 and a curved surface 5050 that is semi-circular or otherwise curved in cross-section. The curved surface 5050 reduces the contact area between the valve seat 5018 and the valve element seal 5024, which in turn increases the sealing pressure, as compared to a sealing arrangement that has two flat surfaces. The curved surface 5050 also eliminates the adhesion force associated with flat on flat contact surfaces. The scuffing issue discussed above is also obviated because the rigid valve seat 5018 does not have sharp edges that come into contact with the elastomeric seal 5024.

In other implementations, a valve element 5016a (FIG. 75) may be provided with a curved seal 5066 that is semi-circular or otherwise curved in cross-section and protrudes from the spring retainer. The seal 5066 is rigid and engages an elastomeric valve seat 5018a when the valve is closed. This arrangement is analogous to, and provides the same benefits as, the curved seal arrangement associated with the exemplary valve 1300 illustrated in FIG. 41.

With respect to manufacturing and materials, the exemplary housing 5002 is a machined part and suitable materials for the housing include, but are not limited to, titanium, titanium alloys, stainless steel (e.g. 316L stainless steel), cobalt-nickel alloys, and refractory metals such as tantalum. The valve element 5016 (less the seal 5024) may also be machined and suitable materials for the machined valve element include, but are not limited to, those described above in the context of the housing 5002. Alternatively, the valve element 5016 (less the seal 5024) may be molded. Suitable materials for a molded valve element include, but are not limited to, polyolefins, liquid crystal polymers, PEEK, polyacetal plastics such as Delrin®, fluoropolymers, and most other molded materials that are rigid and inert to pharmaceuticals. Suitable materials for the elastomeric seal 5024 include, but are not limited to, elastomers such as silicone rubber, latex rubber, urethane, butyl rubber, and isoprene. The elastomeric seal 5024 may be molded directly onto the relatively rigid spring retainer 5022 by way of a co-molding process (note the anchor 5052). The elastomeric seal 5024 may, alternatively, be separately manufactured and secured to the relatively rigid spring retainer 5022 with adhesive.

Additional information concerning the exemplary fluid transfer device 100s and/or active valves may be found in U.S. Pat. Nos. 4,838,887 and 5,368,274, which are incorporated herein by reference. It should also be noted here that, although the active valves in the illustrated embodiments include electromagnet actuators, other types of actuators may also be employed. For example, solenoid and piezoelectric actuators may be employed.

Energy for the active valves 4054 and 4056, as well for other aspects of the exemplary infusion device 4000, is provided by the implantable infusion device battery (not shown). The battery charges one or more capacitors in the manner described above, and is not directly connected to the active valves themselves. The capacitor(s) are selectively connected to one of the electromagnet coils 5036 in the active valves 4054 and 4056, and disconnected from the battery, when an electromagnet coil is being energized, and are disconnected from the electromagnet coils and connected to the battery when the capacitor(s) are being recharged and/or when the fluid transfer device 100s is at rest.

As discussed above in the context of infusion device 3000, the capacitor(s) are carried on a board along with an RF communication device that is connected to an antenna. The communication device may, alternatively, be an oscillating magnetic field communication device, a static magnetic field communication device, an optical communication device, an ultrasound communication device, a direct electrical communication device, or other suitable device. A controller 4034 (FIG. 73), such as a microprocessor, microcontroller or other control circuitry, is carried on the other side of the board. The controller controls the operations of the infusion device 4000 in accordance with instructions stored in memory and/or provided by and external device by way of the aforementioned communication device. For example, the controller 4034 may be used to control the fluid transfer device 100s to supply fluid to the patient in accordance with, for example, a stored basal delivery schedule or a bolus delivery request, by selectively actuating (i.e. opening) and de-actuating (i.e. closing) the active valves 4054 and 4056.

Referring to FIGS. 72 and 73, the exemplary infusion device 4000 is also provided with a side port 4036 that is connected to a passageway between the outlet of the active valve 4056 and the outlet port 4016. The side port 4036 facilitates access to an implanted catheter 4020, typically by way of a hypodermic needle. For example, the side port 4036 allows clinicians to push fluid into the catheter 4020 and/or draw fluid from the catheter. The outlet port 4016, a portion of the associated passageway, the antenna and the side port 4036 are carried by a header assembly 4038. The header assembly 4038 is a molded, plastic structure that is secured to the housing 4002. The housing 4002 also includes a small aperture through which portions of the passageway are connected to one another, and a small aperture through which the antenna is connected to the board.

The exemplary infusion device 4000 may include a pressure sensor 4040 between the active valve 4056 and the outlet port 4016. As such, the pressure sensor 4040 senses the pressure at the outlet port 4016 which, in the illustrated embodiment, is also the pressure within the catheter 4020. Another pressure sensor 4042 may also be between the reservoir 4010 and the active valve 4054. The pressure sensor 4042 may be used to measure the reservoir pressure. The pressure sensors 4040 and 4042, which are connected to the controller 4034, may also be used to measure the pressure differential across the fluid transfer device 100s and to analyze a variety of aspects of the operation of the exemplary infusion device 4000. For example, pressure measurements may be used to determine whether or not there is a complete or partial blockage in the catheter 4020.

Although the inventions disclosed herein have been described in terms of the preferred embodiments above, numerous modifications and/or additions to the above-described preferred embodiments would be readily apparent to one skilled in the art. By way of example, but not limitation, the present inventions have application in infusion devices that include multiple reservoirs and/or outlets. Moreover, the inventions include any and all combinations of the elements from the various embodiments disclosed in the specification. It is intended that the scope of the present inventions extend to all such modifications and/or additions and that the scope of the present inventions is limited solely by the claims set forth below.

1. An ambulatory medical device, comprising:
a reservoir;
a pump; and
a valve in fluidic communication with the reservoir and the pump, the valve including
a housing including a fluid lumen,
an elastomeric valve seat associated with the housing and including an opening in fluid communication with the fluid lumen, and
a valve element including a shaft and a head carried by the shaft, the head having a main portion and a substantially rigid curved seal that protrudes from the main portion,
wherein the valve element is movable relative to the housing between a closed position where the curved seal engages the valve seat and an open position where the curved seal is in spaced relation to the valve seat.

2. An ambulatory medical device as claimed in claim 1, wherein the curved seal is substantially semi-circular in cross-section.

3. An ambulatory medical device as claimed in claim 2, wherein the curved seal defines a complex radius in cross-section.

4. An ambulatory medical device as claimed in claim 1, wherein the valve seat includes a flat sealing surface that is engaged by the curved seal when the valve element is in the closed position.

5. An ambulatory medical device as claimed in claim 1, wherein the valve element head main portion defines a perimeter and the curved seal extends around the perimeter of the main portion.

6. An ambulatory medical device as claimed in claim 1, wherein the valve element shaft extends through the housing fluid lumen and valve seat opening.

7. An ambulatory medical device as claimed in claim 1, further comprising:
a spring that biases the valve element to the closed position.

8. An ambulatory medical device as claimed in claim 1, wherein the reservoir comprises a bellows reservoir.

9. An ambulatory medical device as claimed in claim 1, further comprising:
a medical device housing configured for implantation into a human body;
wherein the reservoir, the pump and the valve are located within the medical device housing.

10. An ambulatory medical device as claimed in claim 1, wherein the pump comprises a pulsatile pump.

11-17. (canceled)

18. An ambulatory medical device, comprising:
a reservoir;
a fluid transfer device operably connected to the reservoir and including a fluid transfer device housing with a valve recess having an end wall and a pump associated with the fluid transfer device housing; and
a valve in fluidic communication with the pump, the valve including
a valve housing including a fluid lumen,
an elastomeric valve seat associated with the valve housing and including an opening in fluid communication with the fluid lumen,
a stop member associated with the valve housing, and
a valve element associated with the fluid lumen and movable relative to the housing between a closed position where the valve element engages the elastomeric valve seat and an open position where the valve element is in spaced relation to the elastomeric valve seat.

19. An ambulatory medical device as claimed in claim 18, wherein the stop member is integral with the valve housing.

20. An ambulatory medical device as claimed in claim 18, wherein the stop member is located outwardly of the elastomeric valve seat.

21. An ambulatory medical device as claimed in claim 18, wherein the stop member comprises a substantially annular structure.

22. An ambulatory medical device as claimed in claim 18, wherein
the elastomeric valve seat defines a thickness; and
the stop member defines a height that is substantially equal to the thickness of the elastomeric valve seat.

23. An ambulatory medical device as claimed in claim 18, wherein
the housing includes a fluid flow portion through which the fluid lumen extends, the fluid flow portion defining an outer perimeter; and
the stop member is located inwardly of the outer perimeter of the housing fluid flow portion and outwardly of the elastomeric valve seat.

24. An ambulatory medical device as claimed in claim 13, further comprising:
a gasket located outwardly of the stop member.

25. An ambulatory medical device as claimed in claim 24, wherein the gasket defines an outer perimeter; and
the outer perimeter of the gasket is located inwardly of the outer perimeter of the housing fluid flow portion.

26. An ambulatory medical device as claimed in claim 24, wherein the stop member defines a height; and
the gasket defines a thickness that is substantially greater than the stop member height.

27. An ambulatory medical device as claimed in claim 24, wherein the stop member includes a plurality of co-molding holes.

28-33. (canceled)

34. An ambulatory medical device, comprising:
a reservoir;
a pump; and
a valve in fluidic communication with the reservoir and the pump, the valve including
a valve housing including a fluid lumen,
an elastomeric valve seat associated with the valve housing and including an opening in fluid communication with the fluid lumen,
a gasket associated with the valve housing,
the elastomeric valve seat and gasket being respectively constructed and arranged such that the compression of the gasket will not substantially effect the elastomeric valve seat, and
a valve element associated with the fluid lumen and movable relative to the valve housing between a closed position where the valve element engages the elastomeric valve seat and an open position where the valve element is in spaced relation to the elastomeric valve seat.

35. An ambulatory medical device as claimed in claim 34, further comprising:
a stop member associated with the housing and positioned between the gasket and the elastomeric valve seat.

36. An ambulatory medical device as claimed in claim 35, wherein the stop member includes a plurality of co-molding holes.

37. An ambulatory medical device as claimed in claim 34, wherein
the housing includes a fluid flow portion through which the fluid lumen extends, the fluid flow portion defining an outer perimeter; and
the gasket defines an outer perimeter that is located inwardly of the outer perimeter of the housing fluid flow portion.

38-39. (canceled)

40. An ambulatory medical device as claimed in claim 34, wherein the gasket is located in spaced relation to the elastomeric valve seat.

41. An ambulatory medical device as claimed in claim 34, wherein the gasket includes a base and a raised seal that extends from the base.

42-45. (canceled)
